(19) United States
(12) Patent Application Publication
(10) Pub. No.: US 2026/0081034 A1
(43) Pub. Date: Mar. 19, 2026

(54) SYSTEMS AND METHODS FOR MEASURING, LEARNING, AND USING EMERGENT PROPERTIES OF COMPLEX ADAPTIVE SYSTEMS

(71) Applicant: EMERJA CORPORATION, Monte Sereno, CA (US)

(72) Inventors: Guy Miller, Monte Sereno, CA (US); Daniel Hansen, Santa Cruz, CA (US); Arthur E. Colvin, Mt. Airy, MD (US)

(21) Appl. No.: 19/399,270

(22) Filed: Nov. 24, 2025

Related U.S. Application Data
(63) Continuation of application No. 18/042,997.
(60) Provisional application No. 63/071,989, filed on Aug. 28, 2020.
(60) Provisional application No. 63/071,982, filed on Aug. 28, 2020.
(60) Provisional application No. 63/090,610, filed on Oct. 12, 2020.
(60) Provisional application No. 63/181,913, filed on Apr. 29, 2021.

(51) Int. Cl. G16H 50/30; G16H 20/00; G16H 40/67; G16H 50/20; G16H 50/80

(52) U.S. Cl. CPC G16H 50/30; G16H 20/00; G16H 40/67; G16H 50/20; G16H 50/80

(57) ABSTRACT

Systems are described for measuring, recording, transmitting, accessing, and using an array of physical and physiological measurements that quantify states of complex adaptive systems, such as biological systems, more particularly the state is reflected in a metric designated health capacity. These measurements may be used, for example, for the pre-symptomatic detection and interception of disease states in a biological system. In one aspect, the system comprises a wearable device configured to measure, substantially simultaneously, an array of water-associated metrics, preferably at multiple loci on the biological system and as a function of time. The systems may further comprise using a scalable technology platform to identify, from the array of data across multiple systems, preferably compared to a training data set, utilizing machine readable instructions, to determine and/or predict health states, including the health capacity, of the biological system and to generate recommendations, including modification or nutrition, sleep, physical or mental inputs for the improvement of health for the biological system.

### CROSS-REFERENCE TO RELATED APPLICATIONS

This application is a continuation of U.S. patent application Ser. No. 18/042,997 filed Feb. 24, 2023, entitled “SYSTEMS AND METHODS FOR MEASURING, LEARNING, AND USING EMERGENT PROPERTIES OF COMPLEX ADAPTIVE SYSTEMS,” which is the national phase under 35 U.S.C. § 371 of International Patent Application No. PCT/US2021/048053 filed Aug. 27, 2021, entitled “SYSTEMS AND METHODS FOR MEASURING, LEARNING, AND USING EMERGENT PROPERTIES OF COMPLEX ADAPTIVE SYSTEMS,” which claims the benefit of priority under 35 U.S.C. § 119 (e) to U.S. Provisional Patent Application No. 63/181,913 filed Apr. 29, 2021, entitled “SYSTEMS AND METHODS FOR MEASURING, LEARNING, AND USING EMERGENT PROPERTIES OF COMPLEX ADAPTIVE SYSTEMS,” U.S. Provisional Patent Application No. 63/090,610 filed Oct. 12, 2020, entitled “SYSTEMS AND METHODS FOR MEASURING, LEARNING, AND USING EMERGENT PROPERTIES OF COMPLEX ADAPTIVE SYSTEMS,” U.S. Provisional Patent Application No. 63/071,989 filed Aug. 28, 2020, entitled “SYSTEMS AND METHODS FOR MEASURING, LEARNING, AND USING EMERGENT PROPERTIES OF COMPLEX ADAPTIVE SYSTEMS,” and U.S. Provisional Patent Application No. 63/071,982 filed Aug. 28, 2020, entitled “SYSTEMS AND METHODS FOR MEASURING, LEARNING, AND USING EMERGENT PROPERTIES OF COMPLEX ADAPTIVE SYSTEMS.” The disclosure of each of these prior applications is hereby incorporated by reference herein in its entireties.

### BACKGROUND

### Field of the Disclosed Technology

The disclosed technology generally relates to systems for, devices for, and methods of obtaining measurements of emergent properties of a complex adaptive system, such as a biological system, an organism, or for example a human, or a non-biological system, such as for example a protocell. The disclosed technology also generally relates to using such measurements to quantify states or properties of a biological or non-biological system, such as identifying or diagnosing, including pre-symptomatic diagnosis, one or more disease states, for example pathogen (for example, viral, bacterial) infections, of a biological system, such as an organism, for example a human, or for, for example measuring, assessing and controlling a protocell used for industrial manufacturing. The disclosed devices, systems, and methods generally allow for measurement, assessment, learning, and uses of states or properties of the biological system or non-biological system, including a property designated “health capacity,” and acting on such states or properties, including intercepting, treating or otherwise addressing disease states. The disclosed technology also generally relates to a wearable, implanted, embedded, or otherwise-coupled devices for obtaining measurements, and to a scalable technology platform to manipulate the data, including such functions as to compile, store, distribute, analyze, and use the measurements from one or more devices to predict states or properties of a biological system, such as an organism, for example a human.

### Description of the Related Art

The current approach to solving problems in the biological sciences is a bottoms-up approach often referred to as “precision biology.” In its most generalized form precision biology couples machine learning with a detailed measurement (“-omics”) of biological parts to ascribe function to parts. This approach is also often referred to as “precision medicine,” especially when applied to the discovery, development and delivery of healthcare solutions.

Precision biology is based on the concept that knowledge gaps are due to a lack of understanding of “parts,” and that detailed measurement and analysis will fill in these knowledge gaps. For example, a basic tool of modern biological sciences is organic chemistry, the study of carbon-containing molecules, and carbon bonded to other key atoms. This is, at least in part, because the genetic, signaling, and structural molecules of living systems consist largely of carbon atoms. Under the precision biology paradigm, function and prediction of function is therefore sought through quantifying these organic chemical attributes in greater detail. However, the art generally fails to recognize that many characteristics of biological systems do not lend themselves to the precision biology paradigm. One such example, where the precision biology paradigm fails, is in the measurement and prediction of emergent properties of complex adaptative (biological) systems. Emergent properties are properties of a system not found in a part, or readily deducible from a detailed inventory and analysis of the parts contained within a system. The precision biology approach—which today has risen to the level of a paradigm—has a blind spot with respect to emergent properties; this blind spot substantially limits advances in the fields of biology and medicine.

An historic perspective sheds light on the manifold advantages of the current technology, even as all past and current understandings of biological systems, and of how to monitor them and maintain or improve health neither anticipate nor render obvious the systems, methods, and uses of the current technology.

The measurement of human biological systems to understand their function can be traced as far back as Hippocrates in ˜450 BC. Hippocrates is credited for separating medicine from religion in human biological systems, and thus establishing a physical basis for measuring and diagnosing disease and developing prognosticators. The notion that human biology could be understood through the prism of physical sciences and not religion was advanced over the ensuing ˜2,400 years to the beginning of the industrial revolution.

Informed by advances in the industry, biologists and chemists in the late 19th century and early 20th century began to adopt analogous approaches and technologies to those successfully employed in physics, engineering, and industry. Over the first half of the 20th century, these approaches were parlayed into the successful identification of food-derived enzyme cofactors-vitamins; the biological basis of viral and bacterial infections and the means to intercept them-vaccines and antibiotics; and the successful identification of genetic material-DNA, and how genetic information encodes for proteins. These advances contributed to astonishing gains in the understanding of the biological sciences and medicine.

In the most general sense, the tools and reasoning that drove industrialization were then, and are now still, employed to solve biological problems. At the core of the reasoning is a form of reductionism—that is instantiated in the scientific method in the generalized hypothesis of “what part is ascribed to what function.”

A precision approach to learning would be most predictive when there is a simple and orderly relationship between a part and its function. Such examples in non-biological systems might include a tire on a bicycle, or in a biological system, a gene and protein critical for energy synthesis and life itself. In these instances, the measurement of the tire or the gene would be expected to correlate with the function of the system. The precision biology approach has its greatest positive predictive value in non-biological systems that are designed and engineered by humans, as these systems, by definition, follow a 1:1 relationship between parts and function. The precision biology approach also has value in biological systems where there is a hypothesized and measurable relationship between a part and its function. However, the precision biology approach would lose its positive predictive value in biological systems when there is not a 1:1 relationship between a part and its function. The instance where there is no discernable relationship between a part and its function is the case where a part, or two or more parts, form a new structure or perform a new function not readily predictable or discernable from the part or parts alone. This property, the ability of one or more parts to form a structure or perform a function not resident in the/a part alone, is its “emergent structure or function” and collectively its “emergent property or properties.”

The current precision biology approach to understanding the function of biological and complex non-biological systems is limited by the inability to measure, quantify, predict, control, maximize, design, and engineer complex adaptive biological and non-biological systems based on their emergent properties. These limitations are observed at nearly all hierarchies of biological systems, including the biosphere itself.

The precision biology approach is limited in understanding biological and human function. The parts-based approach implicitly considers the biological system or human as a self-contained collection of parts from which function is to be sorted and calculated. It embodies a pre-vitamin paradigm and ironically is limited today by an incomplete inventory of the parts responsible for function. Until relatively recently, the precision biology approach in humans omitted ˜1-10 trillion bacteria that form the human microbiome. The precision biology approach also discounts or omits in their entirety other parts and the context in which they exist. Examples would include food. While there are estimated to be over 30,000 plant-derived small molecules called phytonutrients in the human diet, the function of only <0.1% of these phytonutrients—the vitamins—are understood. Additionally, the precision biology approach discounts the criticality and importance of certain classes of enzymes and deprioritizes their study. This would include but is not limited to those in metabolism that interact with substances derived from the milieu exterior, such as oxidoreductase enzymes. When applied to medicine, the precision biology paradigm oversimplifies the complexity of biological systems. It is very limited in its ability to diagnose and develop treatments to disease when the function it seeks to understand is emergent in origin.

The precision biology approach is also limited in understanding the health of a species or collection of species and resources. The precision biology approach considers health as the null case or absence of disease obtained through a process of disease elimination. Today, human health is a concept, not an actuality. It should be noted that this was/is not always the case in many non-reductionist cultures. The concept of health as an energy state—essentially an emergent property—independent from disease is common to many eastern cultures and religions, chakras, reiki, prana, chi, can be traced to 400 BC, and more recently in the West as élan vital in the 1900s. Health is essentially the baseline of function of a biological system, and may also be referred to as homeostasis. But homeostasis is an emergent property: a complex interplay of many parts to produce interchangeable forms and functions that are not resident in or discernable by the precision biology approach. The precision biology approach significantly fails in an open system such as homeostasis (health) that is dependent upon the complex interaction of the milieu interior and milieu exterior. As a result, disease measures are used to define the absence of health. Disease measures are very poor surrogates for absence of health in that they substantially lag changes in health or “health capacity”: the resilience (adaptivity) of a system expressed primarily by its ability to persist or achieve some core function.

The precision biology approach is also limited in “genetic engineering” and industrial biology. The absence of a 1:1 relationship between the change of a gene and the intended outcome frequently results in the generation of non-homeostatic (un-healthy) states inconsistent with the intended new (synthetic function) or viability.

The precision biology approach is also limited in terms of its implementation. It requires highly specialized and expensive equipment for measurement. The precision biology approach is limited in terms of learning rate. It results in an unmanageable number of false (positive) discoveries and discounts the possibility of unexpected outcomes-“unintended consequences”. The cost, risk and time to learn are very high an increasing-Eroom's Law. The precision biology approach is limited by its invasiveness and ethicality. Whereas emergent properties are often quantifiable from the exterior, the precision biology approach typically involves invasive tests or experimental euthanasia that can be unethical, injurious or lethal-all of which reduce the practicality of gaining frequent and sufficient measurements. Without large sample sizes and real human test data, it is extremely difficult to obtain the necessary statistical power to differentiate good hypotheses from bad. This exacerbates the problems of false discovery and further slows the learning rate of the biological sciences generally. The precision biology approach is limited by its silence on anthropocentric effects on biological function. It considers more or less that DNA contains all of the relevant biological information, and that function cascades forthwith. It does not consider other physical or biological information systems-temperature, inter-/intra-species dependencies, gravity, magnetic fields, currents, populations-all of which have undergone dramatic changes in the last 100 years of the “anthropocene.” The precision biology approach is also limited by the conjecturing of a “paradigm”—“DNA is the book of life”—as being “truth” despite evidence to the contrary. Despite these clear limitations to the precision biology approach/paradigm, there is dogmatic continuation of this approach as a series of “-omics” revolutions. Because there may be an infinite set of taxonomies of parts, the parts-based approach is essentially inexhaustible and therefore, non-falsifiable: there is no means by which the precision biology paradigm can prove itself wrong. This non-falsifiability has been made worse by modern machine learning tools. It is now conjectured that the knowledge gap in the precision biology approach is not the approach, but insufficiencies in analytical methods to understand the parts. While machine learning tools will certainly have utility for those problems solvable by the precision biology approach, more analysis and data gathering has never supplanted the need for new measurements that make visible what has been hidden. The precision biology approach simply does not measure or acknowledge emergent properties of biological systems by which the essence of life is defined.

What is needed, therefore, is a new way to measure and quantify emergent properties of biological and non-biological systems to understand the function of complex adaptive systems enabling prediction, optimization, design, and engineering of biological and non-biological systems. This way should be considered as part of a “consilience approach” that considers the totality of all biological and non-biological parts and systems and their emergent properties within.

The SARS-COV-2 pandemic of 2019 highlights deficiencies of the precision biology approach when applied to medicine, and the need for a new approach. The current precision medicine approach seeks to understand precisely the disease state of the world—are individuals infected, and whether they have been conferred immunity. This is both impractical, slow and expensive. What is required is an efficient way to measure, not disease, but changes in health and, thus, intercept asymptomatic spread of the pathogen and containment. Because the baseline of health-homeostasis—is an emergent property, the measurement of health has been overlooked and unobtainable by the current precision biology/precision medicine approach.

The American Heart Association's has stated that Cardiorespiratory Fitness (CRF) is an important marker of health, and that CRF may be a clinically significant, wholistic metric for assessing health. See, e.g., Ross, Robert, et al. “Importance of assessing cardiorespiratory fitness in clinical practice: a case for fitness as a clinical vital sign: a scientific statement from the American Heart Association.” Circulation 134.24 (2016): e653-e699; Raghuveer, Geetha, et al. “Cardiorespiratory fitness in youth: an important marker of health: a scientific statement from the American Heart Association.” Circulation 142.7 (2020): e101-el18. CRF refers to the capacity of the circulatory and respiratory systems to supply oxygen to skeletal muscle mitochondria for energy production needed during physical activity. CRF may be estimated as the maximal oxygen uptake, which is measured via open-circuit spirometry during a maximal exercise test, for example. CRF is, however, in several ways, an undesirable metric. For example, apparatuses for measuring CRF are not wearable, CRF measurement and analysis suffer from latency, and CRF monitoring is not continuous. Furthermore, the cost of assessing CRF may be high, CRF is an indirect (and not necessarily accurate) measurement of metabolic health, and the CRF measuring procedures may require skilled personnel and healthcare professionals. Accordingly, CRF measurement may be inaccurate and non-scalable. As a result, CRF may be inadequate to be used as a viable and easily accessible health indicator.

New systems and methods are needed to understand, with greater accuracy, accessibility, scalability and more readily, the function of complex adaptive systems; such system would, ideally, enable prediction, optimization, design, and engineering of biological and even non-biological systems, and would consider the totality of all biological and non-biological parts and systems.

### SUMMARY

A novel approach to measurement and characterization of biological systems that capture the “emergent properties” of biological systems using measurements of attributes of the “emergent whole,” rather than per-se sum of parts, is disclosed. The novel approach relates to measurement of “health capacity,” which refers the resilience (adaptivity) of a system expressed primarily by its ability to persist or achieve some core function.

The novel generation, analysis, and use of data relating to the health capacity of biological systems is also disclosed. Novel sensors, and combinations of sensors, for capturing data related to the health capacity of biological systems is further disclosed.

The role of organic or carbon chemistry has been explored in assessing biological systems, particularly their selective chemistry, but the role of hydrogen, oxygen, and hydrogen bonding in biological systems has not. Hydrogen, oxygen, and hydrogen bonds are abundant in biological systems accounting for the vast majority of biological energy and entropy and therefore adaptation, yet the role and influence of the properties, functions, variability, response to stimuli, and other activities of hydrogen, oxygen, hydrogen-oxygen covalent bonds, oxygen-oxygen covalent bonds, hydrogen bonding, and other interactions involving hydrogen and/or oxygen on the functioning biological systems have not been adequately addressed.

Properties of water and/or aqueous systems, as exemplified and described herein, may be utilized to undergird approaches in the monitoring, assessment, and control of biological systems generally, including the monitoring, assessment, research, maintenance, and control of health, metabolism, homeostasis, stress, pre-inflammation, inflammation, various organ specific properties, various systemic properties, infection, and/or disease states, in improving understanding of the function and properties of biological systems. The disclosed technology is based, at least in part, upon and involves useful application of the insight that, water and aqueous systems, particularly as a component of a biological system, exhibit various properties, including those detailed herein. Such properties may be related, either directly or indirectly, to one or more states of the biological system.

In some aspects of the disclosed technology measurement devices, or an array of measurement devices, and a learning engine are provided. These devices and arrays of devices may be designed and/or adapted to generate data that, in turn, may be used to quantify the health capacity of a biological system. In certain preferred embodiments, the health capacity is based on and/or a function of one or more of the inherent or emergent properties of water and may be used to predict the functions or disease states of a biological system and/or to optimize, design and engineer biological or synthetic biological systems. One example of an advantage of such a measurement device or array of measurement devices and learning engine is that water is present in living systems and structures the modes by which living systems adapt to environmental stress(es). Water contributes to the ability of living systems to adapt because water possesses certain physical and chemical properties, including the ability to self-organize, dissipate heat, and establish fast, complex communication networks.

In some embodiments, the measurement device measures the physical and chemical properties of water, directly or indirectly, to quantify health capacity of a biological system. In some embodiments, the measurement device measures the thermodynamic, electrochemical and structural properties of water. In some embodiments, the measurement device measures the emergent properties of water with a small number of ions, molecules and elements. In some embodiments, the measurements are non-invasive. In some embodiments, the measurements are continuous. In some embodiments, the learning engine can quantify the baseline function, or the homeostasis or physiologic reserve of a biological system. In some embodiments, the learning engine can detect changes in the baseline function. In some embodiments, the learning engine can maximize the function. In some embodiments, the learning engine can design and engineer biological and non-biological chemical systems. In some embodiments, the learning engine can detect negative inflections or frailty in health capacity before standard clinical indices of disease can be detected. In some embodiments, the learning engine can detect positive inflections of health capacity, or fitness, when health interventions are applied, such as changes in food, exercise, sleep, or lifestyle. In some embodiments, the learning engine can generate information which is useful in the design and engineering of biological systems.

Systems are provided for quantifying a health capacity of a biological system, and/or learning the “health capacity rules” comprising: at least one sensor configured to measure an emergent factors of the biological system and generate measured data based on the emergent factor; and a processing system comprising a processor and an interface for receiving the measured data from the at least one sensor and determining one or more factors that quantify the health capacity of the biological system based on the measured data. The processor may be configured to compute, preferably according to one or more machine readable instructions, a solution for maximizing the health capacity of the biological system. The biological system may be an organism, such as a person. The system may further comprise a storage component in communication with the processing system and for storing the measured data. The measured data may be a health metric of the biological system. The processing system may comprise a plurality of transmitters configured to transmit the measured data as data streams optimized with respect to properties of the at least one sensor and the emergent factors to be reported. The processor may be configured to detect a disease state when it is pre-symptomatic. The processor is configured to make pre-symptomatic detection, preferably according to one or more machine readable instructions, of the disease state of the biological system using a supervised learning algorithm with a collection of health metrics reported from a plurality of other objects. The disease state is selected from, for example, aging, sepsis, cardiovascular disease, diabetes, malnutrition, cancer, lung disease, stroke, Alzheimer's, kidney disease, and infectious disease, and the infectious disease may be caused by a bacterial infection or a viral infection, such as, for example, a respiratory infection, a gastrointestinal tract infection, a liver infection, a nervous system infection, and a skin infection, or a coronavirus, such as SARS-CoV-2, which causes the disease condition known as Coronavirus Disease 2019 or COVID-19. At least one sensor may be a temperature or heat flux sensor, a barometric pressure sensor, a relative humidity sensor, a light sensor and others that quantify biological work such as redox sensor, an electrochemical sensor, a structural sensor, a tensile sensor, a motion sensor, or a combination thereof. The sensor may be a plurality of wearable devices, or an implanted device. The interface may transmit the measured data via wireless communication. The system, in certain embodiments, generates an output that includes a solution for intercepting a disease state, and may include the implementation of that solution. The system may further comprise an application program interface controller that controls storage of the measured data, access to the measured data, security configurations, user inputs, and output of any results. For example, FIG. 17 illustrates an embodiment showing how an application program interface can be used to build a “digital health marketplace.” At least one of the measuring devices measures a thermal property, a work property, or an environmental property of the biological system or which the biological system exists.

Certain systems may be configured to generate, based on an input training set, measured data comprising heat flux data. In certain systems, at least one health capacity may be a basal metabolic status, and at least one emergent factor is the temporal alignment of heat production and heat elimination of the biological system. In certain systems, the temporal alignment is related to at least one quasiperiodic rhythm of the biological system, and the quasiperiodic rhythm is a circadian rhythm. In certain systems, the processing system may be configured to automatically generate and output at least one indicator for improving or modulating the temporal alignment of heat production and heat elimination of the biological system, and the indicator may suggest performing to at least one predefined action selected from the group of actions comprises: change clothes, go inside, go outside, eat a specific food, drink a specified beverage, perform certain exercises, go to sleep, or any combination thereof, and the at least one indicator may suggest automatically administering suitable amounts of one or more of the following: a decoupling agent (sometimes referred to as an “uncoupler” or an “uncoupling agent”), a modulator of the oxidative phosphorylation pathway, a modulator of transmembrane ionic gradients, or any combination, and the indicators are used to manage circadian rhythm. Decoupling agents are molecules that disrupts oxidative phosphorylation on prokaryotes and mitochondria, or disrupts photophosphorylation in chloroplasts and cyanobacteria. Such molecules are capable of transporting photons through mitochondrial and liid membrances.

Methods are also provided for quantifying a health capacity of a biological system, comprising: sensing at least one emergent factors of the biological system, generating measured data relating to the at least one emergent factor, and determining, based on the measured data, one or more stimuli that influence the health capacity of the biological system. These methods may also comprise, preferably according to one or more machine readable instructions, generating a solution for maximizing the health capacity by modifying one or more stimuli that influence the health capacity of the biological system and implementing the solution by modifying the stimuli to increase the health capacity of the biological system. The stimuli is selected from, for example, sleep patterns, sleep duration, nutritional intake, an exercise regimen, or the presence of disease, for example, an infection, such as a viral infection, for example, infection by SARS-COV-2, which causes the disease condition known as Coronavirus Disease 2019 or COVID-19.

In certain methods, at least one health capacity is a basal metabolic status, and at least one emergent factor is the temporal alignment of heat production and heat elimination. The temporal alignment may be at least one quasiperiodic rhythm of the biological system, such as a circadian rhythm. Certain methods further comprise the step of generating and outputting at least one indicator for improving or modulating the temporal alignment of heat production and heat elimination of the biological system. The indicator may suggest performing to at least one predefined action selected from the group of actions comprises: change clothes, go inside, go outside, eat a specific food, drink a specified beverage, perform certain exercises, or any combination thereof, or recommending at least one predetermined action to improve or modulate the temporal alignment of heat production and heat elimination of the biological system, and the action may manage circadian rhythm.

In other embodiments, the system determines an energy signature of a non-biological system, and comprises at least one sensor configured to measure an emergent factor of the system and generate measured data based on the emergent factor; and a processing system comprising a processor and an interface for receiving the measured data from the at least one sensor and determining one or more factors that quantify an energy budget of the non-biological system based on the measured data. The non-biological system may be, for example, a model of a biological system, which can be configured to test interventions and engineer modifications to the real world counterpart of the modeled biological system. The non-biological system may be, for example, a synthetic system or an industrial system, and such synthetic systems and industrial systems may be configured to test interventions and engineer modifications to the synthetic system or industrial system. The non-biological system may also be an encryption system, and ecological system, and insurance pricing system, a cybernetic system, a gaming system, and a biomimetic system, each of which may be configured to use at least one energy signature to optimize the operation of the non-biological system. The system, in certain embodiments, generates an output that includes a solution for intercepting a disease state, and may include the implementation of that solution. In certain embodiments, the system is configured to use at least one energy signature to optimize the operation of the non-biological system, and may also be configured to implement such an optimization.

### BRIEF DESCRIPTION OF THE DRAWINGS

FIG. 1 illustrates, graphically, the concept of health capacity.

FIG. 2 illustrates, graphically, that symptoms are late indicators of loss of health.

FIG. 3 illustrates, graphically, that measures of energy are early indicators of loss of health.

FIG. 4 illustrates, schematically, an embodiment describing a learning strategy using measurement of energy and annotations to learn rules of health capacity.

FIG. 5 illustrates, in a Sankey diagram, an embodiment of a model of energy budget.

FIG. 6 shows an example of an embodiment of a device for measuring energy.

FIG. 7 illustrates, as a schematic, an example of the electronics of a device for measuring energy.

FIG. 8 shows, in cross-sectional views, a depiction of a device for measuring energy.

FIG. 9 shows, in a top view, a depiction of a holder for a device for measuring energy.

FIG. 10 illustrates, schematically, an example of how a device for measuring energy can be oriented, or sit, within a system that stores, processes, communicates, analyzes, and displays data and information.

FIG. 11 illustrates, as a graphical plot of Δ T v. time, an example of an embodiment of an output, in this instance from a device that measures energy, “energy signature,” as measured on a human over 30 days.

FIG. 12 illustrates, as a graphical plot of Δ T v. time, an example of an embodiment of an output, in this instance from a device that measures energy, “energy signature,” as measured in the (lower) exploded view on a human over 1 days, and illustrates, in the (upper) condensed view, such an exemplar embodiment of this output measured on a human for 30 day.

FIG. 13 illustrates, as graphic plot of energy expenditure v. time, an annotation of “energy signatures” with “metabolic tasks” to “quantify metabolism.”

FIG. 14 illustrates, schematically, an example of how a learning strategy yields embodiments based on insights.

FIG. 15A and FIG. 15B illustrate, schematically, graphically, and pictorially, certain embodiments in which learning and value are generated. FIG. 15A, in particular, illustrates learning and value generation in human health; FIG. 15B, in particular, illustrates learning and value generation in human disease.

FIG. 16 illustrates, schematically, a summary of learning methods and the generalized application of learning methods.

FIG. 17 illustrates, schematically, an embodiment showing how an application program interface can be used to build a “digital health marketplace.”

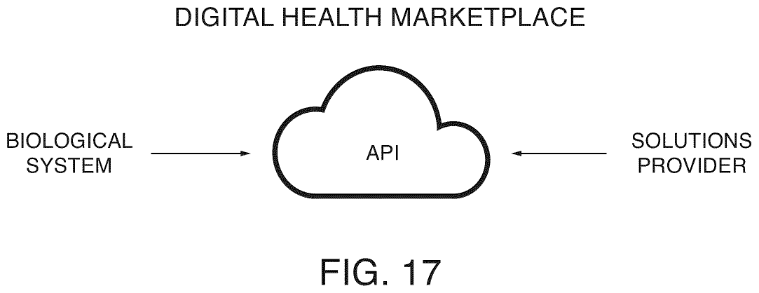

FIG. 18 illustrates, schematically, an embodiment describing how to use work sensor signal to correct heat sensor signal and obtain resting metabolic status.

FIG. 19 illustrates an example of measured data on tracking heat generation and heat elimination during circadian rhythm cycles, generally referred to herein as “thermal fit.”

FIG. 20 illustrates that, although there is diversity in the details of circadian rhythms, all healthy people show a general alignment between work and heat elimination; it also illustrates that disease, injury, and aging impair the body's capacity to eliminate heat, such that a thermal backlog may accumulate throughout the day, and a metric called thermal alignment quantifies alignment/backlog and is highly sensitive to changes in health.

FIG. 21 illustrates thermal misalignment through thermal backlog in unhealthy subjects and compares this to thermal alignment in a healthy subject.

FIG. 22 illustrates the effect of treatment, showing thermal backlog in an unhealthy subject, and the effect of treatment to achieve thermal realignment.

FIG. 23 illustrates a correlation of treatment to improved activity by reference to “thermal fit.”

FIG. 24 illustrates that “thermal fit” is a new and scalable vital sign of energy metabolism.

FIG. 25 shows “thermal fit” in a healthy subject versus in an unhealthy subject.

FIG. 26 illustrates how the “thermal fit” indicator can be used for pre-symptomatic diagnosis and timely treatment.

FIG. 27 shows an example of a heat signature for a healthy person, showing data recorded over 48 hours.

FIG. 28 illustrates an example of a decision support system which can be utilized by the embodiment of using work sensor signal to correct heat sensor signal and obtain resting metabolic status as illustrated in FIG. 18.

### DETAILED DESCRIPTION OF THE PREFERRED EMBODIMENTS

As noted above, a basic tool of modern biological sciences is organic chemistry, the study of carbon and its bonds with other key atoms. This is, at least in part, because the genetic, signaling, and structural molecules of living systems consist largely of carbon atoms. Carbon atoms, however rank only third in abundance in the human body, at around 12%. Despite carbon bonds' great diversity, carbon bonds are neither the most numerous in the body, nor are they involved in most energy transfer processes in biology. In contrast, the most abundant elements in biological systems are hydrogen and oxygen. Hydrogen and oxygen atoms constitute water, as well as other key molecules (for example, hydroxide, hydrogen ion, superoxide, hydronium ion, hydrogen peroxide, trioxidane, etc.). Such hydrogen and oxygen-containing molecules form a vast network of constantly fluctuating hydrogen bonds which absorb, hold, transmit, balance, moderate, and reemit all energy that passes through a biological system. Together, these hydrogen and oxygen atoms constitute over 85% of the human body and participate in all of its processes.

The disclosed technology provides in certain embodiments for the direct or indirect measurement of the intrinsic or emergent, irrespective of origin, transient or permanent, thermodynamic, electrochemical, acoustic, structural, chemical or biological properties of water alone or water in combination with other water molecules or ionic or radical forms of oxygen or hydrogen and oxygen, and combinations of these with carbon or non-carbon substances—for example, elements, ions, molecules, cofactors, minerals, salts, polymorphs or mixtures. We use the terms “water*” and “water star” to refer to water and any of the aforementioned entities associated with water, either alone or in combination.

The disclosed technology is based upon, and involves useful application of the insight that water*, in contrast to other solvent systems, particularly as a component of a biological system, exhibit at least the following physical properties that can be related, directly or indirectly, to one or more states or function of a biological system. As water is central to the function of every chemical or physical process in all biological systems at all scales (for example, whole body, cell, tissue, organ, etc.) and the availability of sufficient associated water* is thus important to the function of a biological system it is therefore useful to select and utilize sensors which measure directly and/or indirectly the properties of water* for quantifying and learning at least the following operational properties of a biological system,
1. High Heat Capacity: The relatively high heat capacity of water and aqueous systems provides thermal stability to the milieu interior of a biologic system. In contrast, other typical or abundant solvents, have substantially have less than half the heat capacity of water.
2. Incompressibility: The relative incompressibility of water and aqueous systems provides a physical basis for structural stability to a biological system. In contrast, other typical or abundant solvents are substantially more compressible and therefore susceptible to structural damage.
3. Large Thermal Diffusivity: Water and aqueous systems have anomalously high thermal diffusivity, comparable to that of a solid. This enables the deleterious internal temperature variations around active organelles to be minimal. In contrast, other typical or abundant solvents are substantially less efficient at distributing energy and would have much larger temperature gradients around metabolizing centers potentially resulting in structural degradation.
4. Large Infrared Absorption Band: Metabolism makes and breaks carbon bonds in specific ways to build organic structures. The waste heat from these processes is efficiently captured by the broad infrared absorption band of water. In contrast, other typical or abundant solvents are substantially less efficient at capturing heat. Efficient capture of heat is also essential to rapid enzyme kinetics.

As will be appreciated by a person of ordinary skill in the art, there exist other properties of water*, be these distinct or related to the above-described properties of water*, that that can be related, directly or indirectly, to one or more states of a biological system. Certain of those other properties of water* are described herein.

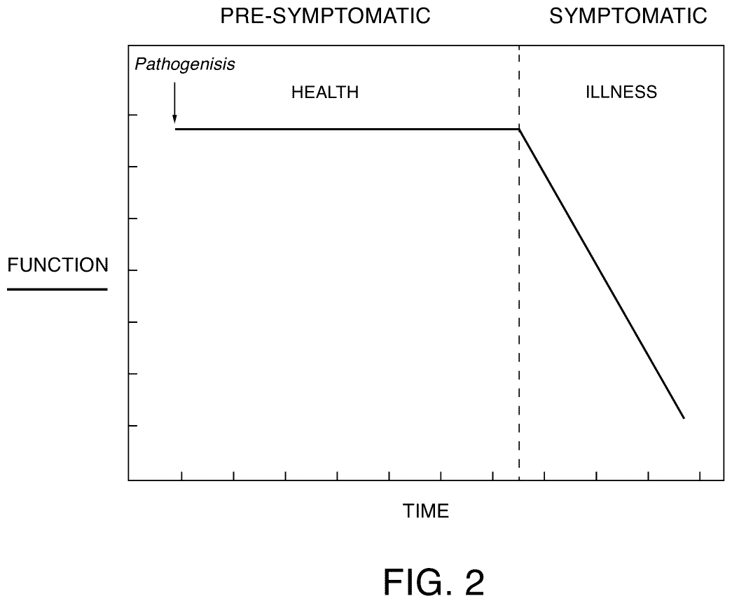

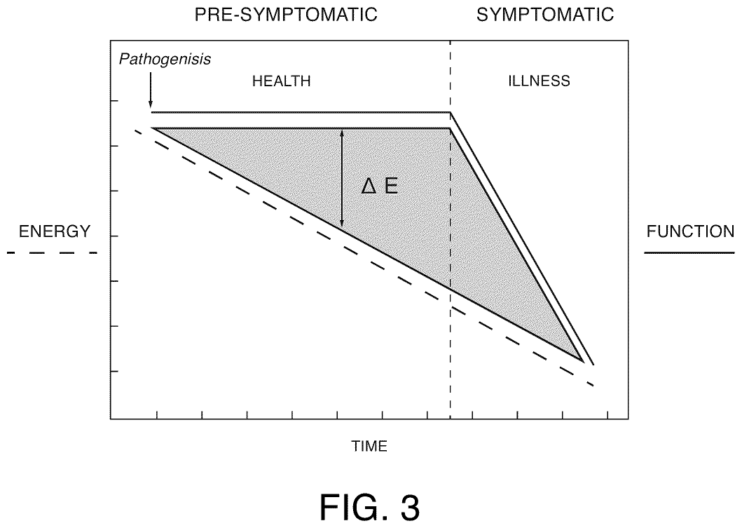

“Health capacity,” as used herein, refers to an inherent property of emergent systems, including biological systems, such as organisms. For example, as shown in FIGS. 2 and 3, which illustrates how “health capacity” may be envisioned as an inherent property of emergent systems. In the context of biology, health capacity may be considered, for example, to be the energy and information contained in a living system or an organism that enables persistence or function or health of that system or organism. When health capacity is high, the system may be defined, for example, as fit or, more specifically, healthy or, even more specifically, free of disease or free or infection. When health capacity is low, the system may be defined as frail or, more specifically, susceptible to disease or to infection or injury, or even more specifically, infected or diseased. Disease may be brought about by a reduction in health capacity, or disease may, itself, through loss of function cause a reduction in health capacity.

To quantitatively understand the relationships among water* and emergent properties, within the context of the disclosed technology, the First Law of Thermodynamics may be applied to sum up heat and work to obtain an “energy budget.” While “energy expenditures” support “metabolic tasks” underpinning health capacity, the expressions of energy in biology are largely embodied in water*. Specifically, a connection between water*, emergence and health capacity lies in the ability of water* to maintain and exploit physical chemical gradients to do work, as exemplified in Equation 1:

water*+gradients=work=metabolic task(s)→health capacity EQUATION 1:

Health capacity, thus, may be understood as involving work to perform a metabolic task and therefore requires an energy expenditure. A stable source of free energy is required to balance the energy expenditure. Biological systems store free energy in the form of physical and chemical gradients; the magnitude of available energy is proportional to the scale of the gradient and the kinetics of the processes that control the relaxation of the gradient. Aqueous solutions have the unique ability to support a large diversity of gradient types (thermal, pH, osmotic, redox, etc.) and the transport properties of water allows for tunable kinetics of energy release. Gradients in aqueous solutions are often stabilized around biological structures, cell and organelle membranes, tissue and organ boundaries and skin. In fact, the structural motifs of biology (cells and fractalized conduits) allow for gradients to be ubiquitous and internal rather than monolithic and external. Therefore, the function of organic chemistry may be viewed as to harness and optimize the health capacity of organized gradients in aqueous solution and distribute them optimally through a biological system. Nonetheless, health capacity is or may be a pre-organic phenomenon which arises from the inherent property of aqueous solutions to hold and release energy stored in physical chemical gradients. Examples of gradients in the biological systems which are connected to health capacity include: temperature gradient at the skin surface controllable by vasodilation allows for metabolic flexibility and homeothermicity; proton gradient at the mitochondrial membrane as a primary power source in eukaryotic cells; hydrostatic/oncotic pressure balance as key driver of nutrient and water partitioning; and the Bohr and the Haldane effects as capacity multipliers in the circulatory/respiratory system.

Health capacity may be understood through the interrelated roles played by water, in living systems, in heat transfer (especially heat elimination) generally, and in the periodicity of heat transfer (especially heat elimination). The technology disclosed in the present disclosure permits insights into the unique signature of one or more living systems, or a population of living systems, and thus into the health capacity of such a living system. The technology permits, for example, the measurement of the emergent property of heat elimination, measured either or both as an absolute value or as a periodic value. Absolute values of heat elimination may be indicative, or reflective, of energy expenditure. Absolute values of heat elimination may be indicative, or reflective, of metabolic rate insight. The periodicity of heat elimination may provide further insight into health capacity. The periodicity of heat elimination may be of a circadian periodicity (that is, on a cycle of from about 23 hour to a cyle of about 25 hour cycle), may be of a cycle shorter than circadian periodicity (for example, on a cycle of about 12, about 14, about 16, about 18, or about 20 hours), or may be longer than circadian (for example, every 2, 3, 4, 5, or 6 days, every week, or on a roughly monthly or 28-day, lunar, or annual cycle). Any such periodicity of heat elimination may serve as, or be reflective of, a unique signature a living system having a standard, acceptable health capacity. Deviations from that standard, acceptable periodicity of heat capacity may indicate, or otherwise reflect, sub-standard or unacceptable health capacity.

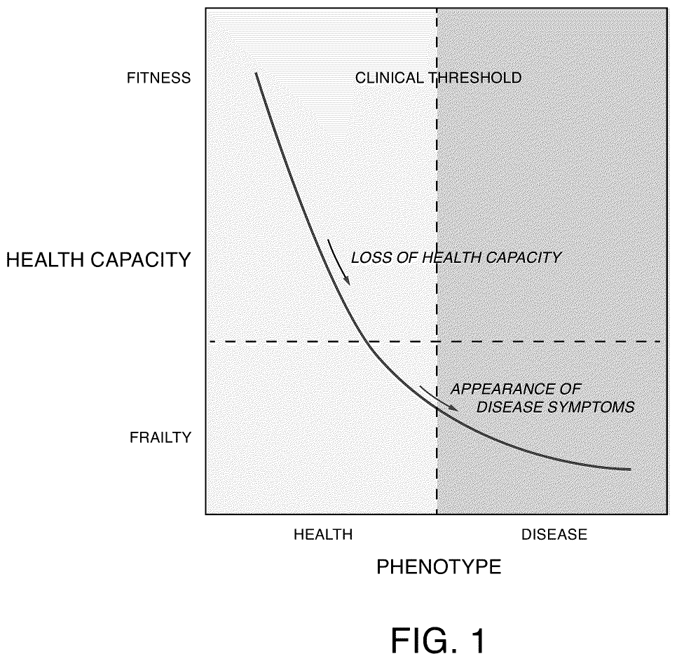

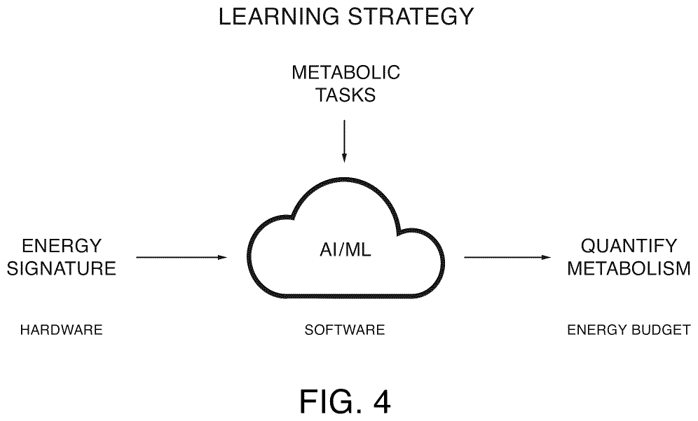

With respect to health capacity, “pre-symptomatic disease,” as used herein, refers to a process wherein health capacity is depleted by some metabolic task which is compensating of an environmental stress, the end result of which is disease and loss of function. This reduced health capacity is detectable as an anomalous energy expenditure associated with compensation or as some more general anomalous energy signature. Such a state of low or lowered health capacity places an individual at higher risk for loss of function generally, not merely from the initial stress or, more specifically, in a state of general susceptibility to disease or infection. Further, “symptomatic disease,” as used herein, refers to a state of impaired function or, more specifically, a disease state or a state in which an organism is, for example, infected by a viral pathogen. Current methodology, exemplified by the precision medicine paradigm, employs symptoms as indicators of disease (as illustrated in FIGS. 1 and 2). The presently disclosed technology provides systems, devices, and methods for quantifying more fulsome merits of health, exemplified by the measurement and assessment of “health capacity,” as either a static or dynamic measurement, to enable assessment of and increases in health capacity, and to identify pre-symptomatic changes in health capacity to permit early detection of disease or, more specifically, for example, infection (as illustrated in, for example FIGS. 2 and 3). An embodiment describing a learning strategy using measurement of energy and annotations to learn rules of health capacity is illustrated in FIG. 4.

Health, in one embodiment, is related to the ability of a biological system or organism to successfully adapt to a variety of challenges without significant loss of function. Physiologists, for example, may describe health as the sufficiency of a form of stored energy they call physiologic reserve—the ability of a human to positively respond to a stress. Physicists, as another example, may describe health as a capacity to incorporate, transform, and dissipate energy to persist. Cell biologists, as another example, may describe health as the baseline state of homeostasis—the ability of a cell or tissue to auto-regulate. Biochemists, as another example, may describe health as the control of anabolic and catabolic reactions in a metabolic network critical to biological function.

Each of these above-described perspectives or understandings of health is appropriate, in its context, be that the perspective of the physiologist, physicist, cell biologist, or biochemist, and contributes to the application of the disclosed technology. More specifically, the disclosed technology reconciles the above perspectives and understandings, and applies a novel conceptualization of health as high health capacity simultaneous with absence of disease-functional and fit. In one embodiment, health capacity, as illustrated in FIG. 1, reveals how health may be quantified by measuring physical properties of homeostasis. That is, health is quantifiable as the health capacity of a biological system. Other aspects of the disclosed technology relate to developing techniques that accurately measure an array of metrics of the state of the biological system, including, for example, the health the biological system. These techniques obtain and process information on an actionable time scale, at sufficiently high resolution.

Other aspects of the disclosed technology include: quantifying emergent properties of biological systems; automating new measures and metrics of emergent properties of biological systems to improve life; action plans to acquire, maintain, and promote health; allowing for faster learning of complex biology from new health accuracy data streams; allowing frictionless collection, analysis, and decision support of health in subjects; developing a scalable solution to disease interception; reducing false-positives in diagnostics; and finding causes of diseases. In some embodiments of the disclosed technology, properties of living systems and their physical-chemical properties are measured; these properties capture the emergent properties of living systems. In other embodiments, the disclosed technology is focused on emergent properties that gave rise to the boundary between physical and biological systems. The disclosed technology is thus applicable to synthetic biology and artificial biological systems, for example, protocells or industrial biology systems.

In another aspect, the disclosed technology relates to methods for measuring an array of health metrics from a subject. In some embodiments, the methods are integrated into an internet of things (IoT) architecture and infrastructure. In some embodiments, the methods measure features of metabolism at the scale of cells. These measurements may be intermitted, regular, or continuous. In some embodiments, the methods further comprise streaming arrays of such metrics into the Cloud, applying machine learning to “quantifying metabolism” and detecting changes before symptoms of diseases occur. In some embodiments, the methods non-invasively measure discrete physical parameters of biological systems at resolution and frequency that in real-time measure cellular physiology and homeostasis. In some embodiments, the methods define and quantify a quantity known as the health capacity of a subject, relating to the resilience of the subject's homeostasis, based on measured physical properties, energy and information of the subject.

The disclosed technology provides actionable information permitting the improvement of health capacity in a biological system. In preferred embodiment, individuals gain access to metrics, not previously accessible, of their own health. Such actionability permits the observations of responses to particular stimuli in a specific biological system and permits alternation or influence of future stimuli. For example, an individual or health care professions may use the disclosed technology to alter the health of a biological system, for example, a patient or the individual himself or herself. It also permits a better understanding of the most influential determinants of health, for example, sleep patterns and duration, nutrition (including diet, supplements, medications, etc.), exercise (neuromuscular inputs, type, duration, periodicity, etc.) and other lifestyle decisions or detractors (e.g., pollution) that impact health. In a preferred embodiment, the disclosed technology permits an individual to be an efficient agent of his own or her own health.

The disclosed technology provides automated and automatable methods, thus addressing a major sources of healthcare inefficiency, manual testing and interpretation. Current healthcare testing is generally centralized, and the interpretation of such testing is generally performed manually by healthcare professions. Both of these tasks are expensive and inefficient. The disclosed technology permits the digitalization of health data associated with health capacity. Such digitization affords the ability to apply all the benefits of the existing IoT ecosystem and Cloud computing.

The disclosed technology provides scientists and healthcare professions with systems and methods to optimize health by improving health capacity. Rather than viewing health as an elusive concept, the current technology permits it to be viewed as a system with quantifiable, particularized or modularized measurable tasks or targets.

The disclosed technology permits the quantification of health viewed an as health capacity, and the detection of its change over time, including changes in magnitude or frequency of circadian or other periodic components. Rather than the current paradigm, viewing the absence of health as the presence of symptomatic disease, the disclosed technology provides for the substantially continuous measurement of health, quantification of its decline, and detection its change prior to the onset of symptomatic disease. Thus, the disclosed technology permits individuals, public health and/or medical professionals to take corrective action with more accurate, reliable information to forestall the onset of disease, decrease morbidity, decrease mortality, and lower the cost of care.

The disclosed technology can also increase the efficiency of currently available precision medicine tools by reducing the false positive rate of discovery, by employing simple, fundamental and translatable metrics of energy usage—an “energy signature,” and thereby reverse the trend of inefficiency according to Eroom's Law.

In some aspects, the disclosed technology relates to methods of obtaining physical, chemical, and biological measurements of emergent properties of both biological and non-biological systems; and a system or technology platform that processes, stores, transfers, analyzes, compiles, distributes, and displays emergent property measurement data to quantify, predict, control, maximize, design, and engineer complex adaptive biological and non-biological systems.

In some embodiments, a biological system could be a eukaryotic, prokaryotic, or archaea organism such as a bacteria, gamete or red blood cell, white blood cell, a cell derived from a tissue or organ such as a myocyte, or a simple or complex multicellular organism such as an apple or human, or an engineered cell, and/or a collection thereof to form an interactive ecosystem in conjunction with non-biological essential chemical materials or energy sources.

In some embodiments, a non-biological system could be an engineered non-living system analogous to a biological system but devoid of genetic material—a protocell—or any other engineered or biomimicry-based designed system or semi-synthetic system.

In some embodiments, the disclosed technology further relates to the use of such measures to gauge their state of existence and their ability to persist in biological and non-biological systems. The ability of a biological or non-biological system to exist or persist relates to the system's “health capacity.”

Advantages of the disclosed technology include its ability to quantify, predict, control, maximize, engineer, and design a biological or non-biological system based on measurement of physical, chemical, and biological parameters that relate to the property of resilience (adaptivity) as indicated primarily by its ability to persist or achieve some core function capability.

In some embodiments, the disclosed technology further relates to a technology platform and methods for obtaining physical, chemical, and biological parameters include wearable, implanted, embedded, or otherwise-coupled devices. Once such measurements are implemented, they can be stored, quantified, analyzed, compiled, distributed, and displayed to quantify, predict, control, maximize, engineer, and design a biological system. Measures of health capacity can also be analyzed in conjunction with other measures that are not those of emergent properties to improve upon the ability of the measurement technology and learning platform to quantify, predict, control, maximize, engineer, and design a biological or non-biological system.

In the example of single-celled eukaryotic or prokaryotic or archaea organisms, the disclosed technology would allow for the quantification, prediction, and maximization of function. In the case, for example, of a gamete, the disclosed technology would allow for the quantification of health capacity to maximize fertility for in vivo or in vitro fertilization. In bacteria, yeast or human cells, the disclosed technology could be used for the quantification of health capacity of, in the case of industrial or synthetic biology, the synthesis of molecules. In the case of non-pathogenic enteric bacteria, the disclosed technology could be used to maximize microbiome function.

In the example of multi-celled eukaryotic organisms, the disclosed technology would allow for the quantification, prediction, and maximization of function. For example, in humans the disclosed technology would enable the measurement and maximization of health and the diagnosis and/or pre-symptomatic diagnosis of disease, and the designs of methods for disease treatment or interception, where disease could be infectious, cancer, toxic, iatrogenic, or metabolic, and where health determinants are food/nutrition, sleep, exercise, neuromuscular activation, and changes to lifestyle such as smoking, inactivity, addiction.

In certain embodiments, the advantages of the disclosed technology can further include its applications in non-human multi-celled eukaryotic organisms. For example, in agricultural systems, such as farming of plants and animals. In these biological systems the disclosed technology would enable the maximization of food substance production and/or mitigation of abiotic or biotic stress.

In certain embodiments, the advantages of the disclosed technology can further include its application in simple ecosystems of species and chemical resources. For example, in biological systems of two or more species and one or more resources, the disclosed technology would enable their mutual maximization of functional interdependence. Where one of the species was a human, for example, the technology would enable the maximization of human functional parameters such as sleep, activity, physical or cognitive performance and disease prevention.

In certain embodiments, the advantages of the disclosed technology can further include its applications in complex ecosystems. For example, in biological systems of many species and many resources, the disclosed technology would enable their mutual maximization of functional interdependence. Where the complex system was a farm the disclosed technology would enable maximization of an ecosystem's function such as sustainability.

In certain embodiments, the advantages of the disclosed technology can further include its applications in synthetic biology, for example in eukaryotic and prokaryotic cells. In these biological systems, the disclosed technology would enable design and engineering to maximize function; for example, the synthesis of a protein, lipid, or small molecule or the capability to maintain viability under condition of abiotic or biotic stress.

In certain embodiments, the advantages of the disclosed technology can further include its applications in biometrics. For example, the disclosed technology would enable the identification of a biological system independent of or in conjunction with genetic material.

In certain embodiments, the advantages of the disclosed technology can further include its applications in non-biological systems. For example, the disclosed technology would enable the design and engineering of a non-living system analogous to a biological system but devoid of genetic material—a “protocell.” Such protocell could be used for learning and/or doing work, where work may be understood to mean any output that is not merely heat production.

In certain embodiments, the advantages of the disclosed technology can further include its application in biological systems to quantify biological time-“life span.” For example, the disclosed technology could be used to calculate the theoretical and actual life span(s) of a biological system and be used in conjunction with the aforementioned uses to maximize function or life-span.

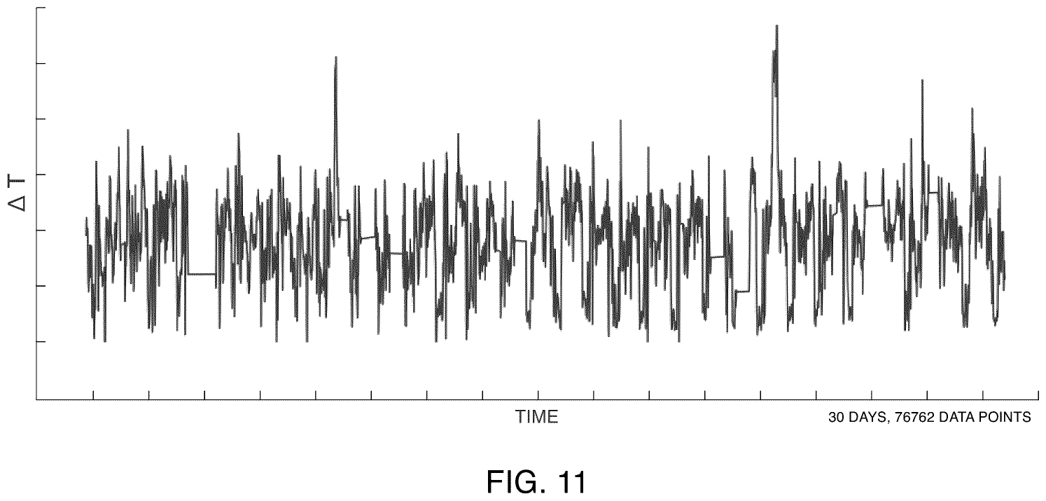

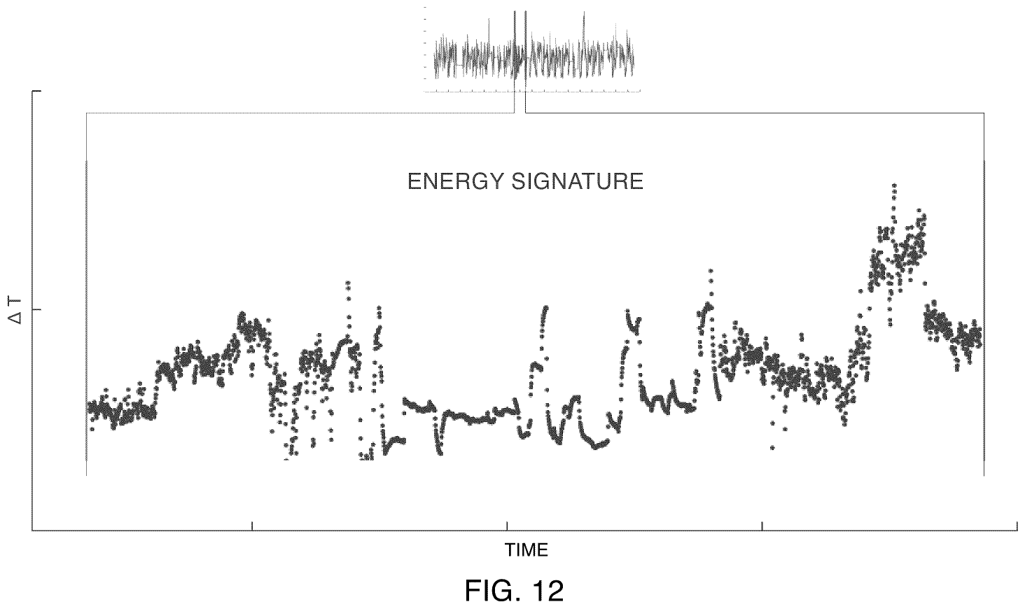

In another aspect, the disclosed technology relates to a wearable device comprising an array of sensors that record health metrics. In some embodiments, the wearable device continuously records select “energy signatures” (as illustrated in, for example, FIGS. 11 and 12) metrics or indicators of health for the subject. In some embodiments, the wearable device captures emergent-derived complexity at the scale of cell physiology. In some embodiments, the wearable device requires low cost and low power, enabling accessibility and continuous data capture in real-time. In some embodiments, the wearable device comprises a multi-modality sensor system that measures electrochemical, mechanical, structural, thermal, and/or energetic properties reflective of homeostasis and cell physiology. In some embodiments, the wearable device comprises from about four sensors to about twelve sensors. In some embodiments, the wearable device comprises from about five, six, seven, eight, nine, ten, or eleven sensors.

In some embodiments, the wearable device measures skin and ambient temperature across an array of locations on the subject as a function of time. Changes in temperature, over fixed time intervals such as an hour, a twelve-hour period, a 24-hour period, a 48-hour period, may quantify heat transfer and thus reflect, for example, an intrinsic metabolic rate or changes in such a rate. In some embodiments, the wearable device measures relative humidity and barometric pressure at relevant locations on the subject as a function of time; changes in humidity and barometric pressure may affect heat transfer and its quantification. In some embodiments, the wearable device measures electrochemical properties such as impedance across an array of locations on the subject as a function of time; changes in electrochemical properties may quantify electrolytes and may reflect water flux. In some embodiment, the wearable device measures cellular mechanics across an array of locations on the subject as a function of time; changes in cellular structure, for example, may quantify motion that reflect structural dynamics. In some embodiments, the wearable device measures at least two, three, four, five, six or more such metrics simultaneously.

In another aspect, the disclosed technology relates to a scalable technology platform that automates the regular, intermittent, or continuous collection and interpretation of energy signature data streams from a plurality of subjects including other data streams or annotations of “metabolic tasks.” In some embodiments, the data collection and interpretation are based on at least two, three, four, five, six or more of the above metrics simultaneously. In some embodiments, the data are compressed and/or encrypted. In some embodiments, the scalable technology platform further comprises an application programming interface (API). In some embodiments, the data are compressed and/or encrypted and stored in the cloud. In some embodiments, the scalable technology platform further provides tools for optimizing subjects' health and for chronic disease management. In some embodiments, the scalable technology platform further comprises a suite of machine learning processes used to iteratively improve the technology platform. Advantages of the scalable technology platform include the ability to: quantify health; develop objective tools for its optimization; detect disease pre-symptomatically; and intercept diseases.

In some embodiments, the scalable technology platform further provides tools to objectively quantify subjects' health. The platform may correlate or predict the impacts of one or more of food, nutrition, exercise, activity, sleep, lifestyle, genome, aging, community, and/or fetal-maternal well-being to health. In some embodiments, the scalable technology platform defines and quantifies the frailty index of a subject or an elderly population, which relates to the generalized diminution of physical capacity and resilience as a result of aging. In some embodiments, the frailty index relates to muscle wasting, left-right asymmetry in muscle performance, inflammation associated with chronic disease, cardiovascular insufficiency, and decreased mental capacity. In some embodiments, the scalable technology platform further provides tools to detect pre-symptomatically, track and intercept events relating to infection, sepsis, rehabilitation, and/or chronic disease.

In some embodiments, the scalable technology platform further provides automatically generated treatment options pre-symptomatically or at an early disease stage, e.g., earlier institution of antibiotics and supportive therapy. In some embodiments, the scalable technology platform further provides automatically generated recommendations to improve a subject's health state.

Described herein are systems for quantifying a health capacity of a biological system, comprising: at least one sensor configured to measure an emergent factor of the biological system and generate measured data based on the emergent factor; and a processing system comprising a processor and an interface for receiving the measured data from the at least one sensor and determining one or more factors that quantify the health capacity of the biological system based on the measured data. In the system, the processor may compute a solution for maximizing the health capacity of the biological system according to machine readable instructions. The biological system may be an organism. The biological system may be selected from an animal, a plant, and a single cell organism. The biological system may be an industrial biology system or a synthetic biology system. In a preferred embodiment, the organism is a human. The system may further comprise a storage component in communication with the processing system and for storing the measured data. The measured data may be an energy budget of the biological system. The system may be structured such that the processing system comprises a plurality of transmitters configured to transmit the measured data as data streams optimized with respect to properties of the at least one sensor and the emergent factors to be reported. In a preferred embodiment, the processor makes pre-symptomatic detection of a disease state of the biological system based on the health metrics, according to machine readable instructions. The processor may, alternatively, make pre-symptomatic detection of the disease state of the biological system using a supervised learning algorithm according to machine readable instructions with a collection of health metrics reported from a plurality of other objects. The disease state may be, in certain embodiments, selected from aging, sepsis, cardiovascular disease, and infectious disease. If the disease state is an infectious disease, the infectious disease may be caused by a viral infection, and the viral infection may be selected from a respiratory infection, a gastrointestinal tract infection, a liver infection, a nervous system infection, and a skin infection, or a coronavirus, such as, for example, COVID-19. The at least one sensor of the system is, preferably, a thermodynamic sensor, but may also be an electrochemical sensor, a structural sensor, a tensile sensor, a motion sensor, other known sensors, or a combination thereof. The at least one sensor may, in certain embodiments, comprise a plurality of wearable devices for sensing data comprising at least one of heat flux data, calorimetry data, osmometry data, and physiometry data, and may be a wearable device or an implanted device. The interface may be configured to transmit the measured data via wireless communication. The processing system may, in certain embodiments, further comprise: an application program interface that controls storage of the measured data, access to the measured data, security configurations, user inputs, and output of any results.

Also disclosed is a system for quantifying a health capacity of a biological system, comprising: a plurality of measuring devices, wherein at least one measuring device measures a thermodynamic property of the biological system. In some embodiments, the system includes a solution for intercepting a disease state.

Also disclosed is a method for quantifying a health capacity of a biological system, comprising: sensing at least one emergent factors of the biological system, generating measured data relating to the at least one emergent factor, and determining, based on the measured data, one or more stimuli that influence the health capacity of the biological system. The method may, in certain embodiments, further comprise generating a solution for maximizing the health capacity by modifying one or more stimuli that influence the health capacity of the biological system, wherein the stimuli are selected from sleep patterns, sleep durations, nutritional intakes, and exercise regimens. In certain embodiments, the measured data may comprise surface temperature and physical activity of the biological system over time. The method may further comprise: estimating heat elimination of the biological system over time based on differential surface temperature; estimating heat production of the biological system over time based on physical activity; and estimating a basal metabolic status of the biological system based on temporal alignment of heat elimination and heat production. The method may further comprise: obtaining a quasiperiodic rhythm of the biological system based on the measured data, wherein the quasiperiodic rhythm is of seconds-timescale, of minutes-timescale, ultradian, circadian, circalunar, or of yearly timescale. In certain embodiments, the method may further comprise: obtaining a variability of the quasiperiodic rhythm across a predetermined amount of time; and determining the health capacity based on the variability of the quasiperiodic rhythm. The method may further comprise: estimating heat elimination of the biological system over time based on differential surface temperature; estimating heat production of the biological system over time based on physical activity; estimating a basal metabolic status of the biological system based on temporal alignment of heat elimination and heat production; and determining the health capacity by applying a time-dependent function to the estimated basal metabolic status, wherein the time-dependent function is derived from the quasiperiodic rhythm of the biological system.

Also disclosed is a system for determining an energy signature of a non-biological system, comprising: at least one sensor configured to measure an emergent factor of the system and generate measured data based on the emergent factor; and a processing system comprising a processor and an interface for receiving the measured data from the at least one sensor and determining one or more factors that quantify an energy budget of the non-biological system based on the measured data. In certain embodiments, this system may comprise at least one thermodynamic sensor and at least one motion sensor. The at least one thermodynamic sensor may comprise a plurality of wearable devices for sensing surface temperature of the biological system over time, and wherein the at least one motion sensor comprises at least one accelerometer for sensing physical activity of the biological system over time.

In the system, the processing system may be further configured to analyze a quasiperiodic rhythm and activity levels of the biological system based on the measured data, wherein the quasiperiodic rhythm is of seconds-timescale, of minutes-timescale, ultradian, circadian, circalunar, or of yearly timescale. Also, the processing system may be further configured to actuate the sensors based on the analyzed quasiperiodic rhythm and activity levels of the biological system. In certain embodiments of the method, the measured data may comprise exhaust streams of the biological system. In certain embodiments, the exhaust streams may comprise heat, one or more low energy chemical species, or any combination thereof. In certain embodiments, the measured data may comprise total energy expenditure of the biological system in real time. In certain embodiments, the method further comprises: analyzing functional aspects of thermoregulation in the biological system based on the measured data. In certain embodiments, the method further comprises: generating and outputting indicators for understanding, improving, modulating, repurposing, or any combination thereof, one or more functions of the biological system. In certain embodiments of the method, the indicators are used to manage weight, blood pressure, circadian rhythm, sleep quality, sleep duration, or any combination thereof, of the biological system.

In certain embodiments of the system, the system comprises at least one heat sensor and at least one chemical sensor configured to measure exhaust streams of the biological system. The exhaust streams may comprise heat, one or more low energy chemical species, or any combination thereof. The sensors in certain embodiments are configured to directly measure total energy expenditure of the biological system in real time. In certain embodiments, the processing system is configured to automatically analyze functional aspects of thermoregulation in the biological system based on the measured data. In certain embodiments, the processing system is further configured to automatically generate and output indicators for understanding, improving, modulating, repurposing, or any combination thereof, one or more functions of the biological system. In certain embodiments, the indicators are used to manage weight, blood pressure, circadian rhythm, sleep quality, sleep duration, or any combination thereof, of the biological system. In certain embodiments, at least one indicator suggests automatically administering suitable amounts of one or more: decoupling agents, modulators of the oxidative phosphorylation pathway, modulators of transmembrane ionic gradients, or any combination thereof. In certain embodiments, at least one indicator suggests automatic control of the external environment to influence the biological system's thermoregulatory functions or physiological aspects relating to thermoregulation.

In certain embodiments of the system, the biological system's thermoregulatory functions or physiological aspects relating to thermoregulation comprise cardiovascular parameters, circadian parameters, cognitive parameters, emotional parameters, or any combination thereof. In certain embodiments, automatic control of the external environment comprises adjusting interior air temperature, pressure, humidity, or any combination thereof. In certain embodiments, automatic control of the external environment comprises providing auditory stimuli, olfactory stimuli, visual stimuli, or any combination thereof.

In certain embodiments of the system, at least one indicator makes automatic recommendation to the biological system to take predefined actions. In certain embodiments, the biological system is a human being, and wherein the predefined actions comprise: change clothes, go inside, go outside, eat a specific food, drink water, do certain exercises, go to sleep, or any combination thereof. In certain embodiments, the sensors are configured to directly measure total energy expenditure of the biological system in real time. In certain embodiments, the processing system is configured to automatically analyze emergent properties of thermoregulation in the biological system based on the measured data.

In certain embodiments, the processing system may be further configured to generate and output indicators for understanding, improving, modulating, repurposing, or any combination thereof, one or more emergent properties of the biological system. In certain embodiments, the indicators are used to manage weight, blood pressure, circadian rhythm, or any combination thereof, of the biological system. In certain embodiments, the measured data comprises heat flux data.

In certain embodiments, at least one health capacity is a basal metabolic status, and at least one emergent factor is the temporal alignment of heat production and heat elimination. In certain embodiments, the temporal alignment is related to at least one quasiperiodic rhythm of the biological system. The at least one quasiperiodic rhythm may be a circadian rhythm. In certain embodiments, the method further comprised the step of generating and outputting at least one indicator for improving or modulating the temporal alignment of heat production and heat elimination of the biological system. In certain embodiments, the indicator suggests to the biological system to perform to at least one predefined action selected from the group of actions comprising: change clothes, go inside, go outside, eat a specific food, drink a specified beverage, perform certain exercises, go to sleep, or any combination thereof. In certain embodiments, the method further comprises the step of recommending at least one predetermined action to improve or modulate the temporal alignment of heat production and heat elimination of the biological system. The action may manage circadian rhythm.

In certain embodiments, the measured data comprises heat flux data, at least one health capacity is a basal metabolic status, and at least one emergent factor is the temporal alignment of heat production and heat elimination of the biological system. The temporal alignment is related to at least one quasiperiodic rhythm of the biological system. The quasiperiodic rhythm is a circadian rhythm. In certain embodiments, the processing system is further configured to generate and output at least one indicator for improving or modulating the temporal alignment of heat production and heat elimination of the biological system. In certain embodiments, the indicator suggests to the biological system to perform to at least one predefined action selected from the group of actions comprises: change clothes, go inside, go outside, eat a specific food, drink a specified beverage, perform certain exercises, go to sleep, or any combination thereof. In certain embodiments, the at least one indicator suggests administering suitable amounts of one or more of the following: a decoupling agent, a modulator of the oxidative phosphorylation pathway, a modulator of transmembrane ionic gradients, or any combination thereof.

Also disclosed is a system for quantifying and improving a metabolic status of a human, comprising: at least one wearable thermodynamic sensor configured to: measure an emergent factor of the human, wherein the emergent factor is the temporal alignment of heat production and heat elimination of the human, the temporal alignment relating to the circadian rhythm of the human, and based on the emergent factor, generate measured data comprising heat flux data over time; and a processing system comprising a processor and an interface, the processing system configured to: receive the measured data from the at least one wearable thermodynamic sensor, based on the measured data, quantify a metabolic status of the human relating to the heat production and heat elimination, based on the measured data, determine one or more stimuli that influence the metabolic status of the human, compute a solution for maximizing the metabolic status of the human, and generate and output at least one indicator for improving the metabolic status of the human by modulating the heat production and heat elimination of the human.

Also disclosed is a method for quantifying and improving a metabolic status of a human, comprising: sensing at least one emergent factors of the human, wherein the emergent factor is the temporal alignment of heat production and heat elimination of the human, the temporal alignment relating to the circadian rhythm of the human; generating measured data relating to the at least one emergent factor, the measured data comprising heat flux data over time; based on the measured data, quantifying a metabolic status of the human relating to the heat production and heat elimination; based on the measured data, determining one or more stimuli that influence the metabolic status of the human; computing a solution for maximizing the metabolic status of the human; and generating and outputting at least one indicator for improving the metabolic status of the human by modulating the heat production and heat elimination of the human.

In certain embodiments of the system quantifying a metabolic status of the human is based on determining the mean, variance, min and/or max of the heat production and/or heat elimination over at least one circadian cycle. In certain embodiments, quantifying a metabolic status of the human is based on determining the inter-day stability and/or intraday variability of the heat production and/or heat elimination over at least one circadian cycle. In certain embodiments, quantifying a metabolic status of the human is based on comparing the mean, variance, min and/or max of the heat production and/or heat elimination over a particular circadian cycle to the historical value for the human. In certain embodiments, quantifying a metabolic status of the human is based on comparing the inter-day stability and/or intraday variability of the heat production and/or heat elimination over a particular circadian cycle to the historical value for the human.

### Definitions

“Adaptation,” as used herein, refers to the ability of a biological system to change over time in response to its environment. This ability is important to the process of evolution and may determine, in the case of an organism, by heredity, diet, and external factors. Adaptation is an emergent property and is involved in various biological entities and their functions, such as for example learning in the brain, DNA and protein structures and functions, coordination of organelle functions and homeostasis, feedback control of the transcriptional and translational network, molecular interactions, and equilibria in the biosphere. Adaptation is also evolvable and may take various forms. For example, in a protocell, the ability to adapt requires that the physical and chemical properties of the system can: self-organize with a relatively low kinetic barrier to interconvert among multiple functional forms; eliminate heat; and afford communication at a rate roughly equal to the characteristic rates of internal chemistry and external stress.

“Application program interface” (API), as used herein, refers generally to a set of routines, protocols, or tools designed for use in building software applications. In one example, an API may specify how designated software components interact. In one embodiment, APIs are used when programming components of the system to determine, modify, or maximize health capacity. APIs may be software tool sets that are used to create and implement software and/or instructions to modify sensors or, alternatively, determine health capacity based on data provided by sensors.

“Biological system,” as used herein, refers to any network of biologically relevant entities. In its broadest aspect, a biological system is any network of chemical reactions which exists as a persistent non-equilibrium configuration by its own devices. Biological systems encompass and span differing scales and are determined based different structures depending on the nature of the biological system. Examples of a biological system on a large scale include, for example, a population of microscopic organisms, a homogenous population of similar organisms living in proximity to one another (for example, a cell culture or a community of humans), a heterogeneous population of organism living in a single ecosystem, biological networks. Examples of biological systems on a smaller scale include an individual organism, for example a single mammal such as a human, an organ or tissue system within such an organism, cellular organelle systems, artificial life systems.

“Calorimeter,” as used herein, refers to a device or system used for calorimetry, the process of measuring the heat of chemical reactions or physical changes as well as heat capacity. Differential scanning calorimeters, isothermal micro calorimeters, titration calorimeters and accelerated rate calorimeters are among the most common types of calorimeter. A simple calorimeter may consist of a thermometer attached to a metal container containing water suspended above a combustion chamber.

“Calorimetry,” as used herein, refers to measuring changes in state variables of a biological system for the purpose of deriving the heat transfer associated with changes of its state due, for example, to chemical reactions, physical changes, or phase transitions under specified constraints. Indirect calorimetry is a process or system for calculating heat that a biological system produces by measuring either its production of carbon dioxide and nitrogen waste (frequently ammonia in aquatic organisms, or urea in terrestrial ones), or from their consumption of oxygen. Heat generated by a biological system may also be measured by direct calorimetry, in which an entire organism or array of organisms is placed inside the calorimeter for the measurement. One example of a widely used calorimeter is a differential scanning calorimeter, which allows thermal data to be obtained on small amounts of material. It involves heating the sample at a controlled rate and recording the heat flow either into or from the specimen.

“Decoupling agents” as used herein, refer to molecules, sometimes referred to as “uncouplers” or “uncoupling agents,” that disrupts oxidative phosphorylation on prokaryotes and mitochondria, or disrupts photophosphorylation in chloroplasts and cyanobacteria. Such molecules are capable of transporting photons through mitochondrial and lipid membranes. Classical decoupling agent have five properties: (1) the release of respiratory control, (2) the substitution of all coupled processes (ATP synthesis, transhydrogenation, reverse electron flow, active transport of cations, etc.) by a cyclic proton transport mediated by the uncoupler; the elimination of protonic and cationic gradients generated across the mitochondrial or prokaryotic membrane; (3) no discrimination in these actions between one coupling site and another, (4) no discrimination between coupled processes driven by electron transfer, and (5) coupled processes driven by ATP hydrolysis. Pseudo-decoupling agents show one or more of these properties, but not all, and thus must be combined with one or more other pseudo-uncouplers to achieve full uncoupling. Examples of decoupling agents include, but are not limited to, 2,4-dinitrophenol (DNP); 2,5-dinitrophenol; 1799 (α,α′-bis(hexafluoracetonyl)-acetone); BAM15, N5,N6-bis(2-fluorophenyl)-[1,2,5]oxadiazolo[3,4-b]pyrazine-5,6-diamine; 2-tert-butyl-4,6-dinitrophenol (Dinoterb); 6-sec-butyl-2,4-dinitrophenol (Dinoseb); C4R1 (a short-chain alkyl derivative of rhodamine 19); carbonyl cyanide phenylhydrazone (CCP); Carbonyl cyanide m-chlorophenyl hydrazone (CCCP); carbonyl cyanide-p-trifluoromethoxyphenyl hydrazone (FCCP); CDE (4β-cinnamoyloxy, 1β,3α-dihydroxyeudesm-7,8-ene); CZ5; desaspidin; dicoumarol; dinitro-ortho-cresol (DNOC); ellipticine; endosidin 9 (ES9); Flufenamic acid; niclosamide ethanolamine (NEN); ppc-1 (a secondary metabolite produced by polysphondylium pseudocandidum); pentachlorophenol (PCP); perfluorotriethylcarbinol; S-13 (5-chloro-3-t-butyl-2′-chloro-4′-nitrosalicylanilide); SF 6847 (3,5-di-t-butyl-4-hydroxybenzylidinemalononitrile); TTFB (4,5,6,7-tetrachloro-2-trifluoromethylbenzimidazole); tyrphostin A9 (SF-6847) (AG17); (+)-usnic acid; XCT-790; mitoFluo (10-[2-(3-hydroxy-6-oxo-xanthen-9-yl)benzoyl]oxydecyl-triphenyl-phosphonium bromide); triclosan (Trichloro-2′-hydroxydiphenyl ether); pyrrolomycin C. The following compounds are examples of known to be pseudo-decoupling agents, to be considered decoupling agents within the meaning of this discloisure: azide; biguanides; bupivacaine; calcimycin (A23187); dodecyltriphenylphosphonium (C12TPP); lasalocid (X537A); long-chain fatty acids, including, for example, linoleic acid; mitoQ10; nigericin; picric acid (2,4,6-trinitrophenol); sodium tetraphenylborate and other salt forms; SR4 (1,3-bis(dichlorophenyl)-urea 13); tetraphenylphosphonium chloride; valinomycin; arsenate.

“Disease,” as used herein, broadly refers to any condition that causes pain, dysfunction, distress, or death to the person afflicted. Thus, disease may include one or more injuries, disabilities, disorders, syndromes, infections, isolated symptoms, deviant behaviors, and atypical variations of structure and function. Diseases may affect biological organisms not only physically, but also mentally. Thus, in the case of a human afflicted with a disease, contracting and living with a disease can alter the affected person's perspective on life. Examples of diseases include those identified and classified on the World Health Organization's 10th revision of the International Statistical Classification of Diseases and Related Health Problems (ICD-10). Such diseases that may affect humans include, infectious and parasitic diseases, neoplasms, diseases of the blood and blood-forming organs, disorders involving the immune mechanism, endocrine diseases, nutritional diseases, metabolic diseases, mental and behavioral disorders, diseases of the nervous system, diseases of the eye and adnexa, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, diseases associated with pregnancy, childbirth and the puerperium, diseases originating in the perinatal period, congenital malformations, deformations and chromosomal abnormalities, as well as injuries, poisoning, and consequences of external causes.

“Data stream,” as used herein, refer to a sequence of digitally encoded coherent signals (packets of data or data packets) used to transmit or receive information that is in the process of being transmitted. A data stream is a set of extracted information from a data provider, and comprises a sequence of ordered lists of elements (representing different signal components) and an associated sequence of timestamps.

“Energy budget,” as used herein, refers generally to a balance sheet of energy income against expenditure. “Energy budget” also refers to a logic, encoded in genetic, epigenetic, or other information-containing structures, which determines the response, in terms of energy expenditures, of a biological system to its circumstance. Energy budget, in its most general sense, is a characterization or quantification of the state of a biological system based on parameters that reflect heat or work in that system. An energy budget is also the logic of energy allocation Energy budget is the subject matter of studies in the field of energetics which deals with the study of energy transfer and transformation from one form to another. The calorie is an example of a basic unit of energy measurement. A biological organism, as one example, especially in a laboratory experiment, is an open thermodynamic system, exchanging energy with its surroundings in at least three ways: heat, work, and the internal energy of biochemical compounds. Energy budget allocations may vary for endotherms and ectotherms. Ectotherms rely on the environment as a heat source while endotherms maintain their body temperature through the regulation of metabolic processes. The heat produced in association with metabolic processes facilitates the active lifestyles of endotherms and their ability to travel far distances over a range of temperatures in the search for food. Ectotherms are limited by the ambient temperature of the environment around them but the lack of substantial metabolic heat production accounts for an energetically inexpensive metabolic rate. The energy demands for ectotherms are generally one tenth of that required for endotherms.

“Energy expenditure,” as used herein, in its most general sense, refers to the measurement of parameters that reflect heat or work in a biological system. “Energy expenditure” also refers to an entropy producing (irreversible) outlay of free energy to power an adaptive task within a biological system. An energy expenditure is largely irreversible (entropy-producing) so it represents energy which cannot be retrieved for other tasks. “Energy homeostasis” or “homeostatic control of energy balance,” as used herein, refers to a biological process that involves the coordinated homeostatic regulation of food intake (energy inflow) and energy expenditure (energy outflow).

“Energy signature,” as used herein, refers to the aggregate pattern of energy expenditures in a biological system which arises from the response of that system's energy budget to a set of circumstantial internal and external stresses. Energy signatures may be used as data streams for machine learning alone or in conjunction with annotations of “Metabolic Tasks” to calculate Energy Budgets which “Quantify Metabolism.”

“Emergent factor” or “emergent property” as used herein, refer to properties of water, water* or of an aqueous system or of a biological system or of a complex adaptive system that may be related to, or form a basis for assessing, the health capacity of the system. Emergent factors or emergent properties may also refer to events, deviations from norm or other time dependent patterns in some measurable parameter of the system. Emergent properties may be observed directly or indirectly. Examples of emergent properties include amphotericity, conductivity, solvation capacity, ion mobility, oxidation-reduction potential, ligand association, hydration, electrolysis, thermal conductivity, heat capacity, thermal absorptivity, adhesion, cohesion, transparency, turbidity, incompressibility, polarity, dipolarity, dipole moment, diamagnetism, voltage range of the liquid phase, temperature range of the liquid phase, abundancy, and speciation, flux of energy, momentum, particles or other substances. Emergent factors also include heat elimination, either as an absolute, static value of heat elimination or as a periodic function, for example a circadian periodicity of heat elimination.

“Frailty index,” as used herein, refers to negative trends or inflections in health capacity.

“Growth,” as used herein, refers to maintenance of a higher rate of anabolism than catabolism. A growing organism increases in size in all of its parts, rather than simply accumulating matter.

“Health,” as used herein, refers to the state of being both fit and free from disease. Health is a self-sustaining state where the absence of disease allows for full function which leads to high health capacity (fitness). High health capacity is in turn, protective against disease. High health capacity and health prolong life.

“Health capacity,” as used herein, is the resilience (adaptivity) of a system expressed primarily by its ability to persist or achieve some core function. The adjectives associated with high or low health capacity are ‘fit’ and ‘frail,’ respectively. Low health capacity-“Frailty”—increases the risk of disease or injury (external stress). Disease diminishes function such that health capacity may be diminished as a result. Therefore, frailty and disease can create a positive feedback toward negative outcome. High health capacity “Fitness”-decreases the risk of injury and increases the ability to perform. Early interception of disease can preserve and maintain health capacity and careful management of health capacity can prevent disease. Maximal health is the stable condition of being both fit and free of disease. Health capacity may be considered a correlation of a state, or energy budget, with a function of a biological system that defines the ability of the biological system to persist. Health capacity may be comprised of several quantities which may not necessarily be compared in the same manner or reduced to a single score. The data analytics may be used to discover the relationship between raw measurements and an abstract health capacity score. Dimensionality reduction or machine learning methods may be used to learn health capacity scores based on the raw measurement time series and to predict adaptation and health outcomes.

“Health capacity rules,” as used herein, refers to the minimal set of attributes of an “energy budget” required to confer “health capacity.”

“Homeostasis,” as used herein, refers to processes and mechanisms for the regulation of the internal environment of a biological system, generally to limit variability of a state and/or maintain the status of a state. An example of a homeostasis at the organismal level is sweating, which serves to reduce temperature. An example of homeostasis at the biochemical and cellular level is redox control and its regulation of metabolism.

“Infection,” as used herein, refers to an invasion of a biological system, typically of an organism, by one or more agents (or pathogens) that are not generally associated with the biological system. The agent is often a disease-causing agent. Infection also includes the propagation and multiplication of the agent, and the reaction of host biological system or organism. Infection also includes the generation of toxins, by or as a proximal cause of, the agent. Infectious disease, sometime referred to as “transmissible disease” or “communicable disease,” is a disease state resulting from an infection. Pathogens include, but are not limited to, viruses and related agents such as viroids and prions, bacteria, fungi which may be further classified, for example, as Ascomycota, including yeasts such as Candida, filamentous fungi such as Aspergillus, Pneumocystis species, and dermatophytes, Basidiomycota, including the human-pathogenic genus Cryptococcus, parasites which may be further classified, for example, as unicellular organisms (including, for example, malaria, Toxoplasma, Babesia), Macroparasites (including worms or helminths) such as nematodes such as parasitic roundworms and pinworms, tapeworms (cestodes), and flukes (trematodes, such as schistosomiasis), arthropods such as ticks, mites, fleas, and lice, can also cause human disease, which conceptually are similar to infections. Invasion of an animal body, such as a human body, by macroparasites may also be termed infestation but is consider, as used herein, to be a form of infection.

“Inflammation,” as used herein, refers to a particular, generic set of biological responses of body tissues to stimuli, such as pathogens, damaged cells, or irritants. Inflammation (and the associated condition, pre-inflammation) is a response involving immune cells, blood vessels, and molecular mediators that, at least in part, serves to eliminate the initial cause of cell injury, clear out necrotic tissues damaged from the original insult and initiate tissue repair. Signs of inflammation include increased heat, pain, redness, swelling, and loss of function. Inflammation may be considered a mechanism of innate immunity, as compared to adaptive immunity, which would be specific to a particular pathogen. Inflammation may be classified as acute or chronic. Acute inflammation is the initial response of the body to a stimuli and may be achieved by the increased movement of plasma and leukocytes (especially granulocytes) from the blood into the injured tissues. A series of biochemical events propagates and matures the inflammatory response, involving the local vascular system, the immune system, and various cells within the injured tissue. Chronic inflammation, often termed prolonged inflammation, may cause a progressive shift in the type of cells present at the site of inflammation, such as mononuclear cells, and is characterized by simultaneous destruction and healing of tissue.

“Metabolic task”, as used herein, refers to an event or process in an organism which consumes stored energy to perform physical, chemical, or electro-chemical work-maintenance and repair of structure, disposal of waste, execution of function-typically accompanied by a release of heat from inefficiencies in the energy transformation.

“Metabolic ecology,” as used herein, refers to one or more of the organizing paradigms of biology, based on energy expenditure, energy budget, and health capacity. Metabolic ecology defines inter- and intra-species variations and interactions related to resource dependencies and allocations. Metabolic ecology may be considered as a sub-field of ecology aiming to understand constraints on metabolic organization as important for understanding almost all life processes, where the main focus is on the metabolism of individuals, emerging intra- and inter-specific patterns, and the evolutionary perspective. Models of individual's metabolism follow the energy uptake and allocation, and can focus on mechanisms and constraints of energy transport (transport models), or on dynamic use of stored metabolites (energy budget models). Two main metabolic theories, upon which the present technology may be justified but is not necessarily premised, and upon which it does not necessarily rely, may underpin an individual-based metabolic ecology understanding are Kooijman's Dynamic energy budget (DEB) theory and the West, Brown, and Enquist (WBE) theory of ecology.

“Metabolism,” as used herein, refers to transformation of energy by converting chemicals and energy into cellular components (anabolism) and decomposing organic matter (catabolismiving things require energy to maintain internal organization (homeostasis) and to produce the other phenomena associated with life.

“Micro-physiometry”, as used herein, refers to in vitro measurement(s) of functions and activities of life or of a biological system (such as organs, tissues, or cells) and of the physical and chemical phenomena involved on small (at or sub-micrometer) scale. The primary parameters assessed in micro-physiometry comprise pH and the concentration of dissolved oxygen, glucose, and lactic acid, with an emphasis on the first two. Measuring such parameters experimentally, for example in combination with a fluidic system for cell culture maintenance, and a defined application of drugs or toxins provides the quantitative output parameters extracellular acidification rates, oxygen consumption rates, and rates of glucose consumption or lactate release to characterize the metabolic situation.

“Oncogenesis,” as used herein, refers to the formation of a cancer, whereby normal cells are transformed into cancer cells, also termed “tumorigenesis” or “carcinogenesis”. The process is characterized by changes at the cellular, genetic, and epigenetic levels and abnormal cell division. Mutations in DNA and epimutations disrupt processes involved in the programming and regulation of the normal balance between proliferation and programmed cell death.

“Organization,” as used herein refers to the state of being structurally composed of one or more cells—the basic units of life.

“Osmometer,” as used herein, refers to a system, device, or process for measuring the osmotic strength of a solution, colloid, or compound. Osmometers may be used in determining the total concentration of dissolved salts and sugars in blood or urine samples, or in determining the molecular weight of unknown compounds and polymers. For example, vapor pressure osmometers determine the concentration of osmotically active particles that reduce the vapor pressure of a solution; membrane osmometers measure the osmotic pressure of a solution separated from pure solvent by a semipermeable membrane; and freezing point depression osmometers may determine the osmotic strength of a solution, as osmotically active compounds depress the freezing point of a solution.

“Pre-inflammation,” as used herein, refers to a biological stage that proceeds the clinical definition of “inflammation” as measured by canonical detection methods.

“Processing,” as used herein, refers to the collection and manipulation of items of data to produce meaningful information or change of information in any manner detectable by an observer. Processing of data may include, sorting, summarization, aggregation, analysis (collection, organization, interpretation and presentation), reporting, or classification of data.

“Processing system,” as used herein, refers to a system (be it electrical, mechanical or biological) which takes information (a sequence of enumerated symbols or states) in one form and processes (transforms) it into another form, e.g. to statistics, by an algorithmic process. A processing system generally includes input, processor, storage, and output.

“Reproduction,” as used herein, refers to the ability to produce new individual organisms, either asexually from a single parent organism or sexually from two parent organisms.

“Response (to stimuli),” as used herein, refers to an action or modification in a biological system that results from external stimulus. A response may take any of several forms. For example, in the case of a unicellular organism, it may be the contraction resulting from exposure to the presence of chemicals in the environment. As another example, response may be a complex set of reactions involving all the senses of multicellular organisms. A response is often expressed by motion; for example, the leaves of a plant turning toward the sun (phototropism), and chemotaxis.

“Storage,” as used herein, refers to recording or storing of information or data in a storage medium, e.g. DNA and RNA, handwriting, phonographic recording, magnetic tape, optical discs, and semiconductor memory. In computers, data storage generally consists of semiconductor-based integrated circuit (IC) chips such as dynamic volatile semiconductor random-access memory (RAM), particularly dynamic random-access memory (DRAM).

“Stress,” as used herein, refers most generally to a state of a biological system being subjected to an external demand. This demand must be compensated by some energy expenditure with the consequence of a decreased health capacity. Stress is, typically, a response to one or more external stimuli. Stress may be manifest by modification or dis-regulation of the nominal, typical homeostatic operation of the biological system or of a portion of the system (for example, sub-systems, organs, tissues, etc.). Examples of stress include anxiety, disturbance of sleep patterns, pre-inflammation, inflammation, elevated heart rate, elevated blood pressure, and chronic pain.

“Thermometer,” as used herein, refers to a device that measures temperature or a temperature gradient. The device comprises a temperature sensor in which some change occurs with a change in temperature, and some means of converting this change into a numerical value. A thermometer may utilize the property of thermal expansion of various phases of matter, measure the vapor pressure of a liquid, detect density changes of a liquid in proportion to its temperature, utilize thermochromism (i.e., the property of some substances to change color due to a change in temperature), utilize temperature-dependence of the band gap of semiconductor materials (i.e., band edge thermometry), detect black body radiation (e.g., pyrometer, infrared thermometer, and thermography), exploit luminescence emitted by phosphor material which changes with temperature, or utilize the temperature dependence of optical absorbance spectra, electrical resistance, Seebeck effect, nuclear magnetic resonance, or magnetic susceptibility of a material.

“Thermometry,” as used herein, refers to systems for or process of measuring of a current local temperature, which is a physical property of matter that quantitatively expresses the relative presence or absence of thermal energy. When a biological system is in the state of local thermodynamic equilibrium (i.e., no macroscopic chemical reactions or flows of matter or energy), the temperature of the system is related to the average kinetic energy of the molecules in the system. Many real-world systems are not in thermodynamic equilibrium and are not homogeneous, however, local thermodynamic equilibrium can be assumed for appropriate spatial and temporal scales.

“Viral infection,” as used herein, refers to infection of a biological system by a virus. Viral infections include, for example (i) respiratory infections, which are viral infections of the nose, throat, upper airways, and lungs, such as the common cold, influenza, pneumonia, infection by SARS-COV-2, which causes the disease condition known as Coronavirus Disease 2019 or COVID-19, croup (inflammation of the upper and lower airways, often referred to as laryngotracheobronchitis) or lower airways (often referred to as bronchiolitis); (ii) gastrointestinal tract infections, which are viral infections of the gastrointestinal tract, such as gastroenteritis, are commonly caused by viruses, such as noroviruses and rotaviruses; (iii) liver infections, which may result in hepatitis; (iv) nervous system infections, such as the rabies virus and the West Nile virus, which infect the brain, and may cause encephalitis; (v) infection of tissues that cover the brain and spinal cord, such as meninges, which may cause meningitis or polio; (vi) skin infections, which may affect not only the skin but also sub-dermal tissue, and may result in warts, rashes, or other blemishes, such as chicken pox or shingles; (vii) placenta and fetal infections, such as the Zika virus, the rubella virus, and cytomegalovirus, which may infect the placenta and fetus in pregnant women; and (viii) viruses affecting various systems, including for example enteroviruses (such as coxsackieviruses and echoviruses) and cytomegaloviruses.

“Water*” as used herein, refers in its most broad aspects to systems comprising or containing water, including aqueous systems. More specifically, it refers to systems in which water serves as a solvent or medium for one or more solubilized components, such as ions, or suspended components, such as lipids. The amount of water in an aqueous system may be as low as 5% or 10% by weigh, or a high as 70%, 80%, 90%, 95%, 99%, greater than 99% by weight. Water* includes species associated with water in such systems, including but not limited to other water molecules, ionic or radical forms of oxygen or hydrogen and oxygen, and combinations of these with carbon or non-carbon substances, for example, elements, ions, molecules, cofactors, minerals, salts, polymorphs or mixtures. A system is defined as aqueous not by the fraction of water present but by the role played by water in the system.

### General Criteria for Identifying and Selecting Emergent Factors

Emergent factors or emergent properties are identified and selected, for measurement, based on various criteria. Generally speaking, emergent factors that may be directly measured are preferred over emergent factors that may only be measured indirectly. Techniques used to measure (directly or indirectly) that are less invasive are preferred over techniques that are more invasive. Measurements that are reliable and associated with a single emergent factor, rather than multiple emergent factors, are preferred.

### General Criteria for Identifying and Selecting Data to Be Measured

Table 1 lists various examples of physical properties of water*; these properties may be monitored, as described, and relate to emergent properties of biological systems. For each identified property, the following are described: inherent chemistry of the property, relevant proto-biochemistry of the property, relevant Enzyme Commission number (EC #) relating to the property, and examples of direct and/or indirect measurements from which data relating to the property may be derived. Data streams of or relating to these measurements may be fed into a learning engine to develop arrays of measurement. Such arrays of measurements permit an understanding of the functions of a biological system, such as a protocell or an organism, and permit quantification of health capacity of the biological system.

A learned model of the learning engine may be used to predict and/or optimize the functions of the biological system, such as a protocell or an organism, and to modify and or design and engineer the biological system, such as a protocell or an organism, and to develop an understanding of the “rules of health capacity” and how homeostasis is controlled.

The Enzyme Commission number (EC #) described may represent a class of biochemical reactions an enzyme catalyzes. For example, EC1 stands for oxidoreductase, EC2 stands for transferase, EC 3 stands for hydrolase; EC4 stands for lyase, EC5 stands for isomerase, EC6 stands for ligase, and EC7 stands for translocase.

TABLE 1

| | | | | Direct | |
|---|---|---|---|---|---|
| | Inherent | Proto- | | Physical | Indirect |
| Property | Chemistry | Biochemistry | EC# | Measurement | Measurement |
| Amphoteric | Ionization | Readily generates | EC3 | pH | |
| | | protons | | | |
| Conductivity | Solubilization | Readily solvates | EC7 | Amperometric | Sensor Beta |
| | | ions | | | |
| Solvation | Solubilization | Solvates ions to | — | Spectroscopic | |
| Capacity | | high concentration | | | |
| Ion Mobility | Solubilization | Readily allows ion | EC7 | Bio-impedance | Sensor Beta |
| | | motion | | | |
| Oxidation- | Redox | Readily generates | EC1 | Potentiometric | |
| Reduction | | electrons | | | |
| Ligand | Coordinate | Readily generates | EC1- | Spectroscopic | |
| | Covalent | metal complexes | EC7 | | |
| Hydration | Hydrolysis | Readily hydrates | EC3 | Spectroscopic | |
| | | and dehydrates | | | |
| Electrolysis | Redox | Catalytically | EC7 | Spectroscopic | |
| | | generates oxygen | | | |
| Thermal | Heat transfer | Readily transfers | EC1- | Calorimetric | Sensor Alpha |
| Conductivity | | heat | EC7 | | |
| Heat Capacity | Thermal | Readily withstands | — | Thermometry | Sensor Alpha |
| | reservoir | environmental | | | |
| | | temperature change | | | |
| Thermal | Waste heat | Readily scavenges | — | Spectroscopic | |
| Absorptivity | Retention | waste heat of | | | |
| | | exothermic | | | |
| | | reactions into | | | |
| | | thermal reservoir | | | |
| Adhesion | Chemotaxis | Readily enables | EC1- | TBD | |
| | | solute diffusion | EC7 | | |
| Cohesion | Structure | Readily enables | — | Doppler | |
| | | flow | | | |
| Cohesion | Structure | Readily enables | — | Multiple | |
| | | communication | | | |
| Cohesion | Structure | Readily enables | — | Microscopy | Sensor Gamma |
| | | persistent form(s) | | | |
| Adhesion | Structure | Readily enables | — | Microscopy | |
| | | structural | | | |
| | | boundaries | | | |
| Transparency | Structure | Readily allows | — | Spectroscopy | |
| | | energy/information | | | |
| | | transfer | | | |
| Incompressibility | Structure | Inherent structural | — | Ultrasonic | |
| | | rigidity | | | |
| Dipolarity | Electrical | Inherent shielding | — | Multiple | |
| | | against strong | | | |
| | | electric fields | | | |
| Diamagnetism | Magnetic | Inherent shielding | — | Various | |
| | | against string | | | |
| | | magnetic fields | | | |
| Wide voltage | Redox | Allows compounds | — | Voltammetric | |
| range of liquid | complexity | with large diversity | | | |
| phase | | of redox potentials | | | |
| | | to be co-dissolved | | | |
| Wide | Liquidity | Allows biosphere to | — | Rheologic | Sensor Gamma |
| temperature | | extend over | | | |
| range of liquid | | majority of the | | | |
| phase | | biosphere | | | |
| Abundancy | Stability | Readily allows | — | Volumetric | |
| | | biology to scale | | | |
| Speciation | Redox | Readily allows | EC1 | Spectroscopy | |
| | | cross coupling of | | | |
| | | chemical reactions, | | | |
| | | e.g., photosynthesis | | | |
| | | with respiration | | | |

### General Criteria for Selecting and Designing Sensors

The criteria for designing and selecting sensors, and for selecting measurements to be made by such senosrs, are based on detection and quantification of heat and work over time to enable the construction of an energy signature. An energy signature when annotated with metabolic tasks gives us an energy budget and the energy budget allow us to quantity health capacity, maximize health, intercept disease, learn the Health Capacity Rules and the rules of homeostasis.

The First Law of Thermodynamics states that energy is neither created nor destroyed but that it may change forms in diverse ways. This law does not weaken as systems become more complicated. We observe that the conservation of energy as it travels through a biological system presents an opportunity to quantify energy expenditures which are typically thought to be inaccessible, for example the energy of metabolic tasks, chemical work done inside a cell. These metabolic tasks consume energy in two ways. First is the production of heat. Heat released by metabolic activity leaves the body quickly (within less than a minute). Second is the performance of work. Work often results in energy being stored in various physical or chemical forms that can be released later as heat and/or further work.

An analytic insight of the disclosed technology is that heat is much easier to quantify than work because it can be measured externally and also that heat includes energy previously expended as work. Therefore, non-invasive measurement of heat or changes in heat flow enables quantification of the cellular work assuming the heat can be accurately mapped back to an earlier work expenditure. To this end we will quantify the work of various metabolic tasks by associating timestamped annotations of those tasks with the energy signature record of heat. Additionally, auxiliary sensors (e.g. an accelerometer) and mobile applications will collect data and metadata to characterize the metabolic tasks in terms of various attributes, such as: size, duration, intensity, quality, type. We can then learn to associate detailed archetypal heat signatures with each metabolic task, sensitive to detailed characterizations, using AI/ML to create a predictive model of the energy signature. This model will afford the user real time quantification of work associated with metabolic tasks and an understanding of implications for their energy budget.

While there is no single optimal way to characterize metabolic tasks there is an optimal way to characterize the dominate forms of heat and work in a biological system. This, in addition to feasibility concerns, informs our choice of sensor in initial prototypes.

Future versions will be determined by the empirical analysis of energy conservation. We will discover periods where our inferred budget appears to have gaps. We will correlate budget gaps with activities, metabolic tasks, or atmospheric conditions. We will deliver new passive sensors to detect and characterize the missing heat or work with more accuracy. By using the energy budget as a guide in this manner we will identify and quantify all energy consuming processes in biology which necessarily will result in a quantification of health capacity.

### Examples of Sensors and Measured Data, and Use to Quantify Aspects of Adaptive Capacity Sensor Example No. 1: Temperature and Heat Flux

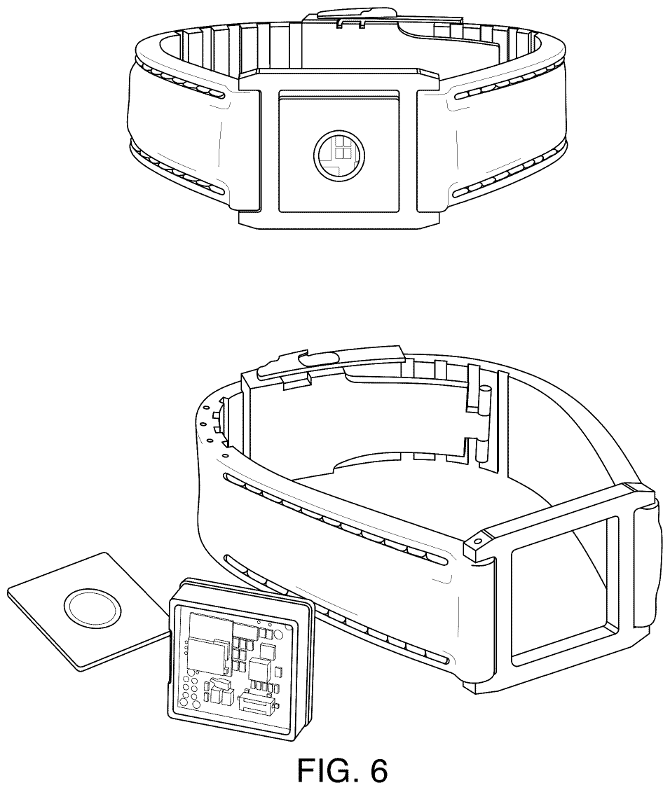

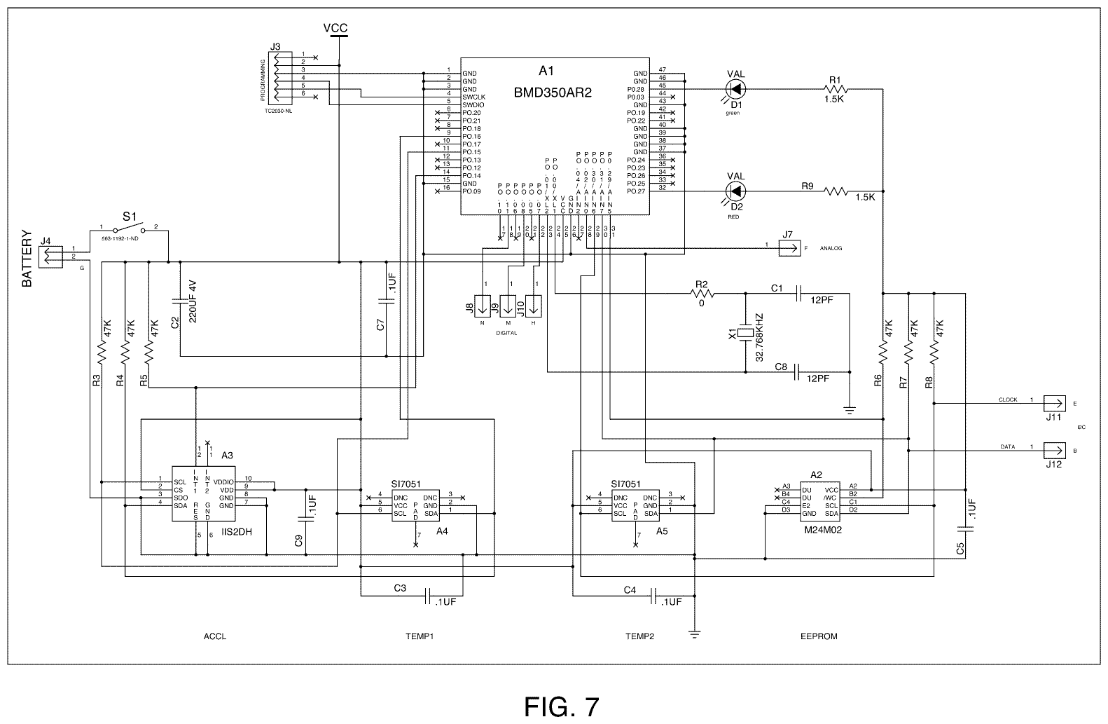

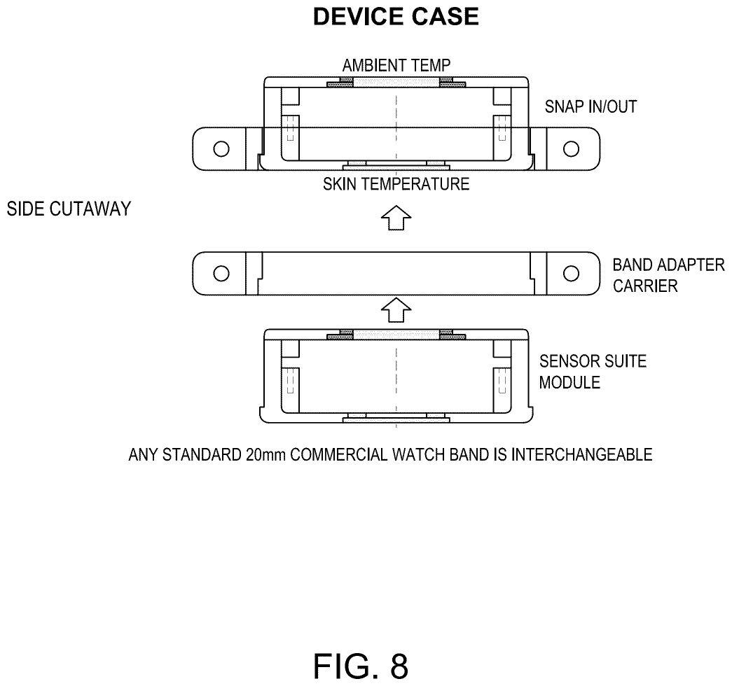

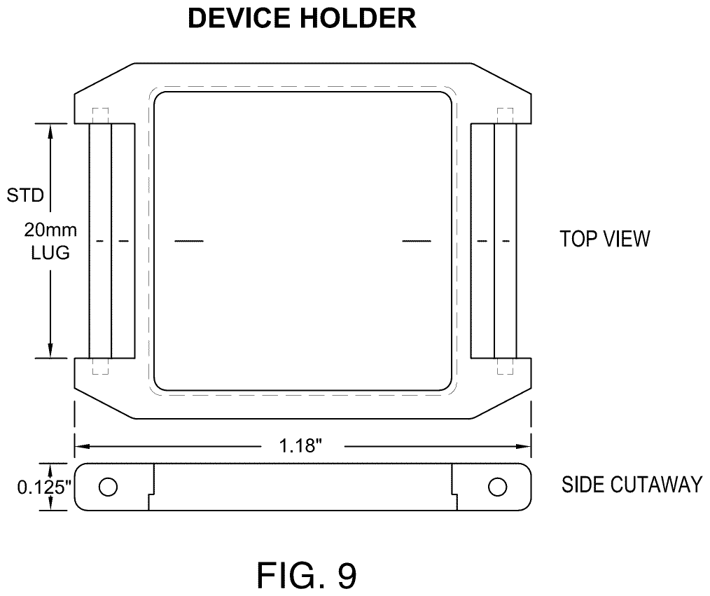

In one embodiment of Sensor Example No. 1, the system described herein includes a sensor comprising a sensor module to measure skin temperature and ambient temperature. For example, as shown in FIGS. 6, 7, and 8, a sensor device may comprise a sensor module or modules to measure skin temperature and ambient temperature and generate data reflecting such measurements. Information about the temperature or core temperature of a biological system may be inferred from such measurements. In others embodiment, as shown in FIGS. 6, 7, 8 and 9, the system may comprise a sensor module to measure simultaneously, or substantially simultaneously, at least the following properties: (i) skin temperature, preferably to the precision of 0.1° C., (ii) ambient temperature, and (iii) motion in three dimensions. The system and sensors are preferably designed to measure transducer inputs, of analog or digital form, and generate related data. The system and sensors may be wearable by an organism, such as by a person, and may be mounted to the arm, chest, leg, abdomen, or anywhere on the body. The device may include a memory element which stores measurement data for more than a month.

In another example, as shown in FIGS. 6, 7, 8, 9, and 10, the device may comprise a wireless transmission module to transmit data to be displayed on a smartphone or a tablet. The device may comprise a battery which can last for about six months when operating at a condition that transmits wireless signals at 30-second intervals. The device may comprise a gas gauge indicator for battery life. The device may transmit information regarding battery life to be displayed on a smartphone or a tablet. The device may be controlled through the smartphone or tablet. For example, the signal transmission intervals may be adjusted. The device may comprise a LED which indicates the status of communication or operation, or alerts and error. The device may be water resistant.

In another embodiment of Sensor Example No. 1, the system described herein includes a sensor comprising a sensor module to measure skin temperature and ambient temperature to enable the determination of heat flow or flux. Today, the current gold-standard for determination of heat flow is calorimetry. Direct calorimetry is a reliable standard for measuring of heat flow. In biology, direct calorimetry is employed to quantify the flow of heat generated as a byproduct of metabolism (exothermicity). Direct calorimetry on humans is highly impractical as it requires a subject to be isolated in a “room calorimeter.”

Indirect calorimetry overcomes the inconvenience of direct calorimetry by using surrogate measures to quantify heat flow. One means of indirect respiratory calorimetry is the measurement of oxygen consumption and carbon dioxide product. This method is considered a clinical gold standard. Indirect calorimetry is based on the assumption that the majority of heat generation is derived by oxidative phosphorylation-carbon oxidation (CO2 production) and oxygen reduction (oxygen consumption). Indirect clinical calorimetry requires expensive centralized testing on individual subjects.

An example of a calorimetry sensor of the disclosed technology, termed Sensor Example No. 1 for convenience, employs approximate direct calorimetry to measure heat flow directly in biological systems, for example organism, for example humans. In some embodiments, Sensor Example No. 1 is a miniaturized calorimetry sensor. In some embodiments, Sensor Example No. 1 contains at least two pre-calibrated solid-state digital temperature sensors that simultaneously measure skin temperature and ambient temperature of the air proximate to the point of skin temperature measurement. By continuously measuring the local difference between ambient and skin temperature, Sensor Example No. 1 quantifies heat flow (loss) through the skin—the predominant mode of energy loss in humans.

Sensor Example No. 1, in one embodiment, is neither as accurate nor as precise as direct or indirect clinical calorimetry, but Sensor Example No. 1 has at least the following advantage over such methods: the continuous measurement of metabolic rate at scale. This enables Sensor Example No. 1, in certain embodiments, to detect even subtle changes in metabolic rate over long period of time-days—and to employ automated machine learning on both individuals and populations. See, for example, Table 2, below.

There exist differences between thermometry, thermography and calorimetry even though these three concepts are often conflated. Thermometry and thermography—the use of a thermometer or thermal imager—are devices and processes that measure the relative temperature of a body of matter. Calorimetry is a system or process that measure absolute heat flow from a body of matter. Thermometers and thermal imagers are used as surrogates of core temperature to measure fever, not heat flow. In contrast, Sensor Example No. 1, in certain embodiments, is configured to use solid state temperature sensors not to measure fever, but to measure heat flux. All of the current clinically available skin “thermometers” measure skin temperature as a proxy for core body temperature-fever—not heat flux. As they do not quantify ambient temperature, and therefore cannot calculate radiant heat transfer, they cannot detect (nor claim they can) precise or accurate changes in heat flow and metabolic rate that are obtained with Sensor Example No. 1.

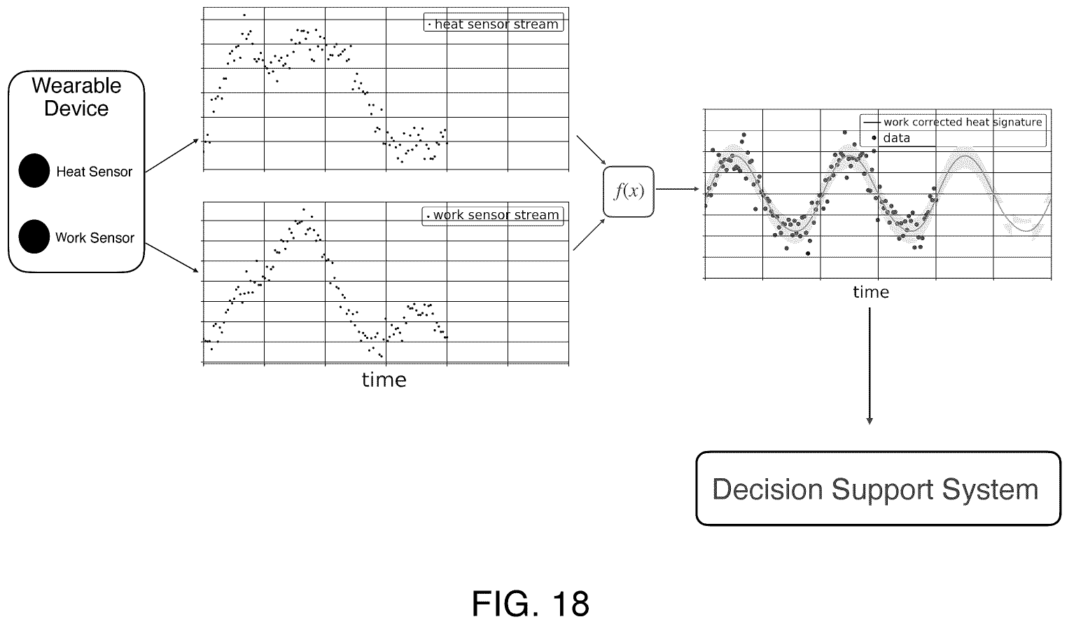

In one specific embodiment of Sensor Example No. 1 shown in FIG. 18, the system comprises a combination of work sensors and heat sensors of any kind. The system may be implemented as a wearable device for the real time evaluation of a basal or resting metabolic status.

Thermoregulation consists of two principal components: heat production and heat elimination. Healthy homeostasis requires a balance between the two. There is a dynamic balance in which the two components remain roughly equal through a large range of energy demands and rate of change. By measuring the two components continuously and analyzing their temporal alignment, the system can assess health in a much more detailed manner than if only one or the other were measured.

In the embodiment shown in FIG. 18, the heat sensor may comprise an array of thermometers, and heat elimination can be estimated as a differential temperature at the biological system′ skin surface. Differential temperature at the skin surface does not quantitatively equal heat point by point, but has a correlation with real time heat elimination. To obtain an estimate of heat production, an accelerometer can be used as the work sensor to detect physical activity and infer the work done by the biological system.

FIG. 18 shows the temporal alignment between peaks in the work sensor signal stream (e.g., accelerometer measurement over time) and the heat sensor signal stream (e.g., differential temperature over time). During physical exercise, the heat elimination signal may be interpreted as having two components, a resting component and an activity component. In other words, the Total Energy Expenditure can be approximated by the summation of Resting Energy Expenditure and Physical Activity Energy Expenditure. Therefore, the work sensor signal stream may be subtracted from the heat sensor signal stream to obtain the work-corrected heat signature data stream, and the work-corrected heat signature data stream may be fed into a decision support system to infer the basal or resting metabolic rate.

In some embodiments, the decision support system may process the signals as a function of time by generating a first vector, said first vector comprising features extracted from said signals, wherein said features comprise: mean Peak (P) amplitude, Peak (P) mean amplitude, Peak (P) std of amplitude, Through (T) amplitude, Trough (T) mean amplitude, Trough (T) std of amplitude, peak to peak intervals, Peak-to-Peak High Frequency (P-P HF) Power, and any combination thereof; transforming said first vector into a second vector, said transformation comprising normalization; and applying a classification algorithm adapted to classify said second vector, wherein said classification algorithm comprises an ensemble of classification and regression trees.

In some embodiments, the decision support system may use a machine learning classifier such as a support vector machine (SVM) classifier, a Naïve Bayes classifier (NBC), and an artificial neural network (ANN) classifier to analyze the signals. In some embodiments, the decision support system may use a Hidden Markov Model (HMM) algorithmic process that considers temporal dynamics of the signals. In some embodiments, the decision support system extracts a plurality of features from the signals by generating a feature vector for each time period. In some embodiments, the decision support system extracts a plurality of features from the plurality of signals by dividing each of the plurality of signals into overlapping time period windows that are shifted. The shifting of overlapping time period windows results in a plurality of epochs that are analyzed. For each epoch of the plurality of epochs, a plurality of features (e.g. mean, standard deviation, frequency-domain features, entropy, etc.) are calculated that characterize different relevant aspects of the plurality of signals. A feature vector is generated for each epoch of the plurality of epochs and the feature vector consists of the plurality of features. The feature vector is inputted into the machine learning classifier to automatically classify each epoch.

Independent component analysis (ICA) and/or principal component analysis (PCA) can be applied as well to find any hidden signals. Temporal features may then be computed from this (potentially improved) signal representation. For temporal features, various nonparametric filtering schemes, low-pass filtering, band-pass filtering, high-pass filtering, may be applied to enhance desired signal characteristics.

Parametric models such as AR, moving average (MA), or ARMA (autoregressive and moving average) models may be used, and the parameters of such a model may be found via autocorrelation and/or partial autocorrelation, or LPA, LMS, RLS, or Kalman filter. The entire or part of estimated coefficients may be used as features.

In some embodiments, the decision support system may adapt or utilize three levels of heat signature metrics, using, for example, machine readable instructions to record, process, display, or otherwise implement these metrics:
Level 0 Metrics: A heat signature may be usefully quantified in terms of very basic metrics; for example mean, variance, minima and/or maxima, etc. These metrics/statistics may be used, directly, as indicators of health status or change thereof.
Level 1 Metrics: More sophisticated metrics of heat signature incorporate one or more principles or concepts of biological organization and circadian health. For example, a principle may include, from the circadian rhythm literature, concepts of Inter-day Stability (IS) and/or Intraday Variability (IV), non-parametric statistics of circadian structure. An alternative parametric approach to circadian analysis involves Cosinor fitting. This can be applied to any signal which has a periodic component. This category of metrics may be complicated, involving the fitting of many parameters, each of which is potentially an independent metric of health capacity which are more informative than the simple level 0 statistics. This level may also include automated learning such as Artificial Intelligence (AI)/Machine Learning (ML).
Level 2 Metrics: Although the Level 1 metrics may be highly informative, they are not necessarily interpretable or transferrable between individuals. Level 2 metrics address these limitations by comparing a metric to its historical baseline for a single individual. By engineering a wearable device to continuously collect a heat signature over a period of weeks or months, we can construct detailed baselines. These baselines enable one to attach high degrees of confidence/significance to changes in metrics of a heat signature.

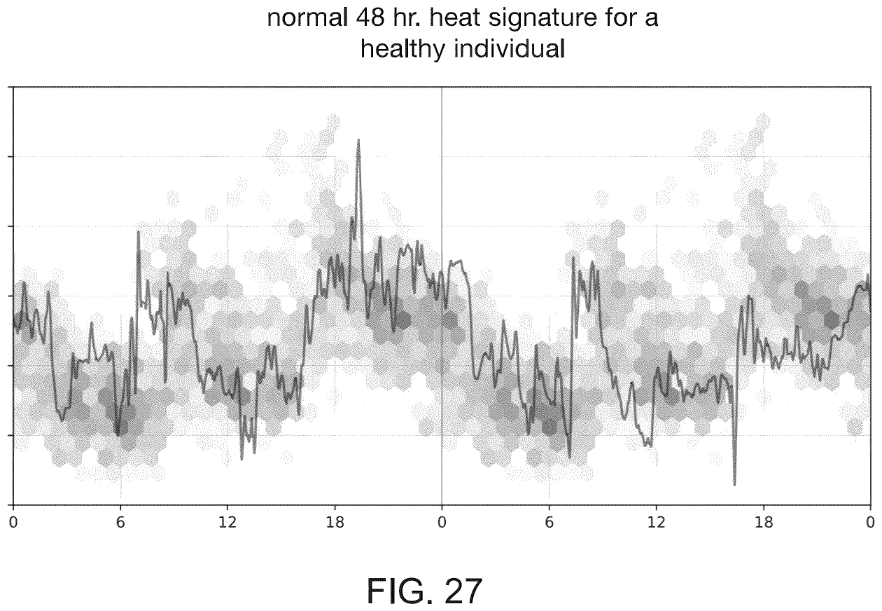

As an example, FIG. 27 shows the time series data of heat and work for a healthy person. FIG. 27 depicts a 48-hour period of a heat signature exhibiting stochastic and pseudo-periodic features, including two full sleep/wake cycles. The gray hexagonal background is a histogram which indicates values of the heat signature collected for the same individual over the preceding month. By comparing the heat signature (curve) to the gray background, one can identify extending periods of high or low heat relative to the baseline. This visual juxtaposition enables a user or medical professional to see “Level 2”-like information. There is a predictable 24-hour variation in heat elimination that coincides with sleep-wake cycles and activity.

In some embodiments, analytics software may compute, utilizing machine readable instructions, percentiles scores for relevant health metrics against the historical baseline for the metrics. In a clinical or consumer setting, alerts may be set up at percentiles thresholds (e.g., the 95th percentile) depending on level or caution or concern enabling interception of serious events or rapid correction of sub-optimal health. User interfaces which further juxtapose metrics and Level 2 contextualization of metrics along with this type of heat signature display, will enable users to develop insight and intuition about critical features of their heat signature. This feedback is critical because the stochastic nature of the heat signature is initially difficult to interpret. In some embodiments, User interfaces may incorporate the Level 2 analysis.

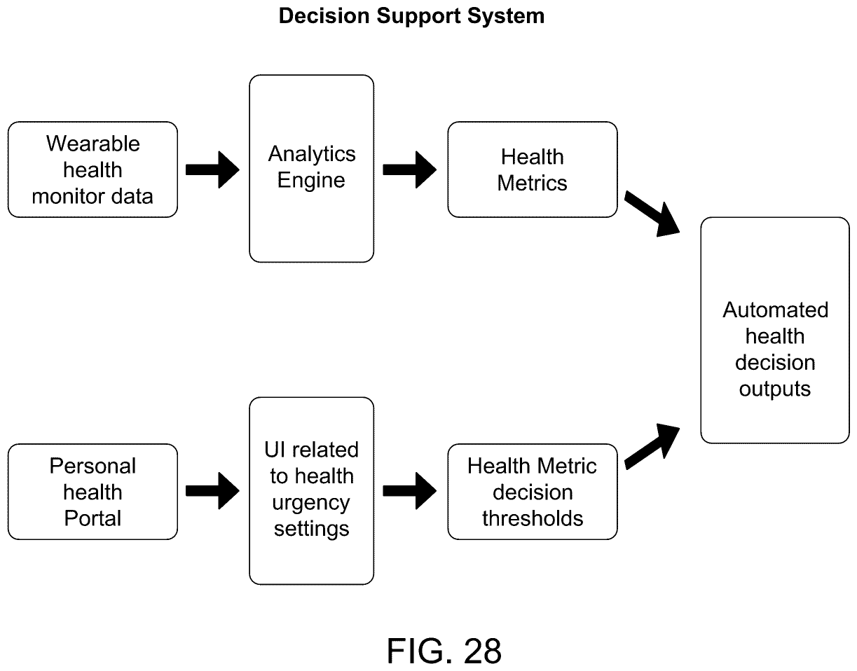

FIG. 28 illustrates an example of a decision support system which can be utilized by the embodiment of using work sensor signal to correct heat sensor signal and obtain resting metabolic status as illustrated in FIG. 18. In some embodiments, the decision support system may be based on two high level elements: 1) a data stream of information; and 2) parameters that can influence decisions made based on the data stream. The two elements may be utilized in combination when generating decision outputs.

The data stream of information is preferably relevant to an important matter of decision, and may be subject to significant uncertainty. The data stream may preferably be constructed such that it can minimize the uncertainty which is inherent to the decision-making process, similar to clinical medicine in some cases. For example, as illustrated in FIG. 28, a data stream may be “wearable health monitor data”.

In a preferred embodiment, a system for decision support involves two components associated with the aforementioned two high level elements: 1) hardware and software which stream data to a central repository and build a useful record for each individual—For example, as illustrated in FIG. 28, the “wearable health monitor data” may be fed to an “analytics engine” to extract “health metrics”; and 2) an application interface or digital portal, e.g., a “personal health portal” illustrated in FIG. 28, which allows the system to assess the acceptable range for some metric and trigger alerts when a threshold is crossed. For example, the analytics engine may use artificial intelligence, based on standard inputs or based on collected data, to identifying relevant health metrics to track, and may identify suitable and/or unsuitable ranges for each metric in the assessment of health or health capacity. As an additional example, the acceptable range may be the “health metric decision thresholds” illustrated in FIG. 28. In some embodiments, the personal health portal may be individualized. In preferred embodiments, the personal health portal may be operated by the individual, or a proxy like a family member or health professional, e.g., via the “UI (user interface) related to health urgency settings”. In the embodiment illustrated in FIG. 28., the system then combines the “health metrics” and the “health metric decision thresholds” to generate the “automated health decision outputs.” In a preferred embodiment, automated health decision outputs may include recommendations for medical and/or consumer health interventions, or suggestions for further clinical testing.

In one example relating to medical applications, a medical decision support system may preferably take inputs from two primary interfaces and obtain: 1) physiological data from a patient, and 2) parameters related to risk/sensitivity set by a doctor/patient which can determine decision thresholds to be associated with each data stream. In some cases, the primary goal of a wearable device can be to compare the data streaming from a device against the level of concern set by patients or their care provider. In some embodiments, decision support is enabled by comparing the incoming data to the distribution of historical data which has been previously acquired for the same individual.

In some embodiments, the decision support system may be implemented in a processing circuitry. The processing circuitry may comprise a correlation engine which correlates environmental factors with context data and the measured signals corresponding to the user, wherein the environmental factors, the context data are collected by one or more sensors communicatively coupled to the system, when in operation. The processing circuitry may further comprise a recommendation engine which analyzes the set of correlated data. The processing circuitry may further comprise a contextual inferencing engine which identifies, by inference, a context classification associated with sensor or perceptual readings, the readings associated with the user or the environment or behavior of the user, wherein the context classification is to be used by the correlation engine and provided with the set of correlated data. The processing circuitry may further comprise a spectral analysis unit configured to generate at least one spectral analysis signal from the measured signals. Spectral analysis may encompass using at least one of Fourier-based, wavelet based, or multifractal spectral methods. Spectral analysis may be discrete or continuous.

Analysis of the measured signals may be carried out entirely on board the wearable device, partially on board the wearable device and partially at other location(s), or entirely at other location(s). If the analysis is carried out, whether partially or fully, on board the wearable device, the wearable device also includes a microprocessor that fully or partially performs the described methods. Certain alternative embodiments can utilize a computer system other than a microprocessor to perform the methods described herein. For example, an application-specific integrated circuit (ASIC) can be used to perform some or all of the described methods.

In one implementation, the work sensors include one to three axial accelerometers. In one exemplary configuration, at least one accelerometer is configured to measure acceleration in the frontal direction, which is defined to be the direction perpendicular to the frontal plane of the user. Work sensors may include any sensing elements which allow the measurement of body motions including in particular acceleration, velocity, position, or orientation. Such may include sensors based on Micro-Electro-Mechanical Systems (MEMS) technology (e.g., piezo-resistive, or electromagnetic sensors) or optical sensors (e.g., camera based systems, laser sensors, etc.), or any other type of motion tracking sensors. Work sensors may include those measuring from single to multiple degrees of freedom, including x, y, z, pitch, roll, yaw or any combination thereof.

In some embodiments, the accelerometer can measure motions such as steps taken while walking or running, and estimate an amount of calories used. In some embodiments, the accelerometer is configured to detect an amount of energy, e.g., calories, burned by a user over a period of time. In some embodiments, the accelerometer includes power saving features. Specifically, to conserve power, the accelerometer is put into a less active, reduced power state until a certain threshold level of user activity is detected. When the threshold level of user activity is detected, a short section of the waveform is analyzed to determine whether the accelerometer signal should continue to be analyzed. The threshold for waking up the accelerometer can be a function of the history of the accelerometer signal as well as inputs from other sensors such as an ambient light sensor and skin temperature sensor. Moreover, user-specific information such as age, gender, height and weight can be used to tailor the estimate to the user.

In some embodiments, the wearable device can be worn on the wrist, belt or arm, for instance, or carried in the pocket. The wearable device can be worn during an intended workout period or as a general, all day, free living monitor, where the user may perform specific exercises at specific times while going about their daily activities at other times, e.g., including sitting, standing and sleeping. In some embodiments, the wearable device can determine what the user is doing, e.g., whether he or she is sleeping, awake, exercising and so forth and make an intelligent decision of whether the active mode should be used to gather relevant activity data from the user or to continue a power saving, reduced power mode. This monitoring can occur in the context of an all-day activity monitor.

One specific embodiment of Sensor Example No. 1 is part of a wearable device for identifying and reporting a cumulative physiological condition of an individual, comprising: at least one wearable physiological sensor for generating an electronic output in the form of a sensor output; a memory circuit containing stored mathematical algorithms for an identification of a particular cumulative physiological condition of said individual from said sensor output, said particular cumulative physiological condition selected from the group consisting of fatigue, ketosis, acute dehydration, drowsiness, edema, hypertension, shock, drowsiness, ovulation, fever, anemia and hypothermia, said mathematical algorithms for the identification of said particular cumulative physiological condition of said individual being derived from a previous sensor output compiled during a period of time that said particular cumulative physiological condition was known to have existed in said individual; a processor in electronic communication with said sensors and said memory circuit, the processor executing said stored mathematical algorithms using said sensor output to generate an output that identifies the presence of said particular cumulative physiological condition, wherein said particular cumulative physiological condition is fatigue and wherein said fatigue is identified using two functions of the stored mathematical algorithms, the two functions comprising a first function and second function for measuring total energy expenditure (TEE), the first function differing from the second function by an ability to measure a thermal effect of food (TEF), wherein the TEE comprises a sum of energy expenditures, TEE=BMR+AE+TEF+AT, wherein BMR is basal metabolic rate, an amount of energy expended by a body during rest, AE is activity energy expenditure, an amount of energy expended during physical activity, TEF is thermic effect of food, an amount of energy expended while digesting and processing food that is eaten, and AT is adaptive thermogenesis; and a display, in electronic communication with said processor which outputs said identification. The wearable device may further comprise a transceiver circuit, in electronic communication with said processor, for transmission of said presence of said particular physiological condition to said display, wherein said display is remote from said processor. The mathematical algorithms may include continuous prediction of said particular cumulative physiological condition during a period of time that said processor is receiving said sensor output signals, and/or a context detector for weighting a probability that a set of the sensor output signals illustrate a presence of said particular cumulative physiological condition for said individual.

One specific embodiment of Sensor Example No. 1 can be used to perform statistical analysis of three dimensional (3D) bodily motion data captured by motion sensors using a personal computing device. The personal exercise analysis application of the personal computing device is configured to collect 3D motion data captured from accelerometer and gyroscope, carry out statistical analysis of such data. The device is also configured to present exercise-related information and physiological information of a user on the display. The disclosed technology also incorporates analysis methods to compare exercises performed by the user to facilitate accurate and efficient learning.

One specific embodiment of Sensor Example No. 1 may be part of a system which includes a processing device and non-transitory computer-readable medium storing instructions and data. The processing device may execute the instructions to perform a series of functions. The processing device may receive sensor data including physiological data and environmental data. The processing device may further analyze historical physiological data and environmental data to determine a first correlation between a first physiological parameter and a second physiological parameter and a second correlation between an environmental parameter and the second physiological parameter. The processing device may then predict a change in a level of the second physiological parameter of an identified person for which the physiological data is received based on the first correlation and the second correlation.

Physiological parameters for living beings are typically a function of one or more of the following: the time-of-day, environmental conditions to which a being is exposed, activity level of a being, and various other physiological parameters. Some of the parameters may be related. For example, the average value of a parameter, and the variability of a parameter may change with the time-of-day based on the circadian cycle. Physiological parameters may change throughout the day on a regular schedule dependent on the time-of-day, consistent with normal circadian rhythms. Additionally, these parameters may change based on a subject's physical activity or metabolic rate. The temporal pattern of a physiological parameter may be quasi-periodic, or in certain special cases, perfectly periodic. The quasiperiodic rhythm may be of seconds-timescale, of minutes-timescale, ultradian, circadian, circalunar, or of yearly timescale.

The rhythms for a subject's temperature, heat production and heat elimination may be quasiperiodic. For example, the amplitude and frequency of change may rise and fall during the day mimicking the natural circadian rhythm. In particular, rather than a specific temperature, the subject's temperature may be maintained within an interthreshold zone. This zone may be variable between individuals and may have a circadian cycle within specific individuals. When the subject's temperature deviates below the interthreshold zone, a series of coordinated responses occur that include surface vasoconstriction, shivering, and metabolic thermogenesis, among others. When the body's core temperature deviates above the interthreshold zone, a series of coordinated responses occur that include surface vasodilatation and sweating, among others. Deviation from the interthreshold zone is also associated with behavioral patterns such as the seeking or avoidance of environmental heat.

Therefore, to evaluate a basal or resting metabolic status of the subject, the apparent measurements of a subject's temperature, heat production and heat elimination may need to be corrected for the subject's physical activity, environmental factors, as well as the subject's quasiperiodic rhythms. A number of computational methods may be used to identify the periodic components in large datasets, such as signal-to-noise based Fourier decomposition, Fisher's g-test and autocorrelation. Besides assuming a sinusoidal model, other algorithms may be used to quantify the waveform shape and the presence of multiple periodicities, which may provide vital clues in determining the underlying dynamics. For example, for the analysis of noisy datasets and other high-throughput analyses, the algorithm may incorporate a Fourier based measure that generates a de-noised waveform from multiple significant frequencies. This waveform may then be correlated with the raw oscillation data to provide oscillation statistics including waveform metrics and multi-periods.

One specific embodiment of Sensor Example No. 1 may be part of a system which analyzes the quasiperiodic rhythm of the subject, and estimates the resting state parameters of the subject based on the current state as well as the quasiperiodic rhythm. Estimate resting state parameters may be based on the relationships between resting state and current state, as well as the dynamically changing relationships between current and resting states. The system may be configured to perform transmission of the measured data and data processing.

In some embodiments, transmission of the measured data in the system may utilize modulation schemes, coding, and error code aspects. The transmission aspects include, for example, analog, digital, spread spectrum, combinatorial, and contention avoidance. The analog transmission aspects include, for example, amplitude modulation, single sideband modulation, frequency modulation, phase modulation, quadrature amplitude modulation, and space modulation methods, etc. The digital transmission aspects include on/off keying, frequency-shift keying, amplitude-shift keying, phase-shift keying, e.g., binary phase-shift keying, quadrature phase-shift keying, higher order and differential encoded, quadrature amplitude modulation, minimum shift keying, continuous phase modulation, pulse-position modulation, trellis coded modulation, and orthogonal frequency-division multiplexing. The spread spectrum transmission aspects include, for example, frequency hopping spread-spectrum and direct-sequence spread spectrum. The combinatorial transmission aspects include, for example, binary phase shift-keying with carrier frequency modulation. The contention avoidance transmission aspects include, for example, duty-cycle modulation and carrier frequency modulation. The coding aspects include, for example, wake-up schemes, preamble schemes, data packet schemes, and error code schemes. The wake-up schemes include, for example, multi-tone schemes and chirp schemes. The preamble schemes include, for example, unique identifier for packet start schemes. The data packet schemes include, for example, data related to pill type, pill expiration, manufacturer, lot number, amount, prescribing physician, pharmacy, etc. The error code schemes include, for example, repetition schemes, parity schemes, checksums, cyclic redundancy checks, hamming distance schemes, and forward error correction schemes, e.g., Reed-Solomon codes, binary Golay codes, convolutional codes, turbo codes, etc.

In some embodiments, data processing in the system may be implemented by forecasting modules that aggregate data and facilitate analysis of the aggregated data to derive predictive information. In some embodiments, population data of a plurality of subjects may be processed to derive various statistics, conclusions, forecasts, etc. Various techniques, e.g., state characterization based on multi-variate data fusion techniques, may be employed to generate various output, e.g., analyses, metrics, predictive information, etc.

In some embodiments, data processing in the system may include time-normalizing and interpolating the measured data, generating various metrics such as the average diurnal pattern, the standard deviation across days, and the overall variability, and generating predictive information. In some embodiments, data processing in the system may include assessing the regularity and stability of the circadian (diurnal) pattern of the subject.

In some embodiments, data processing in the system may include applying algorithms to one or more data sources to visualize and characterize the circadian (diurnal) pattern. Various filters or transformations may be applied to accentuate time-series features prior to metric calculation. Metrics related to variability of the daily pattern include standard deviation calculated across days, the intrinsic dimensionality calculated as number of significant principal components in the data series, the daily deviation in the average pattern or other time-series descriptive statistics.

### Sensor Example No. 2: Impedance/Electrical/Magnetic

In another embodiment, the system described herein includes a sensor comprising a sensor module to measure one or more electric properties, including for example, impedance, electric potential, and magnetic susceptibility or flux.

In some embodiments the sensor may be a bioimpedance sensor, for example, for the measurement of limb volume. The bioimpedance sensor may be selected from, for example, an electrochemical electrode, a metallic plunge probe, and a tetrapolar impedance sensor system. The bioimpedance sensor may be a tetrapolar impedance sensor system. The sensor for limb volume may be a sensor for measuring radius of curvature. The sensor for limb volume may comprise one or more circumferential strain gauges, for example, a plurality of strain gauges provided in a plurality of regions on the limb of a subject. The systems herein may further comprise multiple wireless flexible devices as described herein. For example, the system may comprise at least four wireless flexible devices, wherein a first device provides an alternating current, a second device is a ground, and additional devices are bioimpedance sensing electrodes capable of measuring differences in voltage. The first device may be placed in closer proximity to a patient's heart than said second device and additional bioimpedance sensing devices are placed between said first device and said second device. The system may further comprise four wireless flexible devices, wherein each device independently has an alternating current signal, a ground, and two bioimpedance sensing electrodes capable of measuring differences in voltage.

For example, a sensor device may comprise a sensor module or modules to measure one or more electric property, including for example, impedance, electric potential, and magnetic susceptibility or flux and generate data reflecting such measurements. Information about the electric property of a biological system may be inferred from such measurements. In another embodiment, the system may comprise a sensor module to measure simultaneously, or substantially simultaneously, at least one of the following properties: (i) impedance, (ii) electric potential, (iii) magnetic flux, or (iv) para-magnetic flux, in three dimensions. The system and sensors are preferably designed to measure transducer inputs of analog or digital form and generate related data. The system and sensors may be wearable by an organism, such as by a person, and may be mounted to the arm, chest, leg, abdomen, or anywhere on the body. The device may comprise a memory which stores measurement data for more than a month.

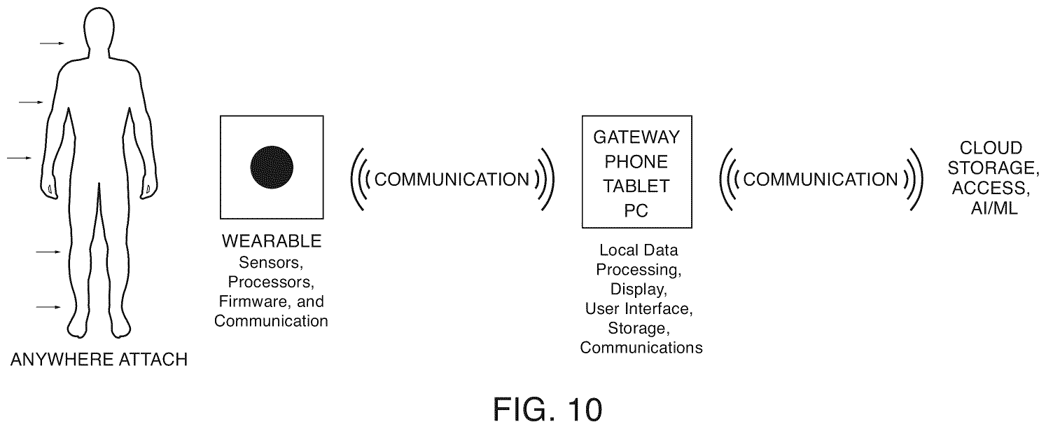

In another example, as shown in FIG. 10, the device may comprise a wireless transmission module to transmit data to be displayed on a smartphone or a tablet. The device may comprise a battery which can last for about six months when operating at a condition that transmits wireless signals at 30-second intervals. The device may comprise a charge indicator for battery life. The device may transmit information regarding battery life to be displayed on a smartphone or a tablet. The device may be controlled through the smartphone or tablet. For example, the signal transmission intervals may be adjusted. The device may comprise a LED which indicates the status of communication or operation, or alerts and error. The device may be water resistant.

### Sensor Example No. 3: Structural/Tensile/Mechanical

In one embodiment, the system described herein includes a sensor comprising a sensor module to measure tensile strength. The system and sensors are preferably designed to measure transducer inputs of analog or digital form and generate related data. The system and sensors may be wearable by an organism, such as by a person, and may be mounted to the arm, chest, leg, abdomen, or anywhere on the body. The device may comprise a memory which stores measurement data for more than a month.

### Sensor Example No. 4: Physiometry

An example of a physiometry sensor of the disclosed technology, termed Sensor Example #4 for convenience, employs at least one physiometer to measure at least one of the following emergent factors of a solution or a suspension of a biological system, such as an organism, such as a human: pH, dissolved oxygen concentration, glucose concentration, lactic acids concentration, toxin concentrations. In some embodiments Sensor Example #4 is a miniaturized physiometry sensor. In some embodiments, Sensor Example #4 contains sensors configured to measure, and degenerate data reflective of, extracellular acidification rates, oxygen consumption rates, and rates of glucose consumption or lactate release to characterize the metabolic situation. By continuously measuring these parameters Sensor Example #4 quantifies health metrics.

### Sensor Example No. 5: Redox/Electrochemical

In one embodiment, the system described herein includes a sensor comprising a sensor module to measure oxidation/reduction potential or other electrochemical properties. The system and sensors are preferably designed to measure transducer inputs of analog or digital form, and generate related data. The system and sensors may be wearable by an organism, such as by a person, and may be mounted to the arm, chest, leg, abdomen, or anywhere on the body. The device may include a memory element which stores measurement data for more than a month.

### Measurement of Health Capacity in Real-Time

There exists a need for an immediately deployable proactive health measurement system that sits alongside the current disease care system that not only is easily integrated, data based, and continuously measures health accurately, but also detects its change and indicates disease or infection pre-symptomatically. This is especially the case in rapidly progressive diseases where adaptative capacity can be depleted, narrowing a therapeutic window, and thereby worsening human and economic outcomes.

Device #1 is an example of a contemplated, commercially deployable embodiment of the disclosed system and refers to an automated wearable health accuracy measurement system and learning platform. In one embodiment, it is worn by a person, quantifying health capacity-health in real-time, enabling its optimization and the detection and interception of disease. That person could be a patient with infection obscured by immunosuppressive therapy, or a pre/asymptomatic carrier of an infectious disease, such as the SARS-COV-2 pandemic of 2019. That person could be a healthy individual seeking to maximize health or performance using sleep, nutrition, exercise, discrete neuromuscular inputs, and/or changes to lifestyle.

Device #1 may be configured to provide a rapid learning loop to enable for either health control or disease detection or interception. By employing one or more sensors (suite) Device #1 will measure and quantify emergent properties of the biological system.

In one embodiment, Device #1 senses, substantially continuously, for example at 5 second, four second, three second, 1 second, or less than 1 second intervals changes in heat or work. Device #1 is configured to automatically compute, in real time, a value that uniquely reflects function, herein termed “health capacity,” as it reflects the ability of a living system to metabolically “adapt” to persist.

While any single parameter that underpins health capacity are seemingly imprecise, collectively they are highly accurate predictors of function at a given point in time.

Device #1 is designed and configurable as a scalable automated health measurement and prediction solution. It may be an always-on, non-rechargeable requirement to enable real-time individual and population-based analytics at a global scale. To achieve this, parameters are selected that are amenable to detection (direct or indirect) by sensors with low power requirements controlled by firmware algorithms that automatically adjust sampling rate and frequency based on pre-set thresholds or changes in baseline.

Device #1 measures health changes faster enabling disease prediction and interception. Device #1 may be configured to substantially continuously measure changes in heat or work at low latency. In combination with data analytics, this allows for instantaneous calculation of homeostatic state, its change, thereby enabling the pre-symptomatic detection of disease and interception.

Changes in heat flow are sensed by two ultrasensitive solid-state monolithic CMOS IC digital temperature sensors that quantify heat flux at 1 second intervals. This allows for quantifying changes in metabolic rate.

Changes in work are sensed via osmolarity, which may be sensed potentiometrically by a 4-point contact system by quantifying impedance from 1 kHz to 1 MHz at 1 second intervals. This allows for quantifying changes in ion/flux.

Changes in work are also sensed via non-invasive measurement of cellular mechanics and/or substructure, which may be sensed at 1 second intervals. This allows for quantifying changes in tissue, cell and organelle dynamics.

Device #1 is simple, affordable, and automated. Device #1 requires no specialized skills to operate. This allows for widespread deployment of a decentralized health measurement system. Device #1 is configured to have a long a battery life, as long as for example, 100 days, 200 days, 300 days, or 400 days; it is disposable, and internally powered by a standard battery, for example, a 3-Volt (peak recharge) lithium coin cell battery of 50 mAh total capacity. This allows for continuous measurement of health trends over long period of time. Device #1 is inexpensive, lowering financial hurdles to broad deployment.

Device #1 measures parameters of health substantially continuously and is configured to interface with existing IoT architecture. This allows for early detection of change before disease symptoms and allows for the generation of big data sets to detect and learn during population-based health threats.

Device #1 may be designed to operate reliably in a harsh environment and/or harsh conditions. This allows for deployment in diverse civilian, first-responder, and warfighter settings. Device #1 may be configured to HIPPA compliant. Communication between Device #1 and standard smartphones may be accomplished over an encrypted Bluetooth Low Energy link, specifically “LESC”. No identity information about the user is stored on the wrist sensor or transmitted unencrypted by Bluetooth. Connection between the app and cloud storage servers is secure. At each stage the data is encrypted at rest (on device or cloud) and during transmission.

Secure Management of Protected Health Information: PHI will be gathered and moved to secure cloud only with consent of the Device #1 wearer. Access to data in cloud for purposes of analytics development will reveal only formally de-identified data.

Device #1 may be configured to exceed FDA requirements for a Class 2 FDA device. The sensor module is safe and may be configured for attachment to an external skin surface applied battery powered sensor suite contained within an isolated polymer (Delrin) encasement with only non-electrically conductive surface materials of exposure. There are no electrical contacts between the internal electrical circuitry and the skin.

### Platform

A wearable device enabled with FDA-approved, high precision, low-power microelectronics, capable of streaming sensor data over a secure BLE connection. Enabled by default with thermodynamic and activity sensing capabilities.

### Mobile Application

A secure software interface layer available on iOS or Android operating systems which manages cloud-based data collection, storage and analytics. Enables the conversion of raw sensor data streams to actionable health status alerts.

### Adaptable to Sensor Upgrades

Multiple new sensors configured to measure changes in heat or work, may be deployed. Components of the development of the Device #1 system include:
1. Basic principles observed and reported
2. Technology concept and/or application formulated
3. Analytical and experimental critical function and/or characteristic proof-of-concept
4. Component and/or breadboard validation in laboratory environment
5. Component and/or breadboard validation in a relevant environment
6. System/subsystem model or prototype demonstration in a relevant environment
7. System prototype demonstration in an operational environment
8. Actual system completed and “flight qualified” through test and demonstration
9. Actual system “flight proven” through successful mission operations.

### Uses of the Systems and Methods to Improve Properties of a Biological System or Non-Biological System

The systems and methods described herein are useful to improve various properties of a biological system or non-biological system. By measuring and quantifying the health capacity of a system, its efficiency and function can be improved. For example, the health capacity of microorganisms in a bioreactor could be measured and quantified, and information used to optimize or control performance. A further example would include agricultural applications, such as industrial farming or indoor farming where artificial conditions may alter the viability or sustainability of a plant or plant system. A further example would include a protocell designed and engineered to do work. In this example, the measurement and quantification of health capacity would enable more efficient work production.

### Uses of the Systems and Methods to Quantify and Control and Improve Human Health

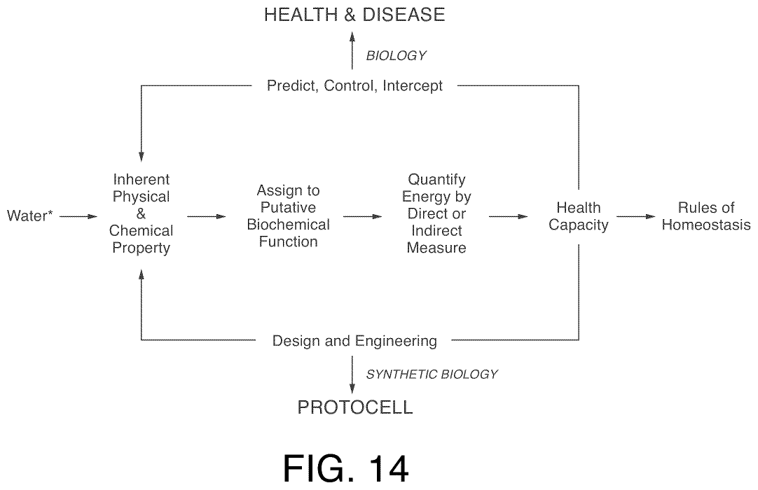

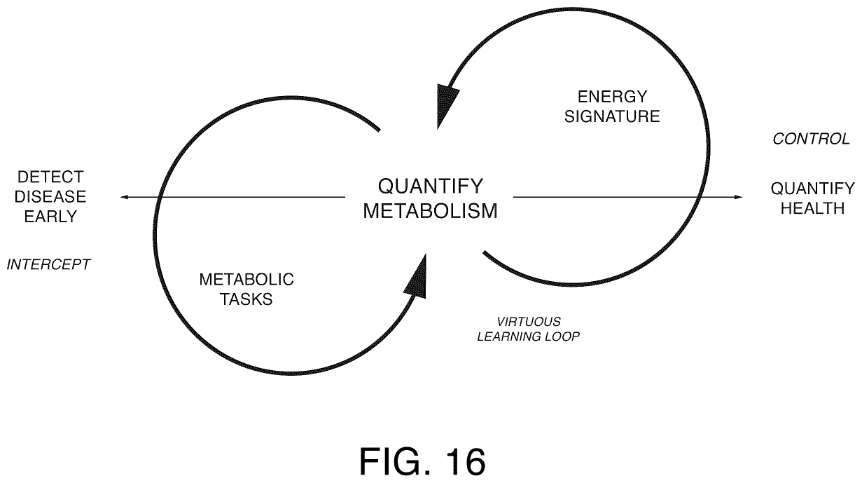

A wearable device, that is automated and measures in real-time, referred to herein as Device #1, is configured to quantify one or more emergent properties of a human subject to infer energy budgets to quantify metabolism, and thus enable control or optimization of health (as illustrated in FIGS. 11, 12, 13, 14, 15, and 16).

Currently there is no agreed upon measure of health. Health is defined as the absence of disease (symptoms). Disease metrics are lagging indicators of failing health, and thus do not reflect health, and are not in and of themselves optimizable vis-à-vis real health, not disease, outcomes. Advances in the understanding of disease have revealed that early changes in inflammation may be predictors of disease, but inflammation too is a late sign of pathogenesis.

Inflammation is a common pathway that can affect every organ system in the body. The inflammatory response can be triggered by an array of stimuli or stresses ranging from a normal response to exercise and training, to mechanisms now associated with oncogenesis and neurodegeneration. The clinical signs of inflammation have been classically defined as the pentad of: increased heat, pain, redness, swelling, and loss of function (Latin: calor, dolor, rubor, tumor, and functio laesa), and now early inflammation-so called pre-inflammation, is a risk factor for disease. We note the association between changes in water*, an emergent parameter, and inflammation, e.g., temperature and swelling.

$$Equation \#3$$

$$health \to metabolism \to homeostasis \to stress \to pre - inflammation \to inflammation \to organ specific \to systemic \to disease .$$

Device #1 measures changes in energy budgets-emergent properties that underlie health capacity—that occur prior to inflammation, pre-inflammation or disease—to enable the quantification, optimization and control of health. We note that health can be defined in a state where there is age dependent or condition loss of health capacity. Device #1 can measure a seemingly small change in health capacity that (dramatically results in disease)—so called “frailty.”

### Uses of the Systems and Methods to Detect and Intercept Disease

A wearable device, that is automated and measures in real-time, referred to herein as Device #1, is configured to quantify one or more emergent properties of a human subject to infer energy budgets—quantify metabolism—and enable early detection and interception of disease.

Currently disease is detected late. As of 2020 70% of the United States yearly healthcare budget is spent on chronic disease management. The current standard is to use disease symptoms as methods to triage illness. Disease symptoms are late indicators of disease obscuring the true underlying magnitude of injury. As a result, when they are detected significant injury has occurred, which makes understanding the true cause difficult, escalates diagnostic expense, narrows the optimal point of treatment—therapeutic window—and worsens outcomes both economic and human. These inefficiencies are generic but highlighted by the current SARS-COV-2 pandemic of 2019. Infection goes undetected, worsening individual outcome, and increased spread, and worsening containment.

Device #1 measures changes in energy budgets-emergent properties that underlie health capacity—that occur prior to inflammation, pre-inflammation or disease—to enable the quantification of loss of health, quantification of frailty, early detection and interception of disease.

TABLE 2
Translation of the pathophysiology of inflammation into physical
parameters amenable to Sensor (non-invasive) quantification.

| | Clinical | Patho- | | Surrogate | |
|---|---|---|---|---|---|
| Symptoms | Assessment | Physiology | Physics | Parameter | Sensor |
| Heat | Fever | metabolic | thermodynamic | heat flux | #1 |
| Pain | Examination | mediator | movement | acceleration | Activity, HR |
| Redness | Examination | vasodilation | thermodynamic | heat flux | #1 |
| Swelling | Examination | oncotic | electrochemical | impedance | #2 |
| | | pressure | | | |
| Loss of | Examination | mechanical | tensile/structure | particle | #3 |
| Function | | | | scatter | |

TABLE 3
Taxonomy of Use Cases for Detecting Disease States in Humans

| | | | Biological | | | |
|---|---|---|---|---|---|---|
| Use Case | Sub-system | | Response | Time | State | Named Use Cases |
| Humans | Biochemistry | Homeostasis | Inflammation | Acute | Health | e.g., reproduction, growth, |
| | → | | | | | pregnancy, performance |
| | Metabolism | | | | | (training) |
| | | | | | Disease | e.g., pathogens, trauma, toxic, |
| | | | | | | iatrogenic, (e.g., surgical, |
| | | | | | | dialysis) |
| | | | | Chronic | Health | e.g., aging, cognition |
| | | | | | Disease | e.g., x-degeneration, x- |
| | | | | | | oncogenic, metabolic, x- |
| | | | | | | fibrotic, x-genetic |

### Data and Learning Engine

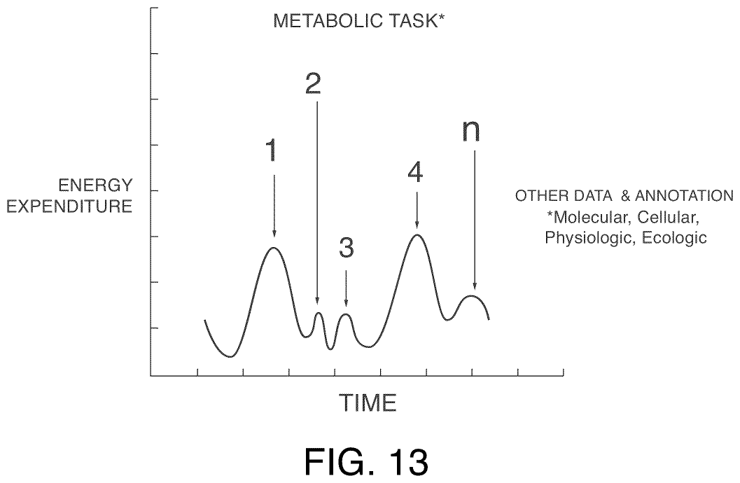

In one aspect, the disclosed technology relates to a new way to measure and learn about complex emergent adaptive systems. In some embodiments as shown in FIGS. 13, 14, 15, and 16, the disclosed methods use experiment or observation to identify emergent patterns of behavior in the system as a whole. The methods then decide what might be the most important connections or interactions between objects, individuals, or groups. The methods further construct and solve a simple model that incorporates these connections including metabolic tasks into organizing concepts that might explain the observed emergent behavior. In so doing, it is often helpful to consider organizing concepts used in models that have previously been shown to explain emergent behavior in other systems or fields. The methods further compare results and predictions with experiment or observation.

In some embodiments, the method involves developing new sensors and deriving new parameters to form a novel measurement tool. In some embodiments, the method involves using existing sensors to measure new parameters. In some embodiments, the method involves using new sensors to measure known parameters, such as annotating measurements with metabolic tasks.

In some embodiments, the method starts from first principles of physics and uses the First Law of Thermodynamics to develop the measurements and the learning engine. It is noted that non-equilibrium matter has the inherent property to self-organize, i.e., adaptive or health capacity, exemplified in “healthy” biological systems. Given that non-equilibrium thermodynamics is an unfinished domain of physics, the origin and nature of health capacity in biological systems remain mysterious. Nonetheless, the learning engine may apply general laws like the conservation of energy, as well as familiar energy expenditures like heat and work, to understand the energy budget of any non-equilibrium system. In some embodiments, by continuously measuring heat and work at scale with the sensors, the learning engine can begin to decode the thermodynamics of biological systems through a process of elimination, iteratively revealing an increasingly textured energy budget. Where inter-subject variation of this energy budget correlates with health outcomes, the learning engine can learn about health capacity. The learning engine can further learn the full spectrum of intra- and inter-subject variation of energy budget across the biosphere, which describes the health capacity rules or rules of homeostasis at various scales.

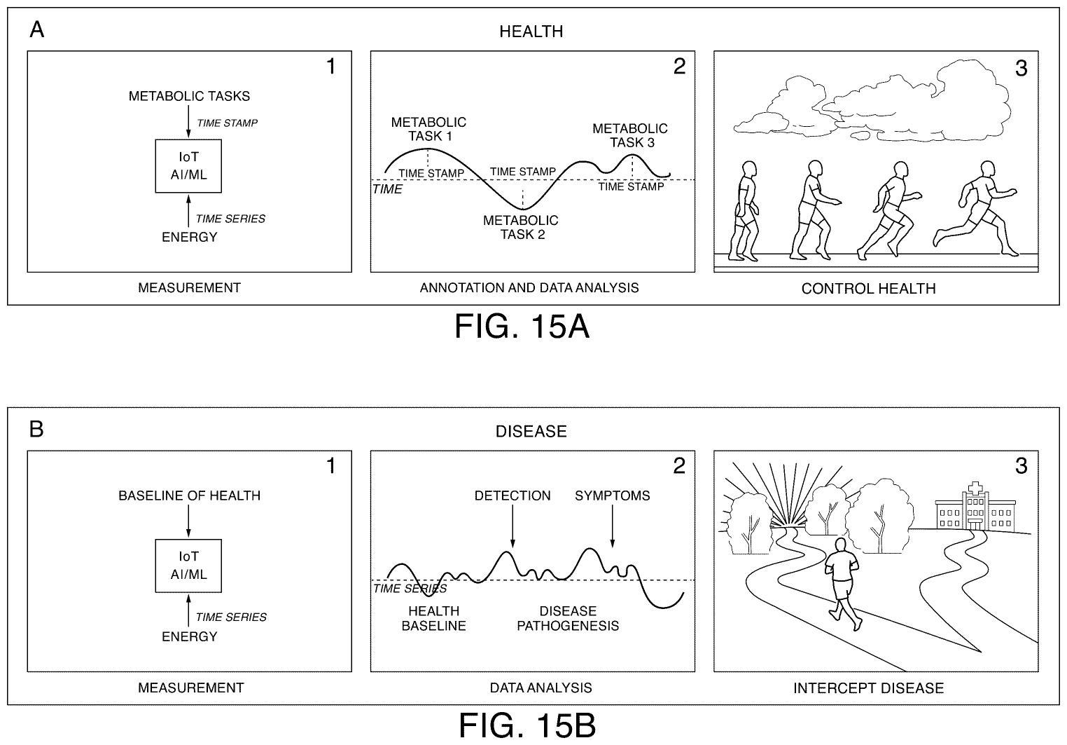

In some embodiments, data analyses (such as those depicted in FIGS. 15A and 15B, or data analysis methods performed by a learning engine) may adapt or utilize three levels of heat signature metrics:
Level 0: For instance, a heat signature may be usefully quantified in terms of very basic metrics; mean, variance, min and max, etc. These metrics/statistics may be used directly as indicators of health status or change thereof.
   Example #1: The mean of this heat signature is 2.3
   Example #2: The min of this heat signature over the last 24 hours is-0.2
Level 1: For instance, more sophisticated metrics of heat signature are built, incorporating the understanding of biological organization and circadian health. For example, the circadian rhythm metrics such as Inter-day Stability (IS) and Intraday Variability (IV), non-parametric statistics of circadian structure. An alternative parametric approach to circadian analysis involves Cosinor fitting. This can be applied to any signal which has a periodic component. This category of metrics may become quite complicated, involving the fitting of many parameters, each of which may be an independent metric of health capacity that are more informative than the simple level 0 statistics. This level may also include, for example, a human-input and/or human-training component, and/or an automated learning such as Artificial Intelligence (AI)/Machine Learning (ML).
   Example #1: The Inter-day stability of this heat signature is 0.42
   Example #2: The parameter with index #4.45.2 in the most recently trained long short-term memory (LSTM) network time series prediction has a value of −2.56
Level 2: Although the level 1 metrics may be highly informative, they are not necessarily interpretable or transferrable between individuals. Level 2 metrics address these limitations by comparing a metric to its historical baseline for a single individual. By engineering a wearable device to continuously collect a heat signature over a period of weeks or months, we can construct detailed baselines. These baselines enable one to attach high degrees of confidence/significance to changes in metrics of a heat signature.
   Example #1: The Inter-day stability of this heat signature is 0.42, or the 78th percentile with respect to typical values in the previous month
   Example #2: The rolling mean of time-resolved percentile score of the heat signature is 9.2, suggesting a severe reduction in peripheral perfusion

### Machine Learning and Predictive Modelling of Health

In one aspect, the disclosed technology relates to a new way to automate new measures and metrics of health for improving life or enabling individuals to manage/control their health efficiently, by maximizing the health capacity of a biological system or predicting and intercepting diseases in a subject, according to machine readable instructions. In some embodiments as shown in FIGS. 11, 12, 13 and 14, the new measures are based on the inherent physical and chemical properties of water*, and may be direct or indirect. A learning engine may assign a putative biochemical function to each of the measured inherent properties of water. A machine learning algorithm may quantify the health capacity and learn the health capacity rules or rules of homeostasis of the measured biological system. The machine learning algorithm may further learn, or otherwise provide insights into, how life emerged or started. Alternatively, the machine learning algorithm may extract from the measures and learn rules that govern modern biochemistry to enable discovery and development in biological and medical technologies. Further, the machine learning algorithm is configured, and designed, to extract measurements, reveal insights, and/or learn rules which allow designing and engineering of a protocell.

In some embodiments, a machine learning algorithm analyzes an energy budget based on the First Law of Thermodynamics. For example, the algorithm may calculate in real time, changes in heat (exothermicity), electrical properties (ion movement) and structure (micro-physiometry) or other forms of cellular work.

In some embodiments, the machine learning algorithm calculates the energy budget of a biological system based on the measured energy expenditure of the biological system. In some embodiments, the machine learning algorithm may learn the rules of homeostasis of the biological system.

One aspect of the disclosed technology relates to a sophisticated algorithm development process for creating a wide range of algorithms for generating information relating to a variety of variables from the data received from the plurality of physiological and/or contextual sensors. Such variables may include, without limitation, energy expenditure, including resting, active and total values, daily caloric intake, sleep states, including in bed, sleep onset, sleep interruptions, wake, and out of bed, and activity states, including exercising, sitting, traveling in a motor vehicle, and lying down, and the algorithms for generating values for such variables may be based on data from, for example, the 2-axis accelerometer, the heat flux sensor, the GSR sensor, the skin temperature sensor, the near-body ambient temperature sensor, and the heart rate sensor.

There are several types of algorithms that can be computed. For example, and without limitation, these include algorithms for predicting user characteristics, continual measurements, durative contexts, instantaneous events, and cumulative conditions. User characteristics include permanent and semi-permanent parameters of the wearer, including aspects such as weight, height, and wearer identity. An example of a continual measurement is energy expenditure, which constantly measures, for example on a minute by minute basis, the number of calories of energy expended by the wearer. Durative contexts are behaviors that last some period of time, such as sleeping, driving a car, or jogging. Instantaneous events are those that occur at a fixed or over a very short time period, such as a heart attack or falling down. Cumulative conditions are those where the person's condition can be deduced from their behavior over some previous period of time. For example, if a person hasn't slept in 36 hours and hasn't eaten in 10 hours, it is likely that they are fatigued.

The disclosed technology may be utilized in a method for doing automatic journaling of a wearer's physiological and contextual states. The system can automatically produce a journal of what activities the user was engaged in, what events occurred, how the user's physiological state changed over time, and when the user experienced or was likely to experience certain conditions. For example, the system can produce a record of when the user exercised, drove a car, slept, was in danger of heat stress, or ate, in addition to recording the user's hydration level, energy expenditure level, sleep levels, and alertness levels throughout a day.

In some embodiments, linear or non-linear mathematical models or algorithms are constructed that map the data from the plurality of sensors to a desired variable. The process consists of several steps. First, data is collected by subjects wearing a wearable device who are put into situations as close to real world situations as possible (with respect to the parameters being measured), such that the subjects are not endangered and so that the variable that the proposed algorithm is to predict can, at the same time, be reliably measured using highly accurate medical grade lab equipment. This first step provides the following two sets of data that are then used as inputs to the algorithm development process: (i) the raw data from the wearable device, and (ii) the data consisting of the gold-standard labels measured with the more accurate lab equipment. For cases in which the variable that the proposed algorithm is to predict relates to context detection, such as traveling in a motor vehicle, the gold-standard data is provided by the subjects themselves, such as through information input manually into the wearable device, a PC, or otherwise manually recorded. The collected data, i.e., both the raw data and the corresponding gold standard label data, is then organized into a database and is split into training and test sets.

Next, using the data in the training set, a mathematical model is built that relates the raw data to the corresponding gold standard labeled data. Specifically, a variety of machine learning techniques are used to generate two types of algorithms: 1) algorithms known as feature detectors that produce a result that is highly correlated with the lab-measured level (e.g. VO2 level information from a metabolic cart, douglas bag, or doubly labeled water), and 2) algorithms known as context detectors that predict various contexts (e.g., running, exercising, lying down, sleeping, driving) useful for the overall algorithm. A number of machine learning techniques may be used in this step, including artificial neural nets, decision trees, memory-based methods, boosting, attribute selection through cross-validation, and stochastic search methods such as simulated annealing and evolutionary computation. After a suitable set of feature and context detectors are found, several machine learning methods are used to cross-validate the models using the training data and increase the quality of the models of the data. Techniques used in this phase include, but are not limited to, multilinear regression, locally weighted regression, decision trees, artificial neural networks, stochastic search methods, support vector machines, and model trees.

At this stage, the models make predictions on, for example, a minute by minute basis. Inter-minute effects are next taken into account by creating an overall model that integrates the minute by minute predictions. A windowing and threshold optimization tool may be used in this step to take advantage of the temporal continuity of the data. Finally, the model's performance can be evaluated on the test set, which has not yet been used in the creation of the algorithm. Performance of the model on the test set is thus a good estimate of the algorithm's expected performance on other unseen data. Finally, the algorithm may undergo live testing on new data for further validation.

Further examples of the types of non-linear functions and/or machine learning method that may be used in the disclosed technology include the following: conditionals, case statements, logical processing, probabilistic or logical inference, neural network processing, kernel based methods, memory-based lookup (kNN, SOMs), decision lists, decision-tree prediction, support vector machine prediction, clustering, boosted methods, cascade-correlation, Boltzmann classifier, regression trees, case-based reasoning, Gaussians, Bayes nets, dynamic Bayesian networks, HMMs, Kalman filters, Gaussian processes, algorithmic predictors (e.g. learned by evolutionary computation or other program synthesis tools).

### Digital Processing Device

In some embodiments, the platforms, media, methods and applications described herein include a digital processing device, a processor, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises the Magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, user, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

While preferred embodiments of the disclosed technology have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Web Browser Plug-in

In some embodiments, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla Firefox® for mobile, Microsoft® Internet Explorer® Mobile, Amazon® Kindle® Basic Web, Nokia® Browser, Opera Software® Opera® Mobile, and Sony® PSP™ browser.

### Sensors Integration/Signal Processing

The disclosed system may use data from two or more sensors to calculate the corresponding physiological or environmental data (for example, data from two or more sensors which are used in combination).

In one embodiment, the disclosed system also include a near-field communication (NFC) receiver/transmitter to detect proximity to another device, such as a mobile phone. When the device is brought into close or detectable proximity to the second device, it may trigger the start of new functionality on the second device (e.g., the launching of an “app” on the mobile phone and radio syncing of physiological data from the device to the second device).

In another embodiment, the disclosed system includes a location sensor (for example, GPS circuitry) and heart rate sensor (for example, photoplethysmography circuitry) to generate GPS or location related data and heart rate related data, respectively. The disclosed system may then fuse, process and/or combine data from these two sensors/circuitry to, for example, determine, correlate and/or “map” geographical regions according to physiological data (for example, heart rate, stress, activity level, quantity of sleep and/or caloric intake). In this way, the disclosed system may identify geographical regions that increase or decrease a measurable user metric including but not limited to heart rate, stress, activity, level, quantity of sleep and/or caloric intake.

In addition thereto, or in lieu thereof, the disclosed system may employ the GPS related data and photoplethysmography related data (notably, each of which may be considered data streams), to determine or correlate the user's heart rate according to activity levels—for example, as determined by the user's acceleration, speed, location and/or distance traveled (as measured by the GPS and/or determined from GPS related data). Here, in one embodiment, heart rate as a function of speed may be “plotted” for the user, or the data could be broken down into different levels including but not limited to sleeping, resting, sedentary, moderately active, active, and highly active.

Indeed, the biometric monitoring device may also correlate GPS related data to a database of predetermined geographic locations that have activities associated with them for a set of predetermined conditions. For example, activity determination and corresponding physiological classification (for example, heart rate classification) may include correlating a user's GPS coordinates that correspond to location(s) of exercise equipment, health club and/or gym and physiological data. Under these circumstances, a user's heart rate during, for example a gym workout, may be automatically measured and displayed. Notably, many physiological classifications may be based on GPS related data including location, acceleration, altitude, distance and/or velocity. Such a database including geographic data and physiological data may be compiled, developed and/or stored on the biometric monitoring device and/or external computing device. Indeed, in one embodiment, the user may create their own location database or add to or modify the location database to better classify their activities.

In another embodiment, the user may simultaneously wear multiple devices. The devices may communicate with each other or a remote device using wired or wireless circuitry to calculate, for example, biometric or physiologic qualities or quantities that, for example, may be difficult or inaccurate to calculate otherwise such as pulse transit time. The use of multiple sensors may also improve the accuracy and/or precision of biometric measurements over the accuracy and/or precision of a single sensor. For example, having a device on the waist, wrist, and ankle could improve the detection of the user taking a step over that of a single device in only one of those locations. Signal processing could be performed on the devices in a distributed or centralized method to provide improved measurements over that of a single device. This signal processing could also be performed remotely and communicated back to the devices after processing.

### Processing Task Delegation

The disclosed system may include one or more processors. For example, an independent application processor may be used to store and execute applications that utilize sensor data acquired and processed by one or more sensor processors (processor(s) that process data from physiological, environmental and/or activity sensors). In the case where there are multiple sensors, there may also be multiple sensor processors. An application processor may have sensors directly connected to it as well. Sensor and application processors may exist as separate discrete chips or exist within the same packaged chip (multi-core). A device may have a single application processor, or an application processor and sensor processor, or a plurality of application processors and sensor processors.

In one embodiment, the sensor package could be placed on a daughterboard that consists of all of the analog components. This board may have some of the electronics typically found on the main PCB such as, but not limited to, transimpedance amplifiers, filtering circuits, level shifters, sample and hold circuits, and a microcontroller unit. Such a configuration may allow the daughterboard to be connected to the main PCB through the use of a digital connection rather than analog in addition to any necessary power or ground connections. A digital connection may have a variety of advantages over the analog daughter to main PCB connection including but not limited to a reduction in noise and a reduction in the number of necessary cables. The daughterboard may be connected to the main board through the use of a flex cable or set of wires.

Multiple applications can be stored on an application processor. An application can consist of executable code and data for the application, but not limited to these. Data can consist of graphics or other information required to execute the application or it can be information output generated by the application. The executable code and data for the application can both reside on the application processor or the data for the application can be stored and retrieved from an external memory. External memory may include but is not limited to NAND flash, NOR flash, flash on another processor, other solid-state storage, mechanical or optical disks, RAM.

The executable code for an application can also be stored on an external memory. When an application is requested to be executed, the application processor retrieves the executable code and/or data from the external storage and executes it. The executable code can be temporarily or permanently stored on the memory or storage of the application processor. This allows the application to be executed more quickly on the next execution request, since the step of retrieval is eliminated. When the application is requested to be executed, the application processor can retrieve all of the executable code of the application or portions of the executable code. In the latter case, only the portion of executable code required at that moment is retrieved. This allows applications that are larger than the application processor's memory or storage to be executed.

The application processor can also have memory protection features to prevent applications from overwriting, corrupting, interrupting, blocking, or otherwise interfering with other applications, the sensor system, the application processor, or other components of the system.

Applications can be loaded onto the application processor and any external storage via a variety of wired, wireless, optical, capacitive mechanisms including but not limited to USB, Wi-Fi, Bluetooth, Bluetooth Low Energy, NFC, RFID, Zigbee.

Applications can be cryptographically signed with an electronic signature. The application processor can restrict the execution of applications to those that have the correct signature.

### Methods of Wearing the Device

The disclosed system may include a housing having a size and shape that facilitates fixing the device to the user's body during normal operation wherein the device, when coupled to the user, does not measurably or appreciably impact the user's activity. The device may be worn in different ways depending on the specific sensor package integrated into the device and the data that the user would like to acquire.

A user may wear one or more of the disclosed system on their wrist or ankle (or arm or leg) with the use of a band that is flexible and thereby readily fitted to the user. The band may have an adjustable circumference, therefore allowing it to be fitted to the user. The band may be constructed from a material that shrinks when exposed to heat, therefore allowing the user to create a custom fit. The band may be detachable from the “electronics” portion of the biometric monitoring device and, if necessary, replaceable.

In an embodiment, the biometric monitoring device consists of two major components—a body (containing the “electronics”) and a band (that facilitates attaching the device to the user). The body may include a housing (made, for example, of a plastic or plastic-like material) and extension tabs projecting from the body (made, for example, from a metal or metal-like material). The band (made, for example, of a thermoplastic urethane) is attachable to the body mechanically or adhesively. The band may extend out a fraction of the circumference of the user's wrist. The distal ends of the urethane band may be connected with a Velcro, a hook and/or loop elastic fabric band that loops around a D-Ring on one side and then attaches back to itself. In this embodiment, the closure mechanism would allow the user infinite band length adjustment (unlike an indexed hole and mechanical clasp closure). The Velcro or fabric could be attached to the band in a manner that allows it to be replaced (for example, if it is worn or otherwise undesirable to wear before the useful or end of life of the device). In one embodiment, the Velcro or fabric would be attached with screws or rivets and/or glue, adhesive and/or clasp to the band.

The disclosed system may also be integrated and worn in a necklace, chest band, bra, patch, glasses, earring, or toe band. The device may be built in such a way that the sensor package/portion of the biometric monitoring device is removable and can be worn in any number of ways including, but not limited to, those listed above.

In another embodiment, the disclosed system may be worn clipped to an article of clothing or deposited in clothing (e.g., pocket) or an accessory (e.g., handbag, backpack, wallet). Because the biometric monitoring device may not be near the user's skin, in embodiments that include heart rate measurements, the measurements may be obtained in a discrete, “on demand” context by the user manually placing the device into a specific mode (e.g., depressing a button, covering a capacitive touch sensor, etc., possibly with the heart rate sensor embedded in the button/sensor) or automatically once the user places the device against the skin (e.g., applying the finger to an optical heart rate sensor).

User Interface with the Device

The disclosed system may include one or more methods of interacting with the device either locally or remotely.

In one embodiment, the disclosed system may convey data visually through a digital display. The physical embodiment of this display may use any one or a plurality of display technologies including, but not limited to one or more of LED, LCD, AMOLED, E-Ink, Sharp display technology, graphical display, and other display technologies such as TN, HTN, STN, FSTN, TFT, IPS, and OLET. This display could show data acquired or stored locally on the device or could display data acquired remotely from other devices or Internet services. The device may use a sensor (for example, an Ambient Light Sensor, “ALS”) to control or adjust screen backlighting. For example, in dark lighting situations, the display may be dimmed to conserve battery life, whereas in bright lighting situations, the display may increase its brightness so that it is more easily read by the user.

In another embodiment, the device may use single or multicolor LEDs to indicate a state of the device. States that the device indicate may include but are not limited to biometric states such as heart rate or application states such as an incoming message, a goal has been reached. These states may be indicated through the LED's color, being on, off, an intermediate intensity, pulsing (and/or rate thereof), and/or a pattern of light intensities from completely off to highest brightness. In one embodiment, an LED may modulate its intensity and/or color with the phase and frequency of the user's heart rate.

In an embodiment, the use of an E-Ink display would allow the display to remain on without the battery drain of a non-reflective display. This “always-on” functionality may provide a pleasant user experience in the case of, for example, a watch application where the user may simply glance at the device to see the time. The E-Ink display always displays content without comprising the battery life of the device, allowing the user to see the time as they would on a traditional watch.

The device may use a light such as an LED to display the heart rate of the user by modulating the amplitude of the light emitted at the frequency of the user's heart rate. The device may depict heart rate zones (e.g., aerobic, anaerobic) through the color of an LED (e.g., green, red) or a sequence of LEDs that light up in accordance with changes in heart rate (e.g., a progress bar). The device may be integrated or incorporated into another device or structure, for example, glasses or goggles, or communicate with glasses or goggles to display this information to the user. The disclosed system may also convey information to a user through the physical motion of the device. One such embodiment of a method to physically move the device is the use of a vibration inducing motor. The device may use this method alone, or in combination with a plurality of motion inducing technologies. The device may convey information to a user through audio. A speaker could convey information through the use of audio tones, voice, songs, or other sounds.

The disclosed system may be equipped with wireless and/or wired communication circuitry to display data on a secondary device in real time. For example, the disclosed system may be able to communicate with a mobile phone via Bluetooth Low Energy in order to give real-time feedback of heart rate, heart rate variability, and/or stress to the user. The disclosed system may coach or grant “points” for the user to breathe in specific ways that alleviate stress. Stress may be quantified or evaluated through heart rate, heart rate variability, skin temperature, changes in motion-activity data and/or galvanic skin response.

The disclosed system may receive input from the user through one or more local or remote input methods. One such embodiment of local user input could use a sensor or set of sensors to translate a user's movement into a command to the device. Such motions could include but may not be limited to tapping, rolling the wrist, flexing one or more muscles, and swinging. Another user input method may be through the use of a button of type, but not limited to the types, capacitive touch button, capacitive screen, and mechanical button. In one embodiment, the user interface buttons may be made of metal. In the case that the screen uses capacitive touch detection, it may always be sampling and ready to respond to any gesture or input without an intervening event such as pushing a physical button. The device may also take input through the use of audio commands. All of these input methods may be integrated into the device locally or integrated into a remote device that can communicate with the device either through a wired or wireless connection. In addition, the user may also be able to manipulate the device through a remote device. In one embodiment, this remote device could have Internet connectivity.

In one embodiment, the disclosed system may act as a wrist-mounted vibrating alarm to silently wake the user from sleep. The biometric monitoring device may track the user's sleep quality, waking periods, sleep latency, sleep efficiency, sleep stages (e.g., deep sleep vs REM), and/or other sleep-related metrics through one or a combination of heart rate, heart rate variability, galvanic skin response, motion sensing (e.g., accelerometer, gyroscope, magnetometer), and skin temperature. The user may specify a desired alarm time and the invention may use one or more of the sleep metrics to determine an optimal time to wake the user. In one embodiment, when the vibrating alarm is active, the user may cause it to hibernate or turn off by slapping or tapping the device (which is detected, for example, via motion sensor(s), a pressure/force sensor and/or capacitive touch sensor in the device). In one embodiment, the device may attempt to arouse the user at an optimum point in the sleep cycle by starting a small vibration at a specific user sleep stage or time prior to the alarm setting. It may progressively increase the intensity or noticeability of the vibration as the user progresses toward wakefulness or toward the alarm setting.

In another aspect, the disclosed system may be configured or communicated with using onboard optical sensors such as the components in an optical heart rate monitor.

### Wireless Connectivity and Data Transmission

The disclosed system may include a means of wireless communication to transmit and receive information from the Internet and/or other devices. The wireless communication may consist of one or more means such as Bluetooth, ANT, WLAN, power-line networking, and cell phone networks. These are provided as examples and do not exclude other wireless communication methods existent or that are yet to be invented.

The wireless connection is two ways. The device may transmit, communicate and/or push its data to other peripheral devices and/or the Internet. The device may also receive, request and/or pull data from other peripheral devices and/or the Internet.

The disclosed system may act as a relay to provide communication for other devices to each other or to the Internet. For example, the device may connect to the Internet via WLAN but also be equipped with an ANT radio. An ANT device may communicate with the device to transmit its data to the Internet through the device's WLAN (and vice versa). As another example, the device may be equipped with Bluetooth. If a Bluetooth-enabled smart phone comes within reach of the device, the device may transmit data to or receive data from the Internet through the smart phone's cell phone network. Data from another device may also be transmitted to the device and stored (and vice versa) or transmitted at a later time.

The disclosed system may also include streaming or transmitting web content for displaying on the biometric monitoring device.

Content may be delivered to the disclosed system according to different contexts. For instance, in the morning, news and weather reports may be displayed along with the user's sleep data from the previous night. In the evening, a daily summary of the day's activities may be displayed.

The disclosed system may also include NFC, RFID, or other short-range wireless communication circuitry that may be used to initiate functionality in other devices. For instance, the disclosed system may be equipped with an NFC antenna so that when a user puts it into close proximity with a mobile phone, an app is launched automatically on the mobile phone.

### Charging and Data Transmission

The disclosed system may use a wired connection to charge an internal rechargeable battery and/or transfer data to a host device such as a laptop or mobile phone. In one embodiment, the device may use magnets to help the user align the device to the dock or cable. The magnetic field of magnets in the dock or cable and the magnets in the device itself could be strategically oriented to as to force the device to self-align and provide a force that holds the device to the dock or cable. The magnets may also be used as conductive contacts for charging or data transmission. In another embodiment, a permanent magnet is only used in the dock or cable side, not in the device itself. This may improve the performance of the biometric monitoring device where the device employs a magnetometer. With a magnet in the device, the strong field of a nearby permanent magnet may increase the difficulty for the magnetometer to accurately measure the earth's magnetic field.

In another embodiment, the device could contain one or more electromagnets in the device body. The charger or dock for charging and data transmission would also contain an electromagnet and/or a permanent magnet. The device could only turn on its electromagnet when it is close to the charger or dock. It could detect proximity to the dock by looking for the magnetic field signature of a permanent magnet in the charger or dock using a magnetometer. Alternatively it could detect proximity to the charger by measuring the Received Signal Strength Indication or RSSI of a wireless signal from the charger or dock. The electromagnet could be reversed, creating a force that repels the device from the charging cable or dock either when the device doesn't need to be charged, synced, or when it has completed syncing or charging.

### Configurable App Functionality

In some embodiments, the disclosed system may include a watch-like form factor and/or bracelet, armlet, or anklet form factor and may be programmed with “apps” that launch specific functionality and/or display specific information. Apps may be launched or closed by a variety of means including but not limited to pressing a button, using a capacitive touch sensor, performing a gesture that is detected by an accelerometer, moving to a location detected by a GPS or motion sensor, compressing the device body, thereby creating a pressure signal inside the device that is detected by an altimeter, or placing the device close to an NFC tag which is associated with an app or set of apps. Apps may also be automatically triggered to launch or close by certain environmental or physiological conditions including but not limited to a high heart rate, the detection of water using a wet sensor (to launch a swimming application for example), a certain time of day (to launch a sleep tracking application at night for example), a change in pressure and motion characteristic of a plane taking off or landing to launch and close an “airplane” mode app. Apps may also be launched or closed by meeting multiple conditions simultaneously. For example, if an accelerometer detects that a user is running and the user presses a button it may launch a pedometer application, an altimeter data collection application and/or display. In another case where the accelerometer detects swimming and the user presses the same button, it may launch a lap counting application.

In one embodiment, the device could have a swim-tracking mode that may be launched by starting a swimming app. In this mode, the device's motion sensors and/or magnetometer may be used to detect swim strokes, classify swim stroke types, detect swimming laps, and other related metrics such as stroke efficiency, lap time, speed, distance, and calorie burn. Directional changes indicated by the magnetometer may be used to detect a diversity of lap turn methods. In a preferred embodiment, data from a motion sensor and/or pressure sensor may be used to detect strokes.

In another embodiment, a bicycling app may be launched by moving the device within proximity of an NFC or RFID tag that is located on the bicycle, on a mount on the bicycle or in a location associated with a bicycle including but not limited to a bike rack or bike storage facility. The app launched may use a different algorithm than is normally used to determine metrics including but not limited to calories burned, distance traveled, and elevation gained. The app may also be launched when a wireless bike sensor is detected including but not limited to a wheel sensor, GPS, cadence sensor, or power meter. The device may then display and/or record data from the wireless bike sensor or bike sensors.

Additional apps include but are not limited to a programmable or customizable watch face, stop watch, music player controller (e.g., mp3 player remote control), text message and/or email display or notifier, navigational compass, bicycle computer display (when communicating with a separate or integrated GPS device, wheel sensor, or power meter), weight lifting tracker, sit-up reps tracker, pull up reps tracker, resistance training form/workout tracker, golf swing analyzer, tennis (or other racquet sport) swing/serve analyzer, tennis game swing detector, baseball swing analyzer, ball throw analyzer (e.g., football, baseball), organized sports activity intensity tracker (e.g., football, baseball, basketball, volleyball, soccer), disk throw analyzer, food bite detector, typing analyzer, tilt sensor, sleep quality tracker, alarm clock, stress meter, stress/relaxation biofeedback game (e.g., potentially in combination with a mobile phone that provides auditory and/or visual cues to train user breathing in relaxation exercises), teeth brushing tracker, eating rate tracker (e.g., to count or track the rate and duration by which a utensil is brought to the mouth for food intake), intoxication or suitability to drive a motor vehicle indicator (e.g., through heart rate, heart rate variability, galvanic skin response, gait analysis, puzzle solving, and the like), allergy tracker (e.g., using galvanic skin response, heart rate, skin temperature, pollen sensing and the like, possibly in combination with external seasonal allergen tracking from, for instance, the internet; possibly determining the user's response to particular forms of allergen (e.g., tree pollen) and alerting the user to the presence of such allergens (e.g., from seasonal information, pollen tracking databases, or local environmental sensors in the device or employed by the user)), fever tracker (e.g., measuring the risk, onset, or progress of a fever, cold, or other illness, possibly in combination with seasonal data, disease databases, user location, and/or user provided feedback to assess the spread of a particular disease (e.g., flu) in relation to a user, and possibly prescribing or suggesting the abstinence of work or activity in response), electronic games, caffeine affect tracker (e.g., monitoring the physiologic response such as heart rate, heart rate variability, galvanic skin response, skin temperature, blood pressure, stress, sleep, and/or activity in either short term or long term response to the intake or abstinence of coffee, tea, energy drinks and/or other caffeinated beverages), drug affect tracker (e.g., similar to the previously mentioned caffeine tracker but in relation to other interventions, whether they be medical or lifestyle drugs such as alcohol, tobacco, etc.), endurance sport coach (e.g., recommending or prescribing the intensity, duration, or profile of a running/bicycling/swimming workout, or suggesting the abstinence or delay of a workout, in accordance with a user specified goal such as a marathon, triathlon, or custom goal utilizing data from, for instance, historical exercise activity (e.g., distance run, pace), heart rate, heart rate variability, health/sickness/stress/fever state), weight and/or body composition, blood pressure, blood glucose, food intake or caloric balance tracker (e.g., notifying the user how many calories he may consume to maintain or achieve a weight), pedometer, and nail biting detector. In some cases, the apps may rely solely on the processing power and sensors of the invention. In other cases, the apps may fuse or merely display information from an external device or set of external devices including but not limited to a heart rate strap, GPS distance tracker, body composition scale, blood pressure monitor, blood glucose monitor, watch, smart watch, mobile communication device such as a smart phone or tablet, or server.

In one embodiment, the device may control a music player on a secondary device. Aspects of the music player that may be controlled include but are not limited to the volume, selection of tracks and/or playlists, skipping forward or backward, fast forwarding or rewinding of tracks, the tempo of the track, and the music player equalizer. Control of the music player may be via user input or automatic based on physiological, environmental, or contextual data. For example, a user may be able to select and play a track on their smart phone by selecting the track through a user interface on the device. In another example, the device may automatically choose an appropriate track based on the activity level of the user (the activity level being calculated from device sensor data). This may be used to help motivate a user to maintain a certain activity level. For example, if a user goes on a run and wants to keep their heart rate in a certain range, the device may play an upbeat or higher tempo track if their heart rate is below the range which they are aiming for.

### Location/Context Sensing and Applications

The disclosed system may have sensors that can determine or estimate the location and or context (e.g. in a bus, at home, in a car) of the user. Purpose-built location sensors such as GPS, GLONASS, or other GNSS (Global Navigation Satellite System) sensors may be used. Alternatively, location may be inferred, estimated or guessed using less precise sensors. In some embodiments in which it is difficult to know the user's location, user input may aid in the determination of their location and or context. For example, if sensor data makes it difficult to determine if a user was in a car or a bus, the biometric monitoring device or a portable communication device in communication with the biometric monitoring device or a cloud server which is in communication with the biometric monitoring device may present a query to the user asking them if they took the bus today or took a car. Similar queries may occur for locations other than vehicular contexts. For example, if sensor data indicate that the user completed a vigorous workout, but there is no location data that indicates that the user went to a gym, the user may be asked if they went to the gym today.

### EXAMPLES

Certain materials or components of the devices, systems, and methods described herein may be made by known materials or methods or may be commercially available. It is also possible to make use of variants which are themselves known to those of ordinary skill in this art but are not mentioned in greater detail. The skilled artisan given the literature and this disclosure is well equipped to prepare the formulations of the instant application using either the hardware, software, learning or combinations thereof.

### Organization of Examples

Examples involve all aspects of biology and synthetic biology. The four exemplary cases/examples relate to human, non-human, synthetic and models of the aforementioned. The disclosed technology provides new ways to measure health capacity, new ways to use these ways to learn about health, and such learning permits new modes of improving health, including:
1. Case 1. Humans: In humans, the disclosed technology measures/quantifies to diagnose, and used the results of diagnose is to treat. The disclosed technology diagnoses disease and detects the absence of health. “Treat,” as used herein, is defined broadly to include prophylaxis through recovery, and includes the optimization (maximization) of health and/or the interception of disease. Human use will include all tissues, cells and organs etc. that are derived from humans to “treat.”
2. Case 2, Non-Humans: In the case of non-human application, this includes the entirety of biology that is non-human. Other animals, plants, single cell organisms. For non-humans, the observed systems are not merely diagnosed and/or treated, but rather can be “measured” and “controlled.” In this context, measure can be quantifying any representation of the energy budget.
3. Case 3, Industrial/Synthetic Biology: In the case of genetic engineering for industrial biology or synthetic biology the observed systems are designed and engineered. Knowledge of the energy budget or any representation of that budget, permits “designing” of the observed system, Such designing brings about a biochemical transformation (work) that was not present in the native (wild type) species. One benefit derived from such designing, is that the disclosed technology permits “engineering” of the observed system. Engineering refers to any method of bringing about the design intention.
4. Case 4, Models of Humans, Non-Humans, Synthetic/Industrial: In the case of models, knowledge of the energy budget derived from Human, Non-Human or Synthetic/Industrial systems is used to design and build, in a laboratory or other contrived setting or in silico, an instantiation of the system or some part, component, module thereof, capable of testing sequences of intervention for the purpose of learning and validating methods of treatment (interception), optimization, or engineering of the real world counterpart.
5. Case 5, Other: Knowledge of the energy budget of any system may be used to improve encryption, by using energy signature information or any energy signature-based representation. It may be used to improve ecology, especially in the context of carbon credit trading. It may be used in the context of the insurance industry especially with respect to implementing real time risks allocation and pricing. It may be used in the field of cybernetic, especially in the context if human/machine interfaces. It may be used in the field of art, especially with respect to expressions of energy signatures. It may be used in the gaming industry, especially as biomimetics may be used that are based on energy budget rules.

### Example 1

### Quantification and Optimization of Human Health and Early Diagnosis, Detection, and Treatment and Interception of Disease

### Characteristics of Device

The disclosed technology provides, in one embodiment, a low latency, automated wearable measurement device and platform that is configured to quantify indicators of health and to detect patterns of change of health before symptoms that enable the pre-symptomatic detection and interception of disease. This embodiment is, preferably, simple to use, automated, safe, precise, and accurate. A long battery life will enable continuous and uninterrupted measurement. The battery is configured to provide more than 100 day, more than 200 day, more than 300 day, and preferably approximately 360 day always-on, non-rechargeable requirement to provide real-time individual and population-based analytics at a global scale. To achieve this, parameters are selected that can be detected by sensors with low power requirements controlled by firmware algorithms that automatically adjust sampling rate and frequency based on pre-set thresholds or changes in baseline.

In a preferred embodiment, a device will measure heat flux in 20 second intervals, and in subsequent devices additional sensors will be onboarded to improve the precision and accuracy of heat flux determination, and in subsequent versions the addition of sensors to quantify work.

In a preferred embodiment Device #1 will sense changes in cellular heat and work and accompanying software platform will analyze this data for individual and at scale. Despite the near infinite (millions) of ways a cell uses energy, there are only a finite number of ways a cell expends energy. Specifically, all changes in cellular energy usage are captured in/by two parameters: changes in heat (ΔQ) and work (ΔW).

In some embodiments, the platform contains hardware that senses changes in cellular heat and work and streams this data to our software which stores, analyzes and displays in real-time how energy is allocated among heat and various types of work—the “energy budget.” Changes in allocations, frequency, amplitude or rate of change of the energy signature precede standard vital signs and are information rich-increasing their value for learning.

The approach used in the disclosed technology takes into account the gold-standard measurements made in the clinic and laboratory of human heat and work. Clinically, such measurements are made using direct or indirect calorimetry. Both of these methods are highly accurate and precise but are limited by portability. Similarly, in the laboratory, analogous measurements are made, but they too require sophisticated centralized equipment. To overcome these measurement limitations, we reframed the problem: what measurements of heat and work could be made by a suite of miniaturized sensors that parameterize cell energy, and readily and continuously streamed for long periods of time (>180 days) to a learning engine enabling the pre-symptomatic detection and interception of disease and learning at scale?

Sensor selection takes into account known values of human energy expenditures to their allocations on a cellular level. As the majority of cellular energy is expended producing heat and in active transport of ions and water, some embodiments focus on sensing and parametrizing these properties as the basis of Device #1. In some embodiments, the sensors of Device #1 also contain relative humidity, barometric pressure and light sensors.

In some embodiments, the sensors are selected based on the following estimations of energy budget: (1) At a physiologic scale, the total energy expenditure is the sum of resting energy, diet energy and physical energy: Total Energy Expenditure (EE)=Resting EE+Physical Activity EE+Diet EE. This is a physiologic energy budget; (2) The resting energy expenditure is roughly 80% of total energy expenditure: REE˜80% EE; (3) Humans are inefficient machines in that ˜60% of energy is lost as heat and only 40% captured as work: EE ˜60% ΔQ+40% ΔW; (4) The work done within cells is largely that of moving water and ions, synthesizing proteins and biochemical (intermediary metabolism). Therefore ΔW is estimated at 25% ion movement; 25% structure; 50% biochemical; (5) The heat produced within cells is the same heat produced by organs and the same heat detected leaving the body through the skin. Therefore ΔQ is the same; (6) Ideally a device would quantify all work done, however the V1.0 device focuses on sensors that were largely commercially available; and (7) As there are sensors commercially available that allow quantifying heat flux and work associated with water, the accuracy of the Device #1 is estimated to be at ˜85% of the total ΔE or Energy Expenditure. This accuracy is sufficient to quantify an energy budget and capture work done involving water* to quantify and optimize health and pre-symptomatically detect and intercept disease.

Attributes of this example of a device include:
1. Battery Life: The battery life of the device is at least 180 days, batteries are disposable, and internally powered by a standard 3-Volt (peak recharge) lithium coin cell battery of 50 mAh total capacity. This allows for continuous measurement over long period of time;
2. Usability: The device is rugged, inexpensive, and simple to use which allows for widespread deployment in hospital, assisted care, and ambulatory medical settings;
3. Software: The device readily interfaces to the IoT (cloud/machine learning) which allows for the automation of data recording and analysis;
4. Simple: The device requires no specialized skills to operate. This allows for widespread deployment of a decentralized health measurement system;
5. Inexpensive: A commercial cost of <$100.00 is estimated. This lowers financial hurdles to broad deployment;
6. Automated: The device measures your health continuously which allows for early detection of change before disease symptoms and allows for the generation of big data sets to detect and learn during population-based health threats;
7. Rugged Design Standards: The device is designed to operate reliably in a harsh environment and/or harsh conditions. This allows for deployment in diverse civilian, first-responder, and warfighter settings;
8. Secure: All communication between the device and smartphone are done over an encrypted Bluetooth Low Energy link, specifically “LESC”. No identity information about the user is stored on the wrist sensor or transmitted unencrypted by Bluetooth. The connection between the app and cloud storage servers is secure. At each stage the data is encrypted at rest (on device or cloud) and during transmission. Protected Health Information will be gathered and moved to secure cloud only with consent of the device wearer. Access to data in cloud for purposes of analytics development will reveal only formally de-identified data;
9. Safe: The sensor module is an external skin surface applied battery powered sensor suite contained within an isolated polymer (Delrin) encasement with only non-electrically conductive surface materials of exposure. There are no electrical contacts between the internal electrical circuitry and the skin.

### Device Hardware

The device is, in a preferred embodiment, an integrated miniaturized wireless sensor suite based on an FCC-approved BMD-350 SoC (System on a Chip) using a Nordic NRF52 microcontroller, a chip antenna, with bidirectional communication enabling alerts to be provided to an individuals or healthcare provider with a smartphone (app) via Bluetooth Low Energy (BLE). The BLE connection is encrypted using LESC (“low energy secure communication”), as defined in the Bluetooth Core Specification v4.2 (and later). Communication back to the device is used to change sampling rates and service the device, such as updating firmware. The Surface Mount (SMT) circuit is comprised of the SoC, two digital temperature sensors, and a digital accelerometer reporting to the microcontroller, and an EPPROM with standard support circuitry SMT components such as resistors and filtering capacitors, and two chip LEDs. Power is provided by a non-rechargeable Renata CR1616 coin cell lithium battery with total 50 mAh capacity. Internal materials of the PCB are standard FR4 rigid and polyimide flex circuitry, solder is lead-free, and two tiny stainless steel 000 size (like eyeglass size) screws that hold the PCB into place within the encasement. The encasement and materials of exposure are machined Delrin (encasement, 0.53 square inches), black anodized aluminum (thermal disc, 0.06 square inches), gold-plated aluminum (heat exchange ring) and a small Corning Gorilla glass sight window (same as used in iPhone screens). Data is pre-processed within the firmware on board the sensor before transmitting to the phone. The phone app displays, and stores data transmitted from the sensor. The wireless output from the device is timestamp, air (ambient) temp, skin temp, acceleration data, error state (if any), and the device battery voltage all transmitted to the phone via BLE. No data will be provided to the patient or individual with the device. As currently designed, all data will only be available to medical personnel through access to the smartphone app.

Sensor System #1 is a wearable sensor-containing system that continuously and accurately measures and obtains data relating to thermal flux. It offers significant advantages over existing alternatives, including the potential, compared to existing approved alternatives, to reduce or eliminate the need for hospitalization, improve patient quality of life, facilitate patients' ability to manage their own care (such as through self-directed personal assistance), or establish long.

Sensor System #1, in certain embodiments, a miniaturized indirect calorimeter that continuous measures metabolic rate over 300 days at 1 second intervals to enable the pre-symptomatic detection of infection.

Pathogens cause a host hypermetabolic response resulting in fever. [For every 1 degree C. change in core body temperature, energy expenditure changes by ˜13%.] As changes in metabolic rate that result in heat production precede changes in core temperature (“fever”) Sensor Alpha works by detecting infection before conventional thermometry.

Detection of infection enabling interception by real-time indirect calorimetric measurement of metabolic rate.

Sensor System #1 is differentiated from known sensor-based systems by at least the following criteria:

Hardware Design: Sensor System #1, in certain embodiments, is configured to simultaneously measures both ambient and skin temperatures which is essential to accurate real time quantification of heat flow and metabolic rate.

Skin temperature is determined by the combination of two thermal contributions, namely the external (ambient) and internal (metabolic). This can be expressed by the equation T_skin=T_ambient+T_metabolic, where T_metabolic is a contribution to skin temperature supported by the metabolic heat of the body. By Newton's law of cooling the metabolic rate of a body, MR, will follow the proportionality, MR=k (T_skin-T_ambient)=k T_metabolic. So an approximate, direct calorimetry can be achieved by measuring the difference between skin temperature and adjacent ambient temperature. An approximation to this relationship which treats ambient temperature as a constant or some other simple model, might be attempted. However, skin and air temperature are highly correlated on short time scales due to counter current exchange processes. Any model which attempts to estimate heat flux without a local measure of ambient temperature will be highly inaccurate. Said in other terms, the variance of ambient temperature is typically much greater than the variance of the skin-to-air temperature difference. Empirically, we find that the fluctuation scale of ambient temperature is roughly 3 times greater than the scale of T_metabolic even in moderate climates. If variance due to ambient temperature is not carefully removed it will entirely obscure the variance due to metabolic processes. Therefore, simultaneous measurement of skin and ambient temp is critical to the performance this sort of calorimetry.

TABLE 5
Comparison of Sensor Alpha with other methods
of heat flux and temperature determination.

| | | Metabolic | | |
|---|---|---|---|---|
| Method | Quantifies | Rate | Continuous | Scalable |
| Direct calorimetry | Heat flux | Yes | No | No |
| Indirect calorimetry | O₂/CO₂ | Yes | No | No |
| Thermometry | Temperature | No | Yes | Yes |
| Thermography | Temperature | No | No | Yes |
| Sensor System #1 | Heat flux | Yes | Yes | Yes |
| (Direct | | | | |
| Approximated | | | | |
| Calorimetry) | | | | |

### Platform Software: Learning Engine

### Learning the Rules of Health Capacity

The rules governing the emergence of health capacity in biological systems can, in a preferred embodiment, be obtained based upon a training set and implemented by machine readable instructions. For example, the training sets is broken down into correlations between specific metrics, evaluated over time, and compared to independent health assessments of the individuals evaluated. High correlations between specific metrics and health assessments are reinforced, while poor correlations between specific metric and health assessments are given less weight. Interavtive reinforcement and re-weighing of the metrics is used to generate correlations, and those correlations are reduced to machine readable instructions for assessing future metrics. Health capacity may not be directly measurable, nor may be the rules around it. However, based on continuous measurement of biological energy expenditures, an understanding of health capacity and its quantification can, in a preferred embodiment, be obtained. In particular, the rules which govern the emergence of health capacity in biological systems can be obtained by quantifying various representations of health capacity or derivatives thereof which are intimately related to the function of biological systems. Such representations include energy budgets and signatures.

### Energy Signature/Budget as Representations of Health Capacity

The biological processes which generate heat do so according to the logic of an energy budget. That energy budget affords some health capacity. The energy signature represents the effect of the health capacity in response to ongoing stresses and circumstances. By predicting the energy signature, ahead of time, the systems and methods disclosed herein offer a model of internal processes which is accurate and correlated with health capacity. These systems and methods are capable of predicting health and disease outcomes and persistence. Energy signature is predictable from past energy signature or from an energy budget, therefore each are useful as representations of health capacity.

### Annotations, Metabolic Tasks and the Key Determinants of Health

Sleep, diet, exercise, and lifestyle are observed to be the key determinants of health (KDOH). Each of these determinants may involve several overlapping metabolic tasks. Positive engagement with KDOH must involve a mixture of metabolic tasks which are commensurate with human energy budgets. By contrast, poor engagement with kDoH results in a set of metabolic tasks which are incommensurate with human energy budget. We therefore center our annotation strategy on the resolution of the KDOH where human control resides. Annotation will occur primarily at the level of KDOH. Higher resolution annotations at the level of individual metabolic tasks and underlying mechanisms may be used to enhance learning.

### Platform Software: Learning Engine

The technology platform is equipped with a learning engine, mobile application software, a cloud infrastructure capable of continuously ingesting data from our proprietary sensor hardware for the purpose of description, prediction, and inference regarding biological systems. The ingested data will include energy measurements and auxiliary sensor data (biological or environmental) which are intended to characterize metabolic tasks and other biological processes to aid in the quantification of energy expenditures and signatures. The learning engine will execute the following functions:
Quantify energy signatures;
Correlate energy signatures with function, disease, outcome;
Infer the rules of an energy budget through relationship of annotation and energy signature;
Correlate energy budget with function, disease, outcome;
Refine and test energy budget quantification via app-based recommendation and feedback;
Infer the health capacity associated with an energy budget under various stresses as indicated by the probability of persistence;
Identify the critical regularities within energy budgets which are necessary and sufficient conditions for Health Capacity: the rules of Health Capacity;
Identification of ‘energy gaps’ as evidence of the need for an additional annotation or sensor.

### Software Learning Strategies

Our technical challenge is to extract simple energy budget rules from the complexity of continuous energy signatures. To do so we have developed a learning strategy in two parts. First, we associate time stamped annotation with KDOH to relate energy expenditure with critical metabolic tasks. By learning patterns in these temporal relationships, we can infer an energy budget which describes the predominant components of the energy signature. Second, with these principal components annotated and quantified, we can detect, using sustain, continuous monitoring, previously unseen anomalies in energy signature which have not previously been detected and explained. Through a process of successive approximation involving new annotations and sensors, the energy signature will be described and predicted with increasing completeness and accuracy. As our energy budget models become increasingly accurate and our dataset grows, unstable regions of the energy budget space or areas of low or zero health capacity will be able to be identified. The boundaries of the region of finite Health Capacity will be determined by the rules of Health Capacity. The rules of Health Capacity may be inferred through examination/analysis of species, individuals, disease states which exist along the boundary, to determine their primary weakness.

### Energy Budget Conceptualization and Visualization

Many biological processes perform work which is not directly measurable, but that work may also show up later as waste heat. Take as an example the case of the circulatory system which involves multiple stages of energy transformation. A careful energy audit would include the following Steps: (Step 1) breaking chemical bonds to make ATP in cardiac tissue (work and heat); (Step 2) use ATP to contract cardiac muscle (work); (Step 3) blood pushed through vasculature against viscosity/friction (heat); and (Step 4) oxygen reaches periphery allowing mitochondria to run oxidative phosphorylation (heat and work). Steps 2, and 3 involve energy which has already been counted in Step 1. The energy has an origin (glucose metabolism), an intermediate form (kinetic energy of blood) and a final form (exhaust heat). Steps 2 and 3 will be somewhat decreased relative to Step 1 because there is always an energy conversion efficiency less than 100%. Step 4 comes as a consequence of Steps 1, 2, 3 but it is a distinct expenditure in the energy budget which involves distinct fuel sources. Nonetheless, the energy generated in Step 4 will be proportional to the oxygen delivered so there may be useful correlations to exploit.

To appropriately segment the energy budget without double counting energy is a primary technical challenge of this effort. The most general statement of the double counting problem is that energy can be measured at various points in a flow and that misalignments of relationships can result in double counting. Triangulating on different components of energy with semi-redundant measurement will allow constructing a Sankey-type map of energy flow.

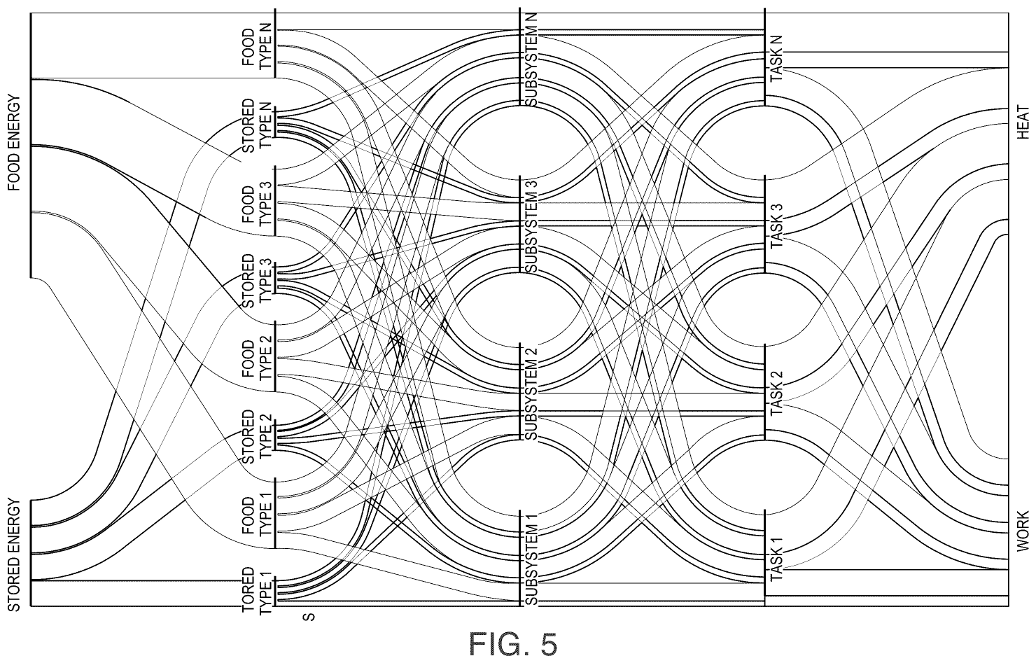

In particular, FIG. 5 (and also FIGS. 11 and 12) illustrate that, by charting out the flow of energy functionally, the disclosed technology can simultaneously avoid double-counting and learn the structure of energy relationships. Therefore, sensors and metadata collection can be chosen which transect the Sankey diagram in diverse ways. Each additional measurement adds resolution to the flow map.

### Generalized Health Learning Loop

In some embodiments, the disclosed technology uses a low latency, automated wearable measurement device for generalized health learning. The device may measure the baseline energy signature and continuously quantifies baseline variance associated with annotations. In some embodiments, the disclosed technology further uses a generalized health learning platform to quantify indicators of health and to construct a personal knowledge base of quantified health interventions (e.g., effectiveness of various foods to shift a subject's energy expenditure). In some embodiments, the platform further make real time recommendations based on current energy signature, personal energy budget, and catalogued intervention knowledge base.

The device and the platform for generalized health learning are, preferably, simple to use, automated, safe, precise, and accurate. A long battery life associated with the device will enable continuous and uninterrupted measurement. The battery is configured to provide more than 100 day, more than 200 day, more than 300 day, and preferably approximately 360 day always-on, non-rechargeable requirement to provide real-time individual and population-based analytics at a global scale. To achieve this, parameters are selected that can be detected by sensors with low power requirements controlled by firmware algorithms that automatically adjust sampling rate and frequency based on pre-set thresholds or changes in baseline. In a preferred embodiment, the device will measure heat flux in 20 second intervals, and in subsequent devices additional sensors may be added to improve the precision and accuracy of heat flux determination, and in subsequent versions the addition of sensors to quantify work. In a preferred embodiment the device will sense changes in cellular heat and work and accompanying software platform will analyze this data for individual and at scale. The changes in cellular energy usage are captured in/by two parameters: changes in heat (ΔQ) and work (ΔW).

In some embodiments, the device for generalized health learning contains hardware that senses changes in cellular heat and work and streams this data to software which stores, analyzes and displays in real-time how energy is allocated among heat and various types of work—the “energy budget.” Changes in allocations, frequency, amplitude or rate of change of the energy signature precede standard vital signs and are information rich-increasing their value for learning.

In some embodiments, the device for generalized health learning comprises a suite of miniaturized sensors that parameterize cell energy, and readily and continuously streamed for long periods of time (>180 days) to a learning engine enabling construction of a personal knowledge base of quantified health interventions and real time recommendations based on current energy signature, personal energy budget, and catalogued intervention knowledge base. In some embodiments, the sensors may contain relative humidity, barometric pressure and light sensors. In some embodiments, the sensors may take into account known values of human energy expenditures to their allocations on a cellular level, or how cellular energy is expended producing heat and in active transport of ions and water.

In some embodiments, the sensors of the generalized health learning device are selected based on the following estimations of energy budget: (1) At a physiologic scale, the total energy expenditure is the sum of resting energy, diet energy and physical energy: Total Energy Expenditure (EE)=Resting EE+Physical Activity EE+Diet EE. This is a physiologic energy budget; (2) The resting energy expenditure is roughly 80% of total energy expenditure: REE˜80% EE; (3) Humans are inefficient machines in that ˜60% of energy is lost as heat and only 40% captured as work: EE˜60% ΔQ+40% ΔW; (4) The work done within cells is largely that of moving water and ions, synthesizing proteins and biochemical (intermediary metabolism). Therefore ΔW is estimated at 25% ion movement; 25% structure; 50% biochemical; (5) The heat produced within cells is the same heat produced by organs and the same heat detected leaving the body through the skin. Therefore ΔQ is the same; (6) Ideally a device would quantify all work done, however the V1.0 device focuses on sensors that were largely commercially available; and (7) As there are sensors commercially available that allow quantifying heat flux and work associated with water, the accuracy of the Device #1 is estimated to be at ˜85% of the total ΔE or Energy Expenditure. This accuracy is sufficient to quantify an energy budget and capture work done involving water* to quantify and optimize health and pre-symptomatically detect and intercept disease.

In some embodiments, attributes of the device for generalized health learning may include:
1. Battery Life: The battery life of the device is at least 180 days, batteries are disposable, and internally powered by a standard 3-Volt (peak recharge) lithium coin cell battery of 50 mAh total capacity. This allows for continuous measurement over long period of time;
2. Usability: The device is rugged, inexpensive, and simple to use which allows for widespread deployment in hospital, assisted care, and ambulatory medical settings;
3 Software: The device readily interfaces to the IoT (cloud/machine learning) which allows for the automation of data recording and analysis;
4. Simple: The device requires no specialized skills to operate. This allows for widespread deployment of a decentralized health measurement system;
5. Inexpensive: A commercial cost of <$100.00 is estimated. This lowers financial hurdles to broad deployment;
6. Automated: The device measures your health continuously which allows for early detection of change before disease symptoms and allows for the generation of big data sets to detect and learn during population-based health threats;
7. Rugged Design Standards: The device is designed to operate reliably in a harsh environment and/or harsh conditions. This allows for deployment in diverse civilian, first-responder, and warfighter settings;
8. Secure: All communication between the device and smartphone are done over an encrypted Bluetooth Low Energy link, specifically “LESC”. No identity information about the user is stored on the wrist sensor or transmitted unencrypted by Bluetooth. The connection between the app and cloud storage servers is secure. At each stage the data is encrypted at rest (on device or cloud) and during transmission. Protected Health Information will be gathered and moved to secure cloud only with consent of the device wearer. Access to data in cloud for purposes of analytics development will reveal only formally de-identified data;
9. Safe: The sensor module is an external skin surface applied battery powered sensor suite contained within an isolated polymer (Delrin) encasement with only non-electrically conductive surface materials of exposure. There are no electrical contacts between the internal electrical circuitry and the skin.

In some embodiments, the platform for generalized health learning is equipped with a learning engine, mobile application software, a cloud infrastructure capable of continuously ingesting data from the proprietary sensor hardware for the purpose of description, prediction, and inference regarding health. The ingested data will include energy measurements and auxiliary sensor data (biological or environmental) which are intended to characterize metabolic tasks and other biological processes to aid in the quantification of energy expenditures and signatures. The learning engine will execute the following functions:
Quantify energy signatures;
Correlate energy signatures with function, health, outcome;
Infer the rules of an energy budget through relationship of annotation and energy signature;
Correlate energy budget with function, health, outcome;
Refine and test energy budget quantification via app-based recommendation and feedback;
Infer the health capacity associated with an energy budget under various stresses as indicated by the probability of persistence;
Identify the critical regularities within energy budgets which are necessary and sufficient conditions for Health Capacity: the rules of Health Capacity;
Identification of ‘energy gaps’ as evidence of the need for an additional annotation or sensor.

### Generalized Disease Learning Loop

In some embodiments, the disclosed technology uses a low latency, automated wearable measurement device for generalized disease learning. In some embodiment, the device may perform measurement of the baseline energy signature. In some embodiments, the disclosed technology further comprises a generalized disease learning platform that detect patterns of change of health before symptoms that enable the pre-symptomatic detection and interception of disease. The generalized disease learning platform may perform continuous analysis of health signature to detect anomalies in real time, construction of disease anomaly knowledge base (e.g., pathogen or disease with signature anomaly and associated contextual risk factors), real time cross reference of personal anomalous signature with anomaly knowledge base, and/or near real time alerts to follow disease intervention guidance (e.g., precision testing).

The device and platform for generalized disease learning are, preferably, simple to use, automated, safe, precise, and accurate. A long battery life associated with the device will enable continuous and uninterrupted measurement. The battery is configured to provide more than 100 day, more than 200 day, more than 300 day, and preferably approximately 360 day always-on, non-rechargeable requirement to provide real-time individual and population-based analytics at a global scale. To achieve this, parameters are selected that can be detected by sensors with low power requirements controlled by firmware algorithms that automatically adjust sampling rate and frequency based on pre-set thresholds or changes in baseline.

In a preferred embodiment, the device for generalized disease learning will measure heat flux in 20 second intervals, and in subsequent devices additional sensors will be added to improve the precision and accuracy of heat flux determination, and in subsequent versions the addition of sensors to quantify work. In a preferred embodiment, the device for generalized disease learning will sense changes in cellular heat and work and accompanying software platform will analyze this data for individual and at scale. In some embodiment, changes in cellular energy usage are captured in/by two parameters: changes in heat (ΔQ) and work (ΔW).

In some embodiments, the device for generalized disease learning contains hardware that senses changes in cellular heat and work and streams this data to the software which stores, analyzes and displays in real-time how energy is allocated among heat and various types of work—the “energy budget.” Changes in allocations, frequency, amplitude or rate of change of the energy signature precede standard vital signs and are information rich-increasing their value for learning.

In a preferred embodiment, the device for generalized disease learning may perform measurements of heat and work made by a suite of miniaturized sensors that parameterize cell energy, and readily and continuously streamed for long periods of time (>180 days) to a learning engine enabling the pre-symptomatic detection and interception of disease and learning at scale.

In some embodiments, the sensors of the device for generalized disease learning may contain relative humidity, barometric pressure and light sensors. In some embodiments, the sensors may take into account known values of human energy expenditures to their allocations on a cellular level, or how cellular energy is expended producing heat and in active transport of ions and water.

In some embodiments, the sensors of the device for generalized disease learning are selected based on the following estimations of energy budget: (1) At a physiologic scale, the total energy expenditure is the sum of resting energy, diet energy and physical energy: Total Energy Expenditure (EE)=Resting EE+Physical Activity EE+Diet EE. This is a physiologic energy budget; (2) The resting energy expenditure is roughly 80% of total energy expenditure: REE˜80% EE; (3) Humans are inefficient machines in that ˜60% of energy is lost as heat and only 40% captured as work: EE˜60% ΔQ+40% ΔW; (4) The work done within cells is largely that of moving water and ions, synthesizing proteins and biochemical (intermediary metabolism). Therefore ΔW is estimated at 25% ion movement; 25% structure; 50% biochemical; (5) The heat produced within cells is the same heat produced by organs and the same heat detected leaving the body through the skin. Therefore ΔQ is the same; (6) Ideally a device would quantify all work done, however the V1.0 device focuses on sensors that were largely commercially available; and (7) As there are sensors commercially available that allow quantifying heat flux and work associated with water, the accuracy of the Device #1 is estimated to be at ˜85% of the total ΔE or Energy Expenditure. This accuracy is sufficient to quantify an energy budget and capture work done involving water* to quantify and optimize health and pre-symptomatically detect and intercept disease.

In some embodiments, attributes of the device for generalized disease learning include:
1. Battery Life: The battery life of the device is at least 180 days, batteries are disposable, and internally powered by a standard 3-Volt (peak recharge) lithium coin cell battery of 50 mAh total capacity. This allows for continuous measurement over long period of time;
2. Usability: The device is rugged, inexpensive, and simple to use which allows for widespread deployment in hospital, assisted care, and ambulatory medical settings;
3. Software: The device readily interfaces to the IoT (cloud/machine learning) which allows for the automation of data recording and analysis;
4. Simple: The device requires no specialized skills to operate. This allows for widespread deployment of a decentralized health measurement system;
5. Inexpensive: A commercial cost of <$100.00 is estimated. This lowers financial hurdles to broad deployment;
6. Automated: The device measures your health continuously which allows for early detection of change before disease symptoms and allows for the generation of big data sets to detect and learn during population-based health threats;
7. Rugged Design Standards: The device is designed to operate reliably in a harsh environment and/or harsh conditions. This allows for deployment in diverse civilian, first-responder, and warfighter settings;
8. Secure: All communication between the device and smartphone are done over an encrypted Bluetooth Low Energy link, specifically “LESC”. No identity information about the user is stored on the wrist sensor or transmitted unencrypted by Bluetooth. The connection between the app and cloud storage servers is secure. At each stage the data is encrypted at rest (on device or cloud) and during transmission. Protected Health Information will be gathered and moved to secure cloud only with consent of the device wearer. Access to data in cloud for purposes of analytics development will reveal only formally de-identified data;
9. Safe: The sensor module is an external skin surface applied battery powered sensor suite contained within an isolated polymer (Delrin) encasement with only non-electrically conductive surface materials of exposure. There are no electrical contacts between the internal electrical circuitry and the skin.

In some embodiments, the platform for generalized disease learning is equipped with a learning engine, mobile application software, a cloud infrastructure capable of continuously ingesting data from our proprietary sensor hardware for the purpose of description, prediction, and inference regarding disease. The ingested data will include energy measurements and auxiliary sensor data (biological or environmental) which are intended to characterize metabolic tasks and other biological processes to aid in the quantification of energy expenditures and signatures. The learning engine will execute the following functions:
Quantify energy signatures;
Correlate energy signatures with function, disease, outcome;
Infer the rules of an energy budget through relationship of annotation and energy signature;
Correlate energy budget with function, disease, outcome;
Refine and test energy budget quantification via app-based recommendation and feedback;
Infer the health capacity associated with an energy budget under various stresses as indicated by the probability of persistence;
Identify the critical regularities within energy budgets which are necessary and sufficient conditions for Health Capacity: the rules of Health Capacity;
Identification of ‘energy gaps’ as evidence of the need for an additional annotation or sensor.

### Example 2

### System for Closed Loop Control of Thermoregulation, Metabolism, Weight, or Like Property

### Operating Principle of the System for Closed Loop Control of a Property

In one aspect, the disclosed technology uses heat and physiological exhaust signals to inform a closed loop control system over an organism. In closed loop control, or feedback control, the control action from the controller is dependent on feedback from the process in the form of the value of the process variable.

The internal energy management of an organism (biology or synthetic) is critical to its continued function. This energy constraint (tied to the first law of thermodynamics, the conservation of energy) plays out in diverse ways in various species and systems but is always present and central. Energy which is controlled and spent by an organism will necessarily re-emerge as exhaust—a material emission which has a lower Gibbs free energy density than the fuel which powers the organism. The difference in free energy between fuel and exhaust can be exactly accounted for by work performed by the organism, internal or external.

For every organized system, the exhaust streams (both heat and matter) have two components. The first component is understood colloquially as arising from “inefficiencies.” Machines generate waste heat which is related in some sense to the work they perform. Imperfections, sub-optimal circumstances, and intrinsic limitations will also limit the complete conversion of fuel energy into useful work. Therefore, for every unit of work performed, a trace of wasted energy can be detected with a significantly similar temporal pattern to the work output. The second component of exhaust arises not from inefficiencies associated with the performance of work but from the finite and on-going cost of remaining functionally embodied. This component of exhaust is less well understood and has no colloquial term. In biological systems it is referred to as a basal metabolic rate.

The distinction between the two components of exhaust is fuzzy at the margin. Nonetheless, there are practical reasons to work toward a discrete delineation between the two. Consider for example a car which is reported to be highly fuel efficient, say 75 mph while in motion, but has very inefficient and dirty combustion while idling. An appreciation for both attributes is essential for understanding the possible function of such a vehicle; it is better suited for long-distance highway travel than for delivering the mail. Furthermore, the distinction is useful in understanding the most valuable ways in which the car can be improved. If such a vehicle were used for mail delivery, any small optimization to the idle efficiency would have much higher impact than an improvement to the fuel efficiency while in motion.

In biology, the term “decoupling” is often used to describe the separation between energy expenditure and work. This term is typically understood as a specific reference to a set of five transporter enzymes which live on the inner mitochondrial membrane, called uncoupling proteins. These enzymes uncouple the proton gradient at the inner mitochondrial membrane from the synthesis of ATP. However, there are other uncoupled systems in biology. For instance, ATP powers the maintenance of ionic gradients in the body which have their own regulation and decoupling from work. Without being bound by the exact mechanism of such an decoupling, heat is generated and these functions are regulated. Such gradients occur in muscle, and throughout the nervous system. Further, the heat generated is used in thermoregulation, so it is not correct to call it a mere “inefficiency.” To understand the function of these critical tissues and an entire organism, their total energy expenditure, in active and/or inactive states, may be used to inform the learning algorithm.

The central importance of decoupling proteins and signaling around metabolic regulation suggest the critical importance of information in organism health. This is recapitulated externally in the development of digital health wearables which purport to feedback information and analysis to an individual such that they can make better decisions about their health. However, the current market of wearables typically measures proxies of work: steps, activity, heart rate elevation. While these measurements have value, they do not capture a full picture of energy expenditure because they neglect the basal component. This limits their usefulness in a variety of use cases related to health, disease and performance.

In some embodiments, the disclosed technology relates to a system which measures exhaust, embodied primarily as heat but including other low energy chemical forms, for the purpose of understanding, improving, modulating, or repurposing the function of an existing organism. In the case of human health, one embodiment comprising a heat sensor enables individuals to manage their health not through measurements of activity and work and the typical estimates of marginal energy expenditure, but through a direct measure of total energy expenditure in real time. Such system may be particularly valuable in the management of weight, blood pressure, and circadian rhythm, all of which have significant functional intersection with thermoregulation.

The control system takes information from measured exhaust streams (which are emergent properties of the organism), performs an automated analysis on the exhaust information streams, and may then influence the organism through three distinct channels: 1) Automatic, direct treatment by a (de)-coupling agent, 2) automated control of the external environment, 3) automated recommendation to the individual to take some action.

### 1) Automatic Treatment by a (De)-Coupling Agents

An insulin pump measures glucose to determine an appropriate dose of insulin, which is administered in an automated manner, enabling a closed loop control system about blood sugar. Similarly this first mode of operation measures heat or exhaust to automate dosing and administration of a decoupling agent or other modulator of oxidative phosphorylation or other ionic gradient structure where stored electrochemical energy may be decoupled from pathways which execute some physiologic work or function.

### 2) Automated Control of the External Environment

In this mode, an external physical parameter such as an interior air temperature, pressure, or humidity, may be used to influence thermoregulatory function or other physiology which is related to thermoregulation, including cardiovascular parameters, circadian parameters, cognitive and emotional parameters. A diverse set of external parameters may be effective for an individual including not just ambient physical conditions typically called “climate control” but also highly specific information dense experiences such as music, familiar smells, visual art or other stimuli. Again, the feedback variable of heat or other exhaust will be used to dose these external treatments.

### 3) Automated Recommendation to the Individual to Take Some Action

This mode describes a category of interactions which cannot be automated because they involve volitional activity of a human in the loop. A heat signature analysis may suggest that an individual, change their clothes, go inside/outside, eat a specific food, drink water, do some exercise, etc. This type of control system is not fully closed because the treatment cannot be fully automated. Instead, an automated recommendation or ‘call to action’ message will be sent in such a way that timely change may be taken (e.g. mobile SMS or other messaging). The closed loop control becomes complete through the interaction and cooperation of the human in the loop.

### Feedback Control

Control theory may be used to inform system-control-design principles and to produce controllers with the flexibility and intelligence to control systems to select a current operational mode from among different possible operational modes and then provide control outputs to drive the controlled system to produce the selected mode of operation. Various different types of controllers are commonly employed in many different application domains, from simple closed-loop feedback controllers to complex, adaptive, state-space and differential-equations-based processor-controlled control systems. In some embodiments, controllers are designed to output control signals to various dynamical components of a system based on a control model and sensor feedback from the system. In some embodiments, systems are designed to exhibit a predetermined behavior or mode of operation, and the control components of such systems are therefore designed, by design and optimization techniques, to ensure that the predetermined system behavior transpires under normal operational conditions. In certain cases, there may be various different modes of operation for a system, and the control components of the system therefore need to select a current mode of operation for the system and control the system to conform to the selected mode of operation.

In some embodiments, the disclosed technology uses a general class of intelligent controllers that determine the presence and absence of one or more types of entities within one or more areas, volumes, or environments affected by one or more systems controlled by the intelligent controllers and that includes many different specific types of intelligent controllers that can be applied to, and incorporated within, many different types of devices, machines, systems, and organizations. Intelligent controllers control the operation of devices, machines, systems, and organizations that, in turn, operate to affect any of various parameters within one or more areas, volumes, or environments. The general class of intelligent controllers to which the current application is directed include components that allow the intelligent controllers to directly sense the presence and/or absence of one or more entities using one or more outputs from one or more sensors, to infer the presence and/or absence of the one or more entities within areas, regions, volumes or at points within areas, regions, and volumes from the sensor-based determinations as well as various types of electronically stored data, rules, and parameters, and to adjust control schedules, based on the inferences related to the presence or absence of the one or more entities within the areas, regions, and volumes.

In some embodiments, the disclosed technology uses smart devices, including one or more different types of sensors, one or more controllers and/or actuators, and one or more communications interfaces that connect the smart devices to other smart devices, routers, bridges, and hubs within a local smart environment, various different types of local computer systems, and to the Internet, through which a smart device may communicate with cloud-computing servers and other remote computing systems. Data communications may be carried out using any of a large variety of different types of communications media and protocols, including wireless protocols, such as Wi-Fi, ZigBee, 6LoWPAN, various types of wired protocols, including CAT6 Ethernet, HomePlug, and other such wired protocols, and various other types of communications protocols and technologies. Smart devices may themselves operate as intermediate communications devices, such as repeaters, for other smart devices.

Smart devices within a smart environment can communicate through the Internet via 3G/4G wireless communications, through a hubbed network, or by other communications interfaces and protocols. Many different types of data can be stored in, and retrieved from, a remote system, including a cloud-based remote system. The remote system may include various types of statistics, inference, and indexing engines for data processing and derivation of additional information and rules related to the smart environment. The stored data can be exposed, via one or more communications media and protocols, in part or in whole, to various remote systems and organizations. External entities may collect, process, and expose information collected by smart devices within a smart environment, may process the information to produce various types of derived results which may be communicated to, and shared with, other remote entities, and may participate in monitoring and control of smart devices within the smart environment as well as monitoring and control of the smart environment. In some embodiments, export of information from within the smart environment to remote entities may be strictly controlled and constrained, using encryption, access rights, authentication, and other techniques, to ensure that information deemed confidential by the smart manager and/or by the remote data-processing system is not intentionally or unintentionally made available to additional external computing facilities, entities, organizations, and individuals.

Various processing engines within the external data-processing system can process data with respect to a variety of different goals, including provision of managed services, various types of advertising and communications, social-networking exchanges and other electronic social communications, and for various types of monitoring and rule-generation activities. Various processing engines communicate directly or indirectly with smart devices, each of which may have data-consumer (“DC”), data-source (“DS”), services-consumer (“SC”), and services-source (“SS”) characteristics. In addition, the processing engines may access various other types of external information, including information obtained through the Internet, various remote information sources, and even remote sensor, audio, and video feeds and sources.

In some embodiments, an intelligent controller controls a device, machine, system, or organization via any of various different types of output control signals and receives information about the controlled entity and an environment from sensor output received by the intelligent controller from sensors embedded within the controlled entity, the intelligent controller, or in the environment. The intelligent controller may be connected to the controlled entity via a wire or fiber-based communications medium. Alternatively, the intelligent controller may be interconnected with the controlled entity by alternative types of communications media and communications protocols, including wireless communications. In some embodiments, the intelligent controller and controlled entity may be implemented and packaged together as a single system that includes both the intelligent controller and a machine, device, system, or organization controlled by the intelligent controller. The controlled entity may include multiple devices, machines, system, or organizations and the intelligent controller may itself be distributed among multiple components and discrete devices and systems. In addition to outputting control signals to controlled entities and receiving sensor input, the intelligent controller also provides a user interface through which a human user can input immediate-control inputs to the intelligent controller as well as create and modify the various types of control schedules, and may also provide the immediate-control and schedule interfaces to remote entities, including a user-operated processing device or a remote automated control system. In some embodiments, the intelligent controller provides a graphical-display component that displays a control schedule and includes one or more input components that provide a user interface for input of immediate-control directives to the intelligent controller for controlling the controlled entity or entities and input of scheduling-interface commands that control display of one or more control schedules, creation of control schedules, and modification of control schedules.

In other embodiments, intelligent controllers may receive sensor input, output control signals to one or more controlled entities, and provide a user interface that allows users to input immediate-control command inputs to the intelligent controller for translation by the intelligent controller into output control signals as well as to create and modify one or more control schedules that specify desired controlled-entity operational behavior over one or more time periods. The user interface may be included within the intelligent controller as input and display devices, may be provided through remote devices, including mobile phones, or may be provided both through controller-resident components as well as through remote devices. These basic functionalities and features of the general class of intelligent controllers provide a basis upon which automated control-schedule learning, to which the current application is directed, can be implemented.

In some embodiments, an intelligent controller may be implemented using one or more processors, electronic memory, and various types of microcontrollers, including a microcontroller and transceiver that together implement a communications port that allows the intelligent controller to exchange data and commands with one or more entities controlled by the intelligent controller, with other intelligent controllers, and with various remote computing facilities, including cloud-computing facilities through cloud-computing servers. In some embodiments, an intelligent controller includes multiple different communications ports and interfaces for communicating by various different protocols through different types of communications media. In some embodiments, intelligent controllers use wireless communications to communicate with other wireless-enabled intelligent controllers within an environment and with mobile-communications carriers as well as any of various wired communications protocols and media. In certain cases, an intelligent controller may use only a single type of communications protocol, particularly when packaged together with the controlled entities as a single system. Electronic memories within an intelligent controller may include both volatile and non-volatile memories, with low-latency, high-speed volatile memories facilitating execution of control routines by the one or more processors and slower, non-volatile memories storing control routines and data that need to survive power-on/power-off cycles. Certain types of intelligent controllers may additionally include mass-storage devices.

In some embodiments, an intelligent controller includes controller logic implemented as electronic circuitry and processor-based computational components controlled by computer instructions stored in physical data-storage components, including various types of electronic memory and/or mass-storage devices. Computer instructions stored in physical data-storage devices and executed within processors comprise the control components of a wide variety of modern devices, machines, and systems, and are as tangible, physical, and real as any other component of a device, machine, or system. The controller logic accesses and uses a variety of different types of stored information and inputs in order to generate output control signals that control the operational behavior of one or more controlled entities. The information used by the controller logic may include one or more stored control schedules, received output from one or more sensors, immediate control inputs received through an immediate-control interface, and data, commands, and other information received from remote data-processing systems, including cloud-based data-processing systems. In some embodiments, in addition to generating control output, the controller logic provides an interface that allows users to create and modify control schedules and may also output data and information to remote entities, other intelligent controllers, and to users through an information-output interface.

In some embodiments, an intelligent controller receives control inputs from users or other entities and uses the control inputs, along with stored control schedules and other information, to generate output control signals that control operation of one or more controlled entities. Operation of the controlled entities may alter an environment within which sensors are embedded. The sensors return sensor output, or feedback, to the intelligent controller. Based on this feedback, the intelligent controller modifies the output control signals in order to achieve a specified goal or goals for controlled-system operation. In essence, an intelligent controller modifies the output control signals according to two different feedback loops. The first, most direct feedback loop includes output from sensors that the controller can use to determine subsequent output control signals or control-output modification in order to achieve the desired goal for controlled-system operation. In some cases, a second feedback loop involves environmental or other feedback to users which, in turn, elicits subsequent user control and scheduling inputs to the intelligent controller. In other words, users can either be viewed as another type of sensor that outputs immediate-control directives and control-schedule changes, rather than raw sensor output, or can be viewed as a component of a higher-level feedback loop.

In some embodiments, an intelligent controller continuously operates within the context of an event handler or event loop. To start, the intelligent controller waits for a next control event. When the next control event occurs, then, in a series of conditional statements, the intelligent controller determines the type of event and invokes a corresponding control routine. In the case of an immediate-control event, the intelligent controller calls an immediate-control routine, to carry out the intelligent controller's portion of a user interaction to receive one or more immediate-control inputs that direct the intelligent controller to issue control signals, adjust a control schedule, and/or carry out other activities specified by a user through an intermediate-control interface. In the case that the control event is a scheduled control event, such as when the current time corresponds to a time at which a control schedule specifies a control activity to be undertaken, then a schedule-control routine is called, to carry out the scheduled control event. When the control event is a schedule-interface event, then the intelligent controller invokes a schedule-interaction routine, to carry out the intelligent controller's portion of a schedule-input or schedule-change dialog with the user through a schedule interface. In the case that the control event is a sensor event, then a sensor routine is called by the intelligent controller to process the sensor event. Sensor events may include interrupts generated by a sensor as a result of a change in sensor output, expiration of timers set to awaken the intelligent controller to process sensor data of a next-scheduled sensor-data-processing interval, and other such types of events. When the event is a presence event, then the intelligent controller calls a presence routine. A presence event is generally a timer expiration, interrupt, or other such event that informs the intelligent controller that it is time to determine a next current probability-presence scalar value or to construct a next current probability-presence map. Many additional types of control events may occur and may be handled by an intelligent controller, including various types of error events, communications events, power-on and power-off events, and a variety of events generated by internal set components of the intelligent controller. There are many different models that describe how various different intelligent controllers respond to detected presence and/or absence of human beings. As discussed above, during intelligent-controller operation, the intelligent controller continuously evaluates a wide variety of different types of electronically stored data and input data to update stored indication of the probability of human presence in each of one or more regions within an environment controlled by the intelligent controller. In one model, the intelligent controller primarily operates with respect to two different states: (1) a presence state resulting from determination, by the intelligent controller, that one or more human beings are present within one or more regions; and (2) a no-presence state, in which the intelligent controller has determined that no human beings are present within the one or more regions.

In a scheduled-control event, the intelligent controller receives an indication of the scheduled control that may be considered to be carried out by the intelligent controller. A scheduled control may be either a scheduled setpoint or may be a preconditioning time point in advance of a scheduled setpoint at which the intelligent controller would begin actively adjusting an environmental parameter in order that the environmental parameter reach the desired value at the time of the scheduled setpoint. When the intelligent controller is in the long-term no-presence state, the routine “scheduled control” returns, since scheduled setpoints and preconditioning points are ignored in the long-term no-presence state. When the intelligent controller is in the no-presence state, the intelligent controller calls the routine “evaluate setpoint corresponding to scheduled control” to determine whether or not to carry out the setpoint. When the routine returns a TRUE value, then the intelligent controller transitions to the temporary-assumed-presence state and the scheduled control is carried out. Otherwise, when the routine returns a FALSE value, the routine “scheduled control”. Note that the scheduled control is carried out when the intelligent controller is in the presence state.

In alternative embodiments, various different implementations of an intelligent controller that selectively carries out scheduled control operations during a period when an entity is not detected in a controlled environment can be obtained by varying any of many different design and implementation parameters, including intelligent-controller hardware, operating systems and other control programs used in the intelligent controller, and various implementation parameters for controller functionality, including programming language, modular organization, data structures, control structures, and other such parameters. Various different considerations may be applied to determine the range of times during which the absence of an entity's presence may result in a transition to the no-presence or long-term no-presence states. The range of times, for example, may be determined from accumulated sensor and/or probability-of-presence data. Similarly, various different models and computations may be employed to determine the variable threshold amount of time to wait following carrying out a scheduled control, in the temporary-assumed-presence state before reverting to energy-efficient settings and transitioning back to the no-presence state. Additionally, many different types of methods and considerations may be employed to evaluate a scheduled control operation during times when an entity, such as a human being, is not considered to be present in the controlled environment.

In some embodiments, the disclosed technology incorporates closed-loop identification, a technique where process model parameters are identified based on data from a process operating under closed-loop control. It is often desirable to be able to update or replace a process model based on closed-loop data since this can eliminate the need to turn off automatic controls and disturb the process to generate open-loop data. However, one problem with closed-loop identification is that using standard identification techniques (such as those suitable for analysis of open-loop data) can result in biased or inaccurate model parameter estimates, particularly when using direct identification methods without any knowledge about true process and noise model structures. In identifying process model parameters, the disclosed system may use closed-loop process data while reducing or avoiding bias in the identified model parameters. These techniques enable automatic closed-loop process model updating to be used for model-based controllers. These types of techniques can provide various benefits depending on the implementation. For example, the proposed techniques can overcome biasing when performing closed-loop identification, resulting in more accurate process models being generated. Moreover, with automatic closed-loop process model updating, model-based controls could be maintained to perform at the highest level without needing to take those controls offline for plant experiments. Further, these approaches can reduce the time and effort needed in updating process models. In addition, overall controls can be kept functioning at high level at all times, reducing losses due to poor quality production.

In one approach, closed-loop data associated with a model-based process controller is obtained. This could include, for example, a processing device collecting the data during execution of control logic by the process controller. The collected data can include routine operating data that is generated as the process controller executes its control logic and attempts to control at least one industrial process (or portion thereof), such as controlled variable measurements and manipulated variable adjustments. Then, the closed-loop data is analyzed to identify at least one disturbance model. This could include, for example, the processing device performing a model identification algorithm using the data. In particular embodiments, the processing device implements a high-order autoregressive with exogenous terms (ARX) model identification algorithm that can fully capture the noise model dynamics without needing information about the true noise model. Part of the high-order ARX model identification algorithm can include identifying a noise model associated with noise related to the industrial process. Then, the closed-loop data is filtered using an inverse of the disturbance model. This could include, for example, the processing device using an inverse of the previously-identified disturbance model to filter the closed-loop data. Then, model parameter estimates for a process model are estimated using the filtered closed-loop data. This could include, for example, the processing device performing a model identification algorithm using the filtered data, such as an output-error (OE) model identification algorithm or other model identification algorithm. The model parameters are used in some manner. This could include, for example, the processing device generating a new process model or updating an existing process model and providing the new or updated process model to a process controller. This could further include the processing device updating the process model used by control logic of the process controller.

### Example 3

### Applying “Thermal Fit” Model to Improve Health

In some aspects, the health of an organism-its ability to adapt and to persist—is an emergent property which can be quantified, not simply in a post-hoc manner (e.g., “Survival frequency of phenotype A is higher than phenotype B”) but as a direct physical measure of the organism. This physical measurement may be predictive of functional or health outcomes, including survival, and can be used to enable and justify interventions which improve the odds of positive outcomes.

### Outline of a Model Based on “Thermal Fit”

In some embodiments, the disclosed technology applies a “Thermal fit” model to improve health and health outcomes. In some embodiments, the disclosed technology measures, and learns, the surface heat flux of a biological system as a function of circadian rhythm, and may use such measurements and learning to infer or derive indicators of the health state of the biological system. The disclosed technology may further recommend actions to be taken to improve health, or health outcomes, of the biological system based on the indicators.

Certain premises of the “thermal fit” model include:
1. Circadian rhythm may be an anticipatory system.
2. When a body sensory apparatus does not match the expectations of the circadian rhythm, adrenergic tone may increase.
3. Heightened adrenergic tone may aid focus, learning, or other adaptive techniques over short periods of time.
4. In some cases, the longer a biological system, e.g., a person, lives, the more it experiences, and the more likely it is that its expectations of the circadian rhythm will become mutually unsatisfiable.
5. In some cases, if a biological system has a set of mutually unsatisfiable expectations, adrenergic tone may become chronically elevated.
6. Chronic elevations of adrenergic tone may be associated with inflammatory disorders and/or frailty.

Certain predictions derived from the “t” model include:
1. In some cases, the health risks of being a night owl only exist if a person becomes a night owl later in adulthood. In some cases, lifelong night owl status creates no circadian confusion or health risk.
2. In some cases, it may be possible to “forget” or otherwise unlearn metabolic experience to rollback unsatisfiable circadian expectations.
   a. In some cases, the circadian memory may be stored in the dynamic clock system.
   b. A forgetting mechanism may involve mathematics described by phase transitions in the Kuramoto model.
   c. Entrainment by Colored light therapies may be able to “erase” expectations from the clock systems.
3. In some cases, long-lived blue zoners may have the most stable circadian rhythms of all, with low fragmentation.
4 Circadian confusion from unsatisfiable expectations may be a primary cause of depression
5. Circadian clocks may have a natural speed which is biased to be longer than 24 hours.

### Background Applicable to Application of a “Thermal Fit” Model

All of life, as we know it, depends on chemistry. A set of interrelated chemical reactions are carried out that are called, collectively, metabolism. There are those chemical reactions that degrade substances—catabolism—and those that assemble substances-anabolism. The majority of these chemical reactions may be carried out by mechanisms of biological catalysts, e.g., enzymes. Irrespective of whether these reactions are originally carried out by prosthetic groups, metals or other reaction centers, the rate of a chemical reaction may have a dependency on temperature as defined by the Arrhenius equation. In some cases, for life to have occurred, it would have to had developed a “way” to control internal temperature, for if it had not, the rate of chemical reactions (metabolism) would have varied so significantly as to not enable sustainable and reproducible function. Hence, the regulation of temperature may be inseparable from life. This is in part why organism have been classically divided into exotherms and endotherms, as how they manage temperature so significantly relates to function. While there may be thousands of ways this is done by flora and fauna, they all achieve essentially the same result: a reproducible temperature to carry out the chemistry of an organism that enables persistence and adaptation. Especially when starting with the first single cell organism or protocell. Hence, for life to occur, persist, and adapt, biological systems may have had to “control” temperature.

How does a biological system control temperature? While there may be many variables that affect the temperature inside of an organism, there is one that may predominate—the external temperature. As between two organisms, one that could “sense” ambient temperature and respond, and another that could not, the former would more likely survive. As temperature is an overarching parameter that may affect metabolic rate (and the chemistry of life's processes), the organism that can best sense and respond to ambient temperature may win. In some cases, then, organisms have emergently become capable of managing and/or controlling temperature using unique temperature management strategies/apparatus. In some cases, what is called circadian rhythm is at least one method an organism may employ to manage heat to achieve the right temperature to function and adapt/persist under a set of external conditions. In some cases, an optimal circadian rhythm is one that results in optimal health capacity—the ability to carry out metabolism at a prescribed rate which is highly dependent upon temperature to adapt and persist. Hence, organisms that possess an ability to anticipate and synchronize to the environment to off-load heat in a controlled manner to optimize their metabolic fate may win. The anticipation and synchronization may be what Spencer referred to as “fit”-building on Darwin's concepts, and may be the local and immediate access to resources, critical amongst them ambient temperature. In some cases, as the single molecule which shepherds the majority of metabolic heat is water, water may be (largely) responsible for “fitness.” In some embodiments, an aspect of the disclosed technology measuring the heat transfer properties of water. This would track and allow deciphering of (1) molecular basis of “fit-ness”, (2) life; (3) changes in fitness precede the onset of failure (disease), early detection of disease; and (4) using heat elimination to portend better function, a first measurable parameter of health-“thermal fit.”

Thermal fit is akin to skipping rope. For example, one is skipping rope and there is a frequency and magnitude that one uses to skip that matches by body's rhythm—the strength, height and energy. However, one doesn't jump rope in isolation, one is jumping in the “real world,” for example on a tread mill that can change grade, rate or perhaps deformation of a landing mat. Depending on the external conditions, one needs to change the jumping technique to harmonize with the external forces that have their own frequencies-perhaps speed up going downhill, slow down going up-hill, push harder when on sand or other loose surface, or push less hard when on concrete or other firm surface.

In some aspects, the disclosed technology relates to the following concepts:
1. Emergence
2. Genotype+Environment=Phenotype
3. Darwin/Spencer Fit
4. Temperature
5. Heat
6. Thermal fit
7 Circadian rhythm
8. Homeostasis
9. Anomaly detection/prediction
10. Health capacity

In some embodiments, these concepts can be used to construct a Venn diagram. In some embodiments, the central overlap of this Venn diagram indicates the disclosed technology.

### Further, Alternative Details of a “Thermal Fit” Model

In some embodiments, the disclosed technology relates to the following aspects:
1. Living organisms are complex chemical systems whose function is highly dependent upon internal temperature.
2. Human function may be described by genotype+environment=phenotype.
3. This general interrelationship between an organism and its environment may be described by Darwin/Spencer as fit—an ability to efficiently extract resources from the “local and immediate environment.”
4. As used herein, “thermal fit” relates to one particularly important aspect of “fit” being thermal—the difference between the interior temperature of the organism and the temperature of the environment results in a flow of heat.
5. While “fit” is a multi-faceted concept which may be generally difficult to describe comprehensively, thermal fit may involve known thermal physics which are largely interpretable, measurable, and quantifiable.
6. The primary thermal cycle to which all life must fit itself may be the day-night cycle which creates consequential swings in ambient temperature. In some cases, the primary—or initial—role of clock (circadian) proteins is to optimize an organism's thermal fit across the diurnal temperature cycle to sustain function. Specifically, to match “the right heat generation pattern with the right ambient temperature pattern.” In doing so, unfavorable fluctuations in core temperature (which significantly impact rates of chemical reactions of core metabolism) are avoided, enabling greater chemical/metabolic efficiency (function) which enables adaptation and persistence.
7. Through the prism of thermal fit, the Darwinian driver of living systems may be to most efficiently extract free energy from the environment and to do this while minimizing heat loss such that they can maximize functional growth of biomass (maturation or reproduction), and to do so with minimal unfavorable fluctuations in core temperature which are detrimental to both health and function.
8. The metabolic stability of healthy organisms-baseline/normal homeostasis—may be a result of a thermal fit which emerged from an evolutionary selection process.
9. In some cases, thermal fit may be the primary organizing principle (that rightly sits at the nexus of nature/nurture, genome/environment) of biology and that circadian rhythm describes its manifestation over time, through our prism circadian rhythm is thermal fit.
10. By measuring the flow of heat from an organism, the disclosed technology may have a direct representation of its (unique) circadian rhythm.
11. By analyzing changes in heat flow patterns, the disclosed technology can detect anomalies, e.g., disease.
12. By analyzing changes in heat flow patterns as a function of eating, sleeping and exercise, all of which are primary variables of heat generation or elimination, the disclosed technology can improve thermal fit and health.
13. By learning the relationship between thermal fit and function, the disclosed technology can learn the rules of homeostasis.
14. Because the chemical reactions that constitute metabolism and support homeostasis in all living systems may have consequential sensitivity to temperature over the range seen in the day/night cycle, the “first hurdle” life might have to overcome was the management of its internal temperature. Hence, there is an inextricable link between the control of temperature and function. Those organisms that control temperature the best may be the fittest and adapt and persist, with more efficient energy use. Those that don't and experience wide fluctuations which are ill tolerated, may not adapt and may perish.

In some embodiments, the disclosed technology further relates to the following aspects:
1. Biological systems are in part emergent systems, not engineered. No matter how hard you look, you may not find life or its function in a single chemical.
2. The rate of chemical reactions may be proportional to temperature in some cases.
3. The survival of the fittest may not be about going to the gym.
4. Nature versus nurture.
5. The earth rotates and its temperature changes.
6. The first and second laws of physics in part apply to biology.
7. Changes in heat elimination predict illness.

As an example, one's body has sensory functions which detect the slope and speed of the treadmill. But if one always relies on the senses one may be always alert-chronically high adrenergic tone. Instead, one's body learned the diurnal pattern of the treadmill and then one mostly knows what to anticipate. One can relax and allow the internal clocks to predict and navigate through the daily changes in treadmill speed. This allows biological systems to operate in a less stressed mode than is required by constant sensing. By recognizing the pattern of work and being prepared to deal with it, it becomes less stressful

In some cases, if there is a sudden change in treadmill speed which does not match the daily pattern, one's body may have a mechanism to identify the deviation from expectation, and to respond by cranking up sensory acuteness, adrenergic tone, etc. This is a great system which enables the low-stress pattern-following behavior to get one through the predictable parts of life but still allows one have the capacity for rapid responsiveness to everything that is unpredictable. In some cases, this also facilitates learning about rare dangerous events. This dual system of adapted-ness corresponds to one's own experience of life. In some cases, it functions at a cellular level through the circadian clock genes.

In some embodiments, the disclosed technology addresses confusion or un-satisfiability. As an example, what happens if the body's clock anticipates two contradictory states at once, e.g., the body simultaneously expects the treadmill to be both fast and slow. At least one of these two expectation will always be wrong. In some cases, biological systems may look healthy and can still physically navigate the treadmill by the senses, but they may have lost a critical physiologic function. For example, they can no longer drop into the low key states which allows circadian rhythm to direct their steps. In some cases, the mutual un-satisfiability of the two expectations means that the biological systems are chronically under stress, living with high adrenergic tone, and navigating by senses which are imperfect, and prone to error when fatigued. In some cases, the accumulation of mutually unsatisfiable expectations over time leads to chronic stress and eventual frailty. In some cases, this constitutes a new theory of aging which may lead to novel therapies.

In some embodiments, the disclosed technology relates to mining the data. In some embodiments, a technological model will be looking for high-dimensional relationships between something complex and some value outcome. In some embodiments, this complexity relates to the number of parameters required to describe an adequate predictive model. In some embodiments, the disclosed technology relates to deep learning systems having millions or billions of parameters. In some embodiments, to train a model with a billion parameters will require billions of training examples.

In some embodiments, the disclosed technology looks for disruptions in circadian rhythm. There may be two elements that shorten the learning curve effectively. First, the low-dimensionality. Healthy physiology includes a stable circadian rhythm which can be described (well enough) by only 6 parameters. With just a handful of individuals who have been measured by the disclosed device, differences in these parameters can be determined. For example, as few as 20 participants may be required. Second, starting from physical reasoning, the disclosed technology may have strong expectations of how these 6 parameters will change in many cases. Therefore, the disclosed technology may only need to test hypotheses by simple statistics.

For example, based on purely physical terms, a patient approaching a critical phase of congestive heart failure may have the following shifts in the 6 circadian parameters:
1. Downward shift in the power spectrum of heat fluctuations
2. Decrease in amplitude
3. Decrease in relative amplitude
4. Delay in circadian phase
5. Decrease in inter-day stability
6. Increase in Intraday Variability

In some cases, a primary aspect of thermoregulation which is under central control by the hypothalamus, is the vasoconstriction or dilation of peripheral microvasculature which in many cases accounts for the largest and most controllable component of physiologic heat loss. By contrast, truncal heat loss has a less agile control system and is limited by the surface-to-volume ratio intrinsic to its geometry and the anatomical structure which limits blood flow near the surface of a biological system.

Exemplary Device Applying and/or Utilizing the “Thermal Fit” Model

In some cases, joints adjacent to the trunk offer a third option for heat loss. Blood flow in these regions (neck, armpit, groin) has several aspects which make it useful as a locus of heat loss: near the surface, high volume, consistent flow of blood. Whereas peripheral vascular beds are highly controlled and blood flow can be largely shut down, the trunk-adjacent joints have medium sized conduits for blood which are relatively constant in flow even when the hypothalamus has vasoconstricted the periphery. Therefore, these sites may exhibit ideal properties for external control. Indeed, they are already used as secondary heat loss sites under certain vasoconstrictions stress situations. For example, a runner may be vasoconstricted after a short sprint and may put arms up to allow heat loss from armpits, or squat to open up heat loss from the groin.

In some embodiment, the disclosed technology relates to the use of a smart garment that aids in the passive or active control of thermoregulation by placing wirelessly controlled thermal elements at the trunk-adjacent joints. The specific embodiments may depend very much on the types of materials which can be designed. Each of these locations may require unobstructed movement so bulky thermoelectric units may not be practical if placed directly at the site.

In some embodiments, a disclosed measurement and healing device may include or relate to the following aspects:
1. Primarily cooling, but also therapeutic heating of the entwined arteries and veins at the trunk-adjacent joints (neck, armpit, groin).
2. In some cases, thermal elements may not obstruct movement. However, this may be less important if the user is immobile (from paralysis, for example).
3. Active elements, (e.g., Peltier effect cooler) may be miniaturized and made flexible and embedded at the site of the joint or placed remote from the joint (mid-back) and serve coolant (air or fluid) to the joint area by means of conduits which are integrated into the garment.
4. The rate of cooling/heating may be controlled by a central control unit which may be in hardware on the garment, in a mobile app, or in the cloud, with information relayed back to the garment through a mobile device.
5. The garment may include temperature sensitive elements at the joints which enable effective control.
6. Auxiliary sensors such as the “Enerji band” may detect the peripheral heat flux as a ground truth measure of the heating cooling effect, which may enable more exquisite control.
7. In some cases, control timescales may be on the scale of minutes, seconds, or milliseconds, so connectivity may be continuous and digital, not controlled manually.

### Example Use Cases of Exemplary Device Applying the “Thermal Fit” Model

In some cases, the disclosed device may be used for control in diabetics. Diabetics are limited in their ability to loss heat in a variety of ways. For this group, an active cooling effect may be helpful and prudent. Therefore, the control logic in this case may be to do as much cooling as is comfortable for the user. For example, based on calculation of the learned model, the device can shift a diabetic's energy balance by certain kCals per day if they wear a cooling garment for certain hours per week.

In some cases, the disclosed device may be used for augmentation of thermoregulation in healthy individuals. Thermoregulation in healthy individuals is complex and adaptive. For example, there are times where physiologic thermoregulation could be usefully augmented: 1) aid in heat loss during intense exercise; and 2) aid in heat generation during cold weather where shaky and shivering would be detrimental (or dangerous) to the performance of a task.

In some cases, the disclosed device may be used for active therapeutic. For example, patients suffering from certain conditions may be prone to hypo/hyper-thermia. Therapeutic garments may be used as means to relieve symptoms or improve recovery.

Alignment of Heat Elimination and Activity Signals, Over Time and/or Over a Circadian Cycle, as an Independent Measure of Health

When utilizing thermal fit as the core health metric, health can be seen as a positive correlation between heat generation (circadian) rhythm and heat elimination (circadian) rhythm. Eliminating heat may be necessary to maintain parity of work/activity (heat generation) and heat elimination, thus maintain thermal regulation. Lack of health (unhealthy) can be shown by high degrees of variation, for example, in night-time heat elimination. In some embodiments, the types of disease/disorders that can be identified by the “thermal fit” tracking include infection, oncologic (cancer), metabolic disorders (for example, diabetes, metabolic syndrome), traumatic disease, or poisoning.

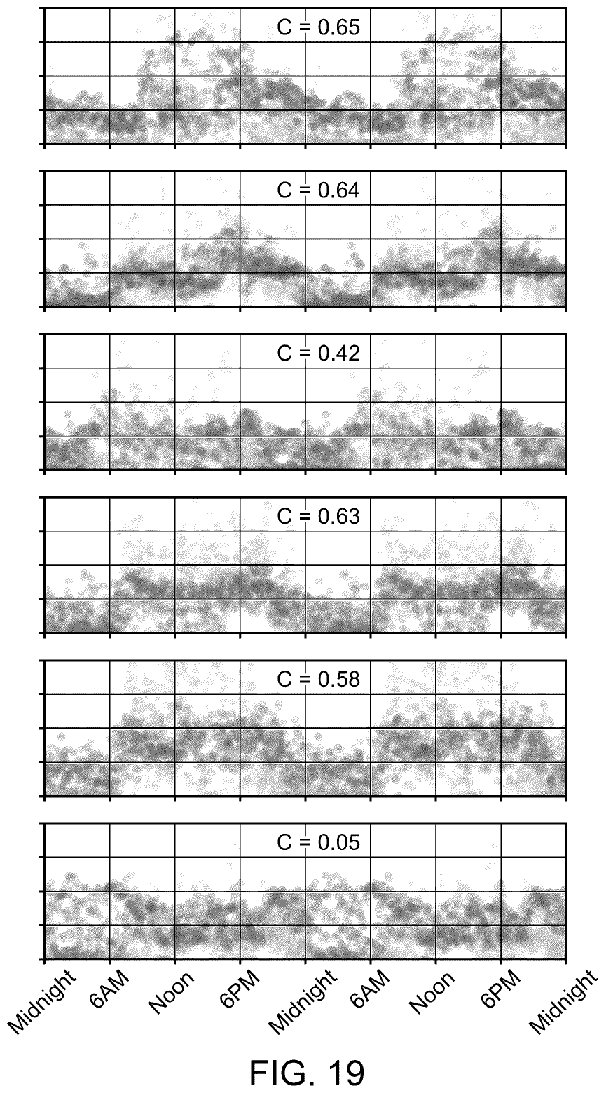

In one example of utilizing “thermal fit” as the core health metric, an experiment was performed by tracking heat generation and heat elimination on a circadian rhythm cycle or multiple circadian rhythm cycles. The heat generation was tracked via measuring activity. FIG. 19 shows measured data from such an experiment.

In particular, in the experiment, a measurement device was worn on the wrist or ankle of a person. The measurement device was paired by Bluetooth Low Energy to a mobile phone. The mobile phone was used to relay and aggregate data from device over several weeks. In some cases, the measurement device used applications which include automated relay of all data to a cloud storage from which it can be retrieved later. The measurement device includes accelerometers and temperature sensors. The measurement intervals were scheduled (for example, 30 second sampling rate) by device firmware and sensor measurements were recorded to device memory in a normalized form.

Specifically, two temperatures, skin temperature and air temperature, were measured with an error of approximately 0.1 degree Celsius. The quantity differential temperature was computed as dT=Tₛₖᵢₙ−Tₐᵢᵣ, and was interpreted as a proxy of heat elimination. Furthermore, an actigraphy signal measured acceleration at 30 Hz sampling rate. The measured acceleration was down-sampled to a single value, the highest absolute value acceleration, every 30 seconds.

Regarding analysis, the differential temperature and activity were again down-sampled as the mean at 5 minute resolution, i.e., all data points in each 5 minute period are averaged together. The activity signal was multiplied by 16 so that it will be roughly the same scale as the differential temperature. This rescaling does not impact the correlation of the two signals.

In FIG. 19, which is a non-limiting example of Thermal Fit data, all data points are plotted against time of day (this is just the date time of each data point with the date information stripped away). To make the pattern clearer visually, two cycles of the circadian pattern are depicted (see double plotted actogram). In FIG. 19, for example, the activity is shown in mint green, differential temperature is shown in grey.

The data in FIG. 19 were generated using the same protocol, as follows:

The data set was depicted twice and showed two days worth of the patterns. This manner of displaying data related to circadian rhythm is conventional; it shows more clearly how one day flows into the next. Including a new version below which reflects this point with a new line added in red;
Device worn on wrist or ankle;
Device includes accelerometer and temperature sensors;
Measurement intervals are scheduled (30 second sampling rate) by device firmware and sensor measurements are recorded to device memory in a normalized form;
Two temperatures (skin and air) with error ˜0.1 degree Celsius;
Differential temperature computing as dT=T_skin-T_air, and interpreted as a proxy of heat elimination;
An actigraphy signal measured acceleration at 30 Hz sampling, downsampled to a single value (highest absolute value acceleration) every 30 seconds;
Device paired by BLE to a mobile phone;
Mobile phone used to relay and aggregate data from device over weeks;
Initially not automated, data transferred and concatenated by hand;
More recent app versions include automated relay of all data to an ΔWS cloud storage from which it can be retrieved later;
To begin analysis the differential temperature and activity are again down-sampled as the mean at 5 minute resolution; all data points in each 5 minute period are averaged together;
The activity signal is multiplied by 16 so that it will be roughly the same scale as the differential temperature (this rescaling does not impact the correlation of the two signals);
All data points are plotted against time of day (this is just the date time of each data point with the date information stripped away);
To make the pattern clearer visually, two cycles of the circadian pattern are depicted as is typical in circadian rhythm community (see double plotted actogram);
The activity is shown as a mint green, differential temperature is in grey;
Correlation scores are computed as a Spearman R coefficient across the entire down-sampled dataset;
No removal of outliers;
No other data cleaning except for the previously mentioned downsampling operations which are merely meant to make the figures more readable by reducing the number of points;
Plot generated from five individuals, one individual appears twice (once each for ankle and wrist measurements), for a total of six panels;
The first five panels are derived from metabolically healthy individuals while the final panel shows a brittle diabetic patient which kidney failure

In FIG. 19, a plot was generated for each of five individuals, one individual appears twice (once each for ankle and wrist measurements), for a total of six panels. The first five panels are derived from metabolically healthy individuals while the final panel shows a brittle diabetic patient which kidney failure.

Further, the correlation scores in FIG. 19 were computed as a Spearman R coefficient across the entire down-sampled dataset. There was no removal of outliers and no other data cleaning, except for the aforementioned down-sampling operations which merely made the figures more readable by reducing the number of points.

By way of example, FIG. 19 illustrates an activity corrected heat signature, a way of subtracting the effect of activity from the heat signature. In some cases, significant alignment between heat and activity occurs often, and a peak in activity may generate heat and therefore a simultaneously peak in heat elimination. However, because the body can store heat, peaks in heat and activity may not be completely aligned. For example, activity may generate heat which is stored and released later. This can be healthy or unhealthy in various circumstances.

The degree of alignment between heat generation and heat elimination may be considered a primary feature of thermal fit. For example, FIG. 19 compares the differential temperature with an activity signal. Except for the last panel, the differential temperature and the activity signal are generally “aligned” or have high correlation. The last panel shows data from a diabetic subject, and the data exhibits some significant discordance between differential temperature and activity.

The last panel of FIG. 19 shows data having such striking discordance that there may be limited need for Artificial Intelligence (AI) learning for purposes of triage or diagnosis. Without being bound by theory, this can be explained by noting that diabetes causes defects in capillary systems, and the extensive, complex capillary network in the skin is responsible for regulating heat elimination through the skin. Even though AI might not be necessary, AI may prove to be helpful in tracking particular nuances of thermal fit patterns, and in correlating those patterns to particular disease/disorders.

In some embodiments, to be used as metrics which compare the temporal shape of two metrics where one is related to heat generation and one is related to heat elimination, Pearson and Spearman correlations between an accelerometer signature and differential temperature obtained from the measurement device can be computed. In other embodiments, A z-transform (or its special cases, the Fourier and Laplace transforms) of the activity signature is compared to z-transform of the heat elimination in such a manner that various aspects of frequency and phase response of the two signals can be compared. In further embodiments, an autoencoding convolutional neural network can be trained on a large corpus of data pertaining to heat generation and elimination to learn a low rank representation of these physiological time series. Convolutional kernels within the network can be identified which correspond to an orthogonal set of “alignment classes” which exhibit distinct temporal properties in terms of heat generation, storage and elimination, which play out in ways which cannot be expressed concisely as an infinite series (e.g. z-transform or other parametric integral transform). The activations of these “alignment kernels” are then computed in real time as scores which indicate membership in a finite set of “alignment classes” which may map on to clinical or laboratory-validated states of health/disease.

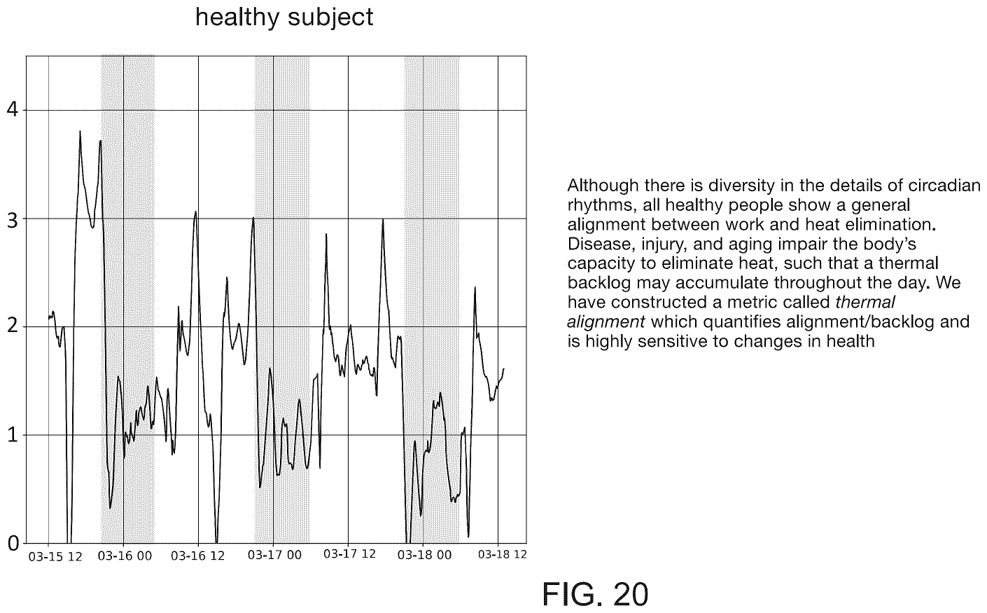

FIG. 20 illustrates that, although there is diversity in the details of circadian rhythms, all healthy people show a general alignment between work and heat elimination; it also illustrates that disease, injury, and aging impair the body's capacity to eliminate heat, such that a thermal backlog may accumulate throughout the day, and a metric called thermal alignment quantifies alignment/backlog and is highly sensitive to changes in health.

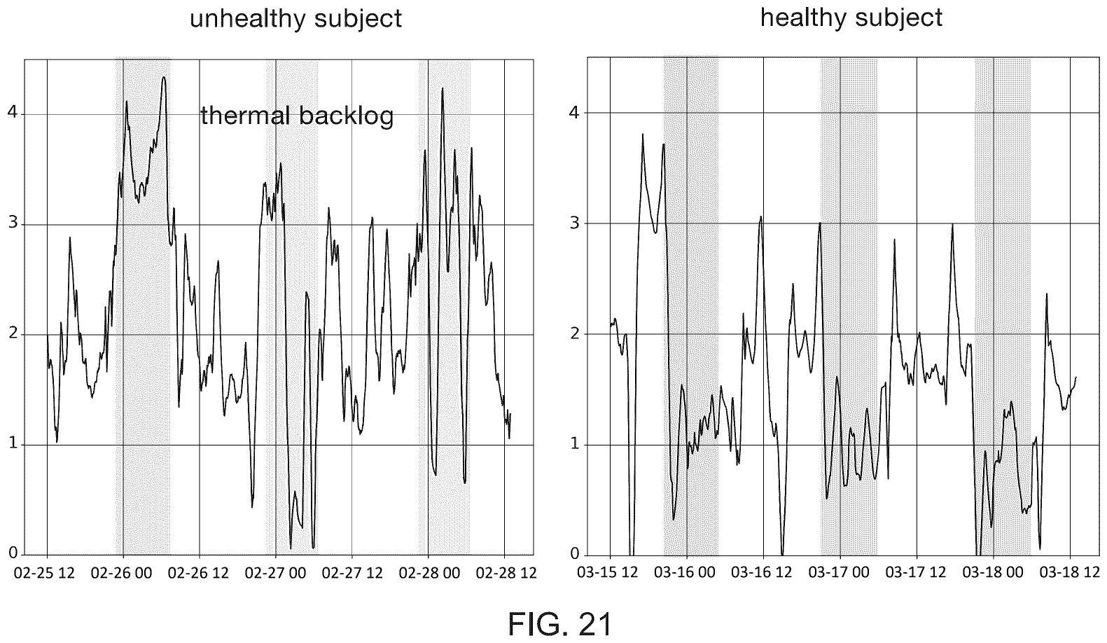

FIG. 21 illustrates thermal misalignment through thermal backlog in unhealthy subjects and compares this to thermal alignment in a healthy subject.

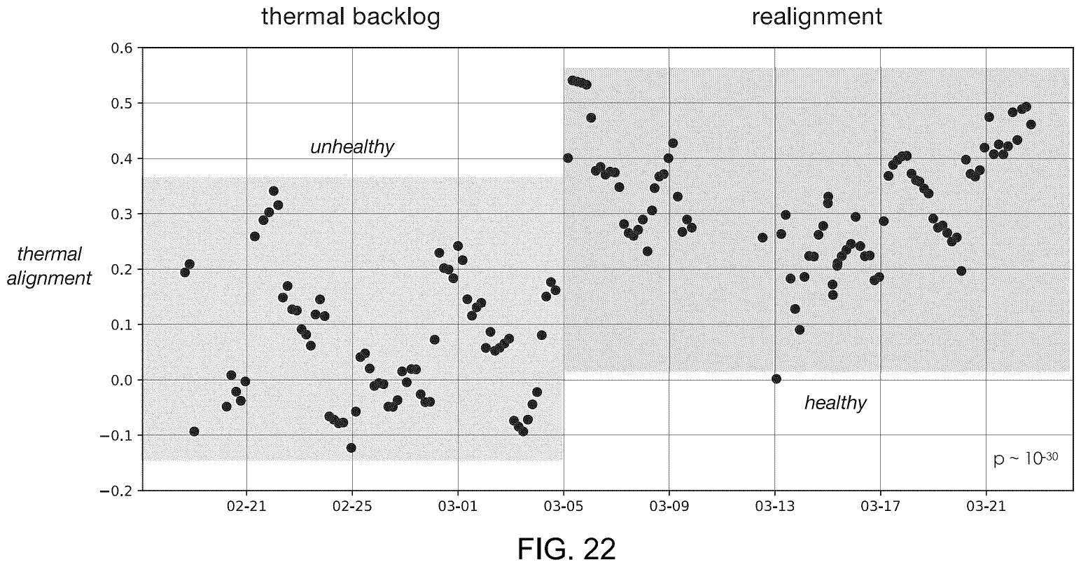

FIG. 22 illustrates the effect of treatment, showing thermal backlog in an unhealthy subject, and the effect of treatment to achieve thermal realignment.

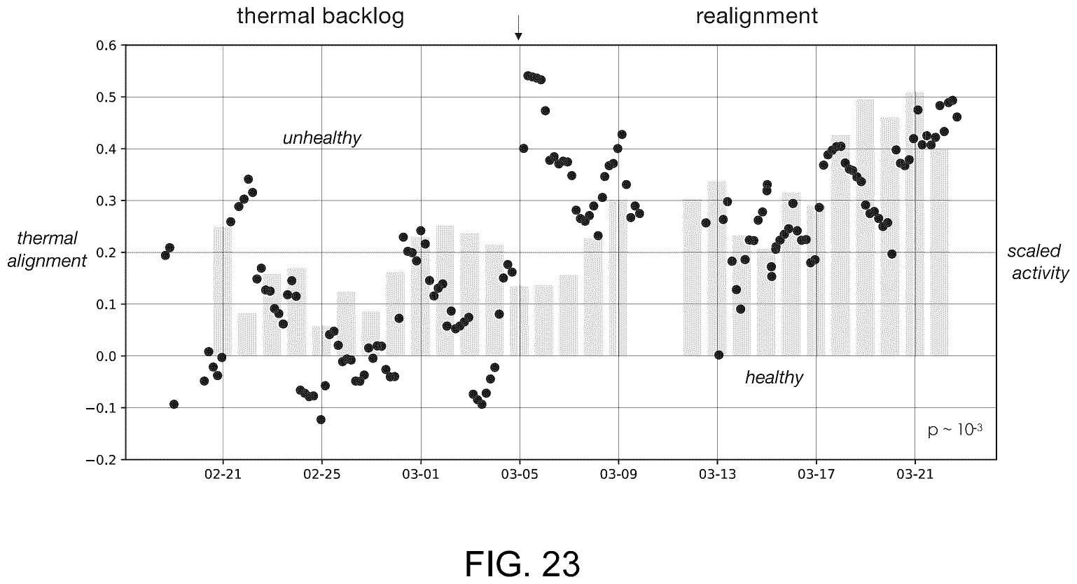

FIG. 23 illustrates a correlation of treatment to improved activity by reference to “thermal fit.”

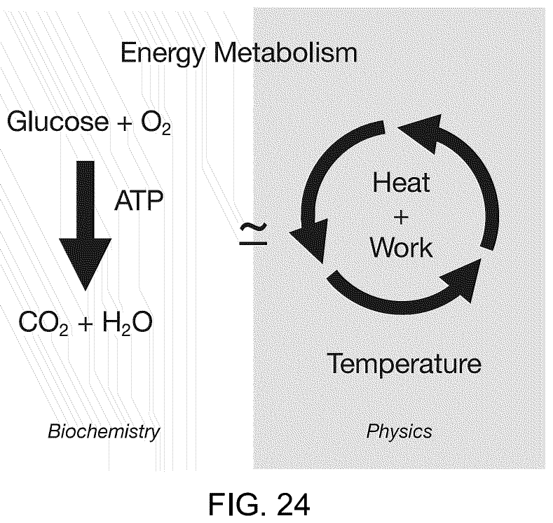

FIG. 24 illustrates that “thermal fit” is a scalable vital sign of energy metabolism. Physiologic work and heat elimination are quantitatively related to oxygen consumption. A wearable device may be used to continuously measure heat and work, therefore addresses and overcomes the limitations in scalability of VO2 stress testing/CRF. Moreover, a new metric of health, thermal fit, may be measured by the wearable device. Thermal fit is a measure of thermal alignment—the mathematical relationship between heat elimination and work that enables the stabilization of core temperature to maintain health.

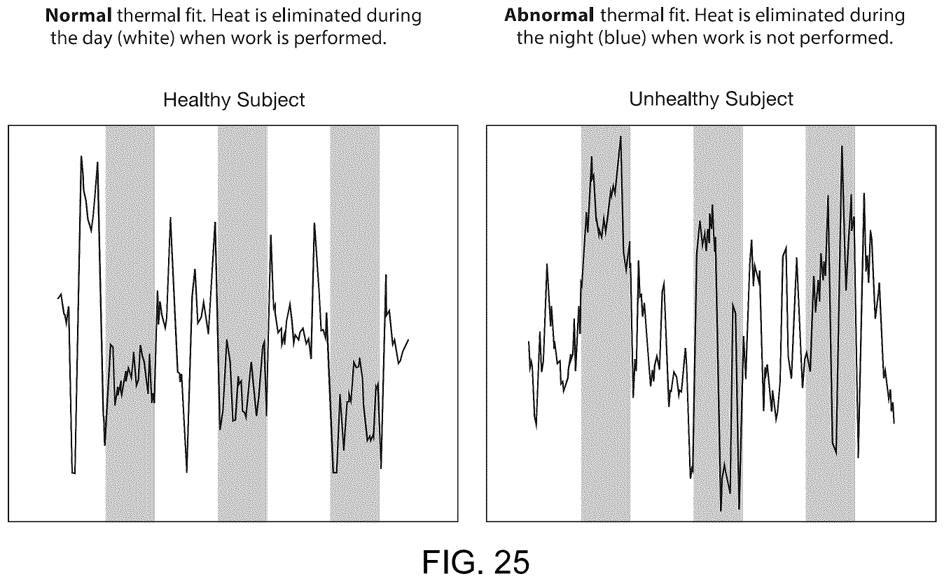

FIG. 25 illustrates “thermal fit” in a healthy subject versus in an unhealthy subject. In a healthy subject with normal thermal fit, heat is eliminated during the day (white) when work is performed. In an unhealthy subject with abnormal thermal fit, heat is eliminated during the night (grey) when work is not performed.

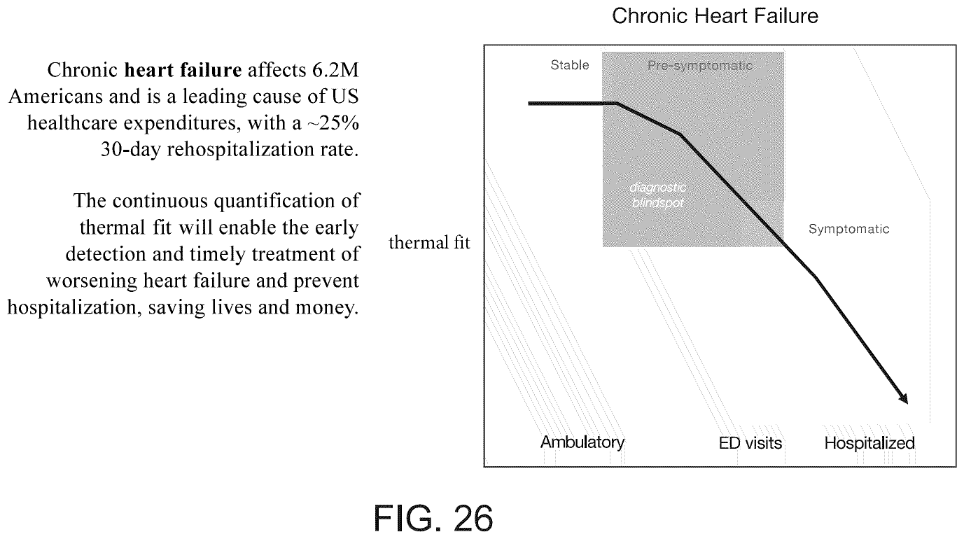

FIG. 26 illustrates how the “thermal fit” indicator can be used for pre-symptomatic diagnosis and timely treatment. Chronic heart failure affects 6.2M Americans and is a leading cause of US healthcare expenditures, with a ˜25% 30-day rehospitalization rate. The continuous quantification of thermal fit will enable the early detection and timely treatment of worsening heart failure and prevent hospitalization, saving lives and money.

### Case 1: Human

In some embodiments, the disclosed technology may be used to diagnose health in humans. In humans, factors that increase Health Capacity may be sleep, physical exercise, nutrition, lifestyle, or inputs to the brain through all senses (enrichment). The disclosed technology may make recommendations regarding adjusting the factors that increase Health Capacity to improve health, increase fitness, lessen frailty, lessen (prevent or intercept) disease, increase lifespan, increase fertility, increase physical performance, improve fetal/maternal health (pregnancy), improve brain/CNS performance, or change appearance.

In some embodiments, the disclosed technology may be used to diagnose disease in humans. In humans, Health Capacity may be decreased by factors such as infection, trauma, iatrogenic, cancer, metabolic abnormalities, genetic abnormalities, inactivity, disengagement, ageing, inflammation, malnutrition, over-nutrition, starvation, poisonings, de-enrichment (opposite of enrichment). The disclosed technology may make recommendations to increase Health Capacity in humans and to increase fitness, lessen frailty, lessen (prevent, intercept) disease, increase lifespan, increase fertility, increase physical performance, improve fetal/maternal health (pregnancy), improve brain/CNS performance, or change appearance.

### Case 2: Non-Human

In some embodiments, the disclosed technology may be used to diagnose health in companion animals. In companion animals, factors that increase Health Capacity may be sleep, physical exercise, nutrition, lifestyle, or inputs to the brain through all senses (enrichment). The disclosed technology may make recommendations regarding adjusting the factors that increase Health Capacity in companion animals to improve health, increase fitness, lessen frailty, lessen (prevent or intercept) disease, increase lifespan, increase fertility, increase physical performance, improve fetal/maternal health (pregnancy), improve brain/CNS performance, or change appearance.

In some embodiments, the disclosed technology may be used to diagnose disease in companion animals. In companion animals, Health Capacity may be decreased by factors such as infection, trauma, iatrogenic, cancer, metabolic abnormalities, genetic abnormalities, inactivity, disengagement, ageing, inflammation, malnutrition, over-nutrition, starvation, poisonings, de-enrichment (opposite of enrichment). The disclosed technology may make recommendations to increase Health Capacity in companion animals and to increase fitness, lessen frailty, lessen (prevent, intercept) disease, increase lifespan, increase fertility, increase physical performance, improve fetal/maternal health (pregnancy), improve brain/CNS performance, or change appearance.

In some embodiments, the disclosed technology may be used for manufacturing, spoilage, tracking farming animals.

In some embodiments, the disclosed technology may be used for manufacturing, spoilage, tracking farming plants, or changing the manufacturing quantity or quality of plants.

### Case 3: Industrial or Synthetic Biology

In some embodiments, the disclosed technology may be used for manufacturing chemicals, cells, organs, catalysts-bioremediation, or suspended animation.

### Case 4: Models of Humans, Non-Humans, Synthetic/Industrial

In some embodiments, the disclosed technology may be used for improved modeling of human health and human response to various stimuli including, for example, nutritional, environmental, of physical stresses. The disclosed technology may also be used for improved modeling of synthetic systems or of industrial systems, including such systems responses to various stresses including, for example, input limitations, outflow limitations, manufacturing demands, and energy consumption restrictions.

### Case 5: Others Implementations of the Disclosed Technology

In some embodiments, aspects of the disclosed technology may be applied to improve encryption, by using energy signature information or any energy signature-based representation. Aspects of the disclosed technology may be applied to improve ecology, especially in the context of carbon credit trading. Aspects of the disclosed technology may be applied to may be used in the context of the insurance industry especially with respect to implementing real time risks allocation and pricing. Aspects of the disclosed technology may be applied to may be used in the field of cybernetic, especially in the context if human/machine interfaces. Aspects of the disclosed technology may be applied to may be used in the field of art, especially with respect to expressions of energy signatures. Aspects of the disclosed technology may be applied to may be used in the gaming industry, especially as biomimetics may be used that are based on energy budget rules.

What is claimed is:

1. A system for quantifying resilience of a biological system comprising:
one or more sensors configured to measure data from the biological system indicative of an emergent factor of the biological system; and
one or more processors configured to process the measured data to derive a resilience metric of the biological system, the resilience metric representing energy and information contained in the biological system that enable persistence and/or function of the biological system.

2. The system of claim 1, wherein the one or more processors are further configured to compute a solution for maximizing the resilience metric of the biological system according to machine readable instructions.

3. The method of claim 2, wherein the solution comprises a recommendation to modify one or more stimuli that influences the resilience metric of the biological system, wherein the stimuli are selected from sleep patterns, sleep durations, nutritional intakes, and exercise regimens.

4. The system of claim 1, further comprising a plurality of transmitters configured to transmit the measured data as data streams optimized with respect to properties of the one or more sensors and the emergent factors to be reported.

5. The system of claim 1, wherein the one or more processors are further configured to make pre-symptomatic detection of a disease of the biological system based on the derived resilience metric.

6. The system of claim 1, wherein the one or more sensors comprise a plurality of wearable devices for measuring data comprising at least one of heat flux data, calorimetry data, osmometry data, and physiometry data when worn by the biological system.

7. The system of claim 1, wherein the measured data comprises surface temperature and/or physical activity of the biological system over time.

8. The system of claim 7, wherein the one or more processors are configured to process the measured data to derive a resilience metric of the biological system based on:
estimating heat elimination of the biological system over time based on differential surface temperature;
estimating heat production of the biological system over time based on physical activity;
estimating a metabolic status of the biological system based on temporal alignment of heat elimination and heat production; and
determining the resilience metric by applying a time-dependent function to the estimated metabolic status, wherein the time-dependent function is derived from a quasiperiodic rhythm of the biological system.

9. The method of claim 8, wherein the quasiperiodic rhythm is a circadian rhythm.

10. The method of claim 8, wherein the emergent factor is the temporal alignment of heat production and heat elimination.

11. The system of claim 8, wherein the one or more processors are further configured to generate and output at least one indicator for improving or modulating the temporal alignment of heat production and heat elimination of the biological system.

12. The system of claim 11, wherein the at least one indicator suggests to the biological system to perform to at least one predefined action selected from the group of actions comprises: change clothes, go inside, go outside, eat a specific food, drink a specified beverage, perform certain exercises, go to sleep, or any combination thereof.

13. The system of claim 1, wherein the one or more processors are further configured to analyze functional aspects of thermoregulation in the biological system based on the measured data.

14. The system of claim 1, wherein the one or more processors are further configured to generate and output indicators used to manage weight, blood pressure, circadian rhythm, sleep quality, sleep duration, or any combination thereof, of the biological system based on the derived resilience metric.

15. A method for quantifying resilience of a biological system comprising:
measuring data from the biological system indicative of an emergent factor of the biological system; and
processing the measured data to derive a resilience metric of the biological system, the resilience metric representing energy and information contained in the biological system that enable persistence and/or function of the biological system.

16. The method of claim 15, further comprising computing a solution for maximizing the resilience metric of the biological system according to machine readable instructions.

17. The method of claim 16, wherein the solution comprises a recommendation to modify one or more stimuli that influences the resilience metric of the biological system, wherein the stimuli are selected from sleep patterns, sleep durations, nutritional intakes, and exercise regimens.

18. The method of claim 15, further comprising making pre-symptomatic detection of a disease of the biological system based on the derived resilience metric.

19. The method of claim 15, wherein the measured data comprises surface temperature and/or physical activity of the biological system over time.

20. The method of claim 15, wherein the step of processing the measured data to derive a resilience metric of the biological system comprises:
estimating heat elimination of the biological system over time based on differential surface temperature;
estimating heat production of the biological system over time based on physical activity;
estimating a metabolic status of the biological system based on temporal alignment of heat elimination and heat production; and
determining the resilience metric by applying a time-dependent function to the estimated metabolic status, wherein the time-dependent function is derived from a quasiperiodic rhythm of the biological system.

21. The method of claim 20, wherein the quasiperiodic rhythm is a circadian rhythm.

22. The method of claim 20, wherein the emergent factor is the temporal alignment of heat production and heat elimination.

23. The method of claim 20, further comprising generating and outputting at least one indicator for improving or modulating the temporal alignment of heat production and heat elimination of the biological system.

24. The method of claim 23, wherein the at least one indicator suggests to the biological system to perform to at least one predefined action selected from the group of actions comprises: change clothes, go inside, go outside, eat a specific food, drink a specified beverage, perform certain exercises, go to sleep, or any combination thereof.

25. The method of claim 15, further comprising analyzing functional aspects of thermoregulation in the biological system based on the measured data.

26. The method of claim 15, further comprising generating and outputting indicators used to manage weight, blood pressure, circadian rhythm, sleep quality, sleep duration, or any combination thereof, of the biological system based on the derived resilience metric.
